SEQUENCE LISTING (158 sequences)

SEQ ID NO: 1 - protein, 9 aa, synthetic construct
GIDFSNHYY

SEQ ID NO: 2 - protein, 9 aa, synthetic construct
IFSGDSAST

SEQ ID NO: 3 - protein, 14 aa, synthetic construct
CARGMSTNDWASDL

SEQ ID NO: 4 - protein, 7 aa, synthetic construct
ESINSIY

SEQ ID NO: 5 - skipped

SEQ ID NO: 6 - protein, 13 aa, synthetic construct
CQQSYDWGDVENT

SEQ ID NO: 7 - protein, 122 aa, synthetic construct
EVQLLESGGGLVQPGGSLRLSCAASGIDFSNHYYMYWVRQAPGKGLEWIGSIFSGDSASTYYADSAKGRFTISRDNSKNTLYLQMNSLRAEDTAVYYCARGMSTNDWASDLWGQGTLVTVSS

SEQ ID NO: 8 - protein, 111 aa, synthetic construct
DIQMTQSPSSLSASVGDRVTITCQASESINSIYLAWYQQKPGKAPKLLIYRASTLASGVPSRFSGSGSGTDFTLTISSLQPEDFATYYCQQSYDWGDVENTFGGGTKVEIK

SEQ ID NO: 9 - protein, 8 aa, synthetic construct
GYTFTDYN

SEQ ID NO: 10 - protein, 8 aa, synthetic construct
IYPSNGGT

SEQ ID NO: 11 - protein, 17 aa, synthetic construct
CARVPYYDYLYYYAMDY

SEQ ID NO: 12 - protein, 5 aa, synthetic construct
SSVSF

SEQ ID NO: 13 - skipped

SEQ ID NO: 14 - protein, 12 aa, synthetic construct
CQQWSTNPYMYT

SEQ ID NO: 15 - protein, 121 aa, synthetic construct
EVQLQQSGPEVKPGASVKISCKASGYTFTDYNMDWVKQSHGKSEWIGYIYPSNGGTGYNQKFKSKATLTVDKSSNTAYMELHSLTSEDSAVYYCARVPYYDYLYYYAMDYWGQGTSVTVSS

SEQ ID NO: 16 - protein, 108 aa, synthetic construct
QIVLSQSPAILSASPGEKVTMACRASSSVSFMHWYQQKPGSSPQPWIYATSNLASGVPARFSGSGSGTSYSLTISRVEAEDAATYYCQQWSTNPYMYTFGGGTKLEIK

SEQ ID NO: 17 - protein, 8 aa, synthetic construct
GFTFSSFG

SEQ ID NO: 18 - protein, 8 aa, synthetic construct
ISSGSSTI

SEQ ID NO: 19 - protein, 11 aa, synthetic construct
ARSTGTGAMDY

SEQ ID NO: 20 - protein, 6 aa, synthetic construct
QDISNY

SEQ ID NO: 21 - skipped

SEQ ID NO: 22 - protein, 9 aa, synthetic construct
QQGNTLPWT

SEQ ID NO: 23 - protein, 118 aa, synthetic construct
EVQLVESGGGLVQPGGSRKLSCAASGFTFSSFGMHWVRQAPEKGLEWVAYISSGSSTIYYADTVKGRFSISRDNPKNTLFLQMTSLRSEDTAMFYCARSTGTGAMDYWGQGTSVTVSS

SEQ ID NO: 24 - protein, 107 aa, synthetic construct
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLELK

SEQ ID NO: 25 - protein, 8 aa, synthetic construct
GFSLTSYG

SEQ ID NO: 26 - protein, 7 aa, synthetic construct
IWADGST

SEQ ID NO: 27 - protein, 10 aa, synthetic construct
AKGHWDGFAY

SEQ ID NO: 28 - protein, 7 aa, synthetic construct
SSITSSY

SEQ ID NO: 29 - skipped

SEQ ID NO: 30 - protein, 9 aa, synthetic construct
HQWSSYPPT

SEQ ID NO: 31 - protein, 116 aa, synthetic construct
EVQVVESGPGLVAPSQSLSITCTVSGFSLTSYGVSWVRQPPGKGLEWLGVIWADGSTNYHSALLSRLSISKDNSKSQVFLKLNSLQTDDTATYYCAKGHWDGFAYWGQGTLVAVSA

SEQ ID NO: 32 - protein, 108 aa, synthetic construct
DIVLTQSPAIMSTSPGEKVTLTCSASSSITSSYLYWYQQKPGSSPKLWIYSTSNLASGVPARFSGSGSGTSYYLTISSMEAEDAASYFCHQWSSYPPTFGGGTKLELK

SEQ ID NO: 33 - protein, 8 aa, synthetic construct
GFDFSRYW

SEQ ID NO: 34 - protein, 8 aa, synthetic construct
INPDSSTI

SEQ ID NO: 35 - protein, 14 aa, synthetic construct
ARPYYRYDVGYFDV

SEQ ID NO: 36 - protein, 6 aa, synthetic construct
QSVSNA

SEQ ID NO: 37 - skipped

SEQ ID NO: 38 - protein, 9 aa, synthetic construct
QQDYSSPWT

SEQ ID NO: 39 - protein, 121 aa, synthetic construct
EVKLLESGGGLVQPGGSLKLSCAASGFDFSRYWMIWVRQAPEKGLEWIGEINPDSSTINYTPSLKDKFIISRDNAKNTLYLQMSKVRSEDTALYYCARPYYRYDVGYFDVWGAGTTVTVSS

SEQ ID NO: 40 - protein, 107 aa, synthetic construct
DIVMTQAPKVLLVSAGDRVTISCKASQSVSNAVVWYQQKPGQSPKLLIFYASNRYTGVPDRFTGSGYGTDFTFSISTVQAEDLAVYFCQQDYSSPWTFGGGTKLEIK

SEQ ID NO: 41 - protein, 8 aa, synthetic construct
GFDFSRYW

SEQ ID NO: 42 - protein, 8 aa, synthetic construct
INPDSSAI

SEQ ID NO: 43 - protein, 8 aa, synthetic construct
ATSMTMDY

SEQ ID NO: 44 - protein, 6 aa, synthetic construct
QDIKSY

SEQ ID NO: 45 - skipped

SEQ ID NO: 46 - protein, 9 aa, synthetic construct
LQHGESPYT

SEQ ID NO: 47 - protein, 115 aa, synthetic construct
EVKVIESGGGLVQPGGSLKLSCAASGFDFSRYWMSWVRQAPGKGLEWIGEINPDSSAINYTPSLKDKFIISRDNAKNTLYLQMSKVRSEDTALYYCATSMTMDYWGQGTSVTVSS

SEQ ID NO: 48 - protein, 106 aa, synthetic construct
DIVMTQSPSSMYASLGERVTITCKASQDIKSYLSWYQQKPWKSPKTLIYYATSLADGVPSRFSGSGSGQDYSLTISSLESDDTATYYCLQHGESPYTFGGGTKLEK

SEQ ID NO: 49 - protein, 8 aa, synthetic construct
GFTFSSYA

SEQ ID NO: 50 - protein, 8 aa, synthetic construct
ISSGGSYI

SEQ ID NO: 51 - protein, 15 aa, synthetic construct
ARQDWDGGDYYAMDY

SEQ ID NO: 52 - protein, 12 aa, synthetic construct
QSVLYSSNQKNF

SEQ ID NO: 53 - skipped

SEQ ID NO: 54 - protein, 8 aa, synthetic construct
HQYLSSLT

SEQ ID NO: 55 - protein, 122 aa, synthetic construct
EVHLVESGGGLVKPGGSLKLSCAASGFTFSSYAMSWVRQTPEKRLEWVATISSGGSYIYYPDSVKGRFTISRDNAKNTLYLQMSSLRSEDTAMYYCARQDWDGGDYYAMDYWGQGTSVTVSS

SEQ ID NO: 56 - protein, 112 aa, synthetic construct
DIVMTQSPSSLAVSAGEKVTMSCKSSQSVLYSSNQKNFLAWYQQKPGQSPKLLIYWASTRESGVPDRFTGSGSGTDFTLTISSVQAEDLAVYYCHQYLSSLTFGAGTKLELK

SEQ ID NO: 57 - protein, 8 aa, synthetic construct
GFTFSSFG

SEQ ID NO: 58 - protein, 8 aa, synthetic construct
ISSGSSTI

SEQ ID NO: 59 - protein, 11 aa, synthetic construct
ARSTGTGAMDY

SEQ ID NO: 60 - protein, 6 aa, synthetic construct
QDISNY

SEQ ID NO: 61 - skipped

SEQ ID NO: 62 - protein, 9 aa, synthetic construct
QQGNALPWT

SEQ ID NO: 63 - protein, 118 aa, synthetic construct
EVQLVESGGGLVQPGGSRKLSCAASGFTFSSFGMHWVRQAPEKGLEWVAYISSGSSTIYYADTVKGRFTISRDNPKNTLFLQMTSLRSEDTAMYYCARSTGTGAMDYWGQGTSVTVSS

SEQ ID NO: 64 - protein, 107 aa, synthetic construct
DIQMTQTTSSLSASLGDSVTFSCRASQDISNYLNWYQQKPDGTVKLLISYTSRLHSGVPSRFSGSGSGTDYSLTISNLEQEDIATYFCQQGNALPWTFGGGTKLEIK

SEQ ID NO: 65 - protein, 8 aa, synthetic construct
GFTFSSYA

SEQ ID NO: 66 - protein, 7 aa, synthetic construct
ITIGDDT

SEQ ID NO: 67 - protein, 16 aa, synthetic construct
ARGVNSYGSSYRYFDV

SEQ ID NO: 68 - protein, 6 aa, synthetic construct
QDVSTA

SEQ ID NO: 69 - skipped

SEQ ID NO: 70 - protein, 9 aa, synthetic construct
QQHYSIPFT

SEQ ID NO: 71 - protein, 122 aa, synthetic construct
EVQLVESGGGLVKPGGSLKLSCAASGFTFSSYAMSWVRQTPEKRLEWVASITIGDDTYYPDSVKGRFTISRDHAKNILYLQVSSLRSEDTAMYSCARGVNSYGSSYRYFDVWGAGTTVTVSS

SEQ ID NO: 72 - protein, 106 aa, synthetic construct
DIVMTQSHKFMSTSVGDRVSITCKASQDVSTAVAWYQQKPGQSPKLLIYWASTRHTGVPDRFTGSGSGTDYTLTISSVQAEDLALYYCQQHYSIPFTFGAGTKLEK

SEQ ID NO: 73 - protein, 8 aa, synthetic construct
GFTFSSHG

SEQ ID NO: 74 - protein, 8 aa, synthetic construct
ISSGGIYT

SEQ ID NO: 75 - protein, 12 aa, synthetic construct
TRGQVGNWYFDV

SEQ ID NO: 76 - protein, 5 aa, synthetic construct
SSVSY

SEQ ID NO: 77 - skipped

SEQ ID NO: 78 - protein, 9 aa, synthetic construct
QQRSSYPLT

SEQ ID NO: 79 - protein, 119 aa, synthetic construct
EVHLVESGGDLVKPGGSLKLSCAASGFTFSSHGMSWVRQTPDKRLEWVATISSGGIYTYHPDSVKGRFSISRDNAKKTLYLQMSSLKSEDTGIYYCTRGQVGNWYFDVWGAGTTVTVSS

SEQ ID NO: 80 - protein, 106 aa, synthetic construct
DIVITQSPAIMSASPGEKVTINCSASSSVSYMHWFQQTPGTSPKLWIYSTSNLASGVPARFSGSGSGTFYSLTISRMEAEDAATYYCQQRSSYPLTFGAGTKLELK

SEQ ID NO: 81 - protein, 8 aa, synthetic construct
GFTFSSFG

SEQ ID NO: 82 - protein, 8 aa, synthetic construct
ISSGSSAM

SEQ ID NO: 83 - protein, 11 aa, synthetic construct
ARSDGKGAMDY

SEQ ID NO: 84 - protein, 6 aa, synthetic construct
QDISNY

SEQ ID NO: 85 - skipped

SEQ ID NO: 86 - protein, 9 aa, synthetic construct
QQGNTLPWT

SEQ ID NO: 87 - protein, 118 aa, synthetic construct
EVQRVESGGGLVQPGGSRKLSCAASGFTFSSFGMHWVRQAPEKGLEWVAYISSGSSAMYYADTVKGRFTISRDNPKNTLFLQMTSLRSEDTAMYYCARSDGKGAMDYWGQGTSVTVSS

SEQ ID NO: 88 - protein, 107 aa, synthetic construct
DIQMTQTTSSLSASLGDRVTISCRASQDISNYLNWYQQKPDGTVKLLVYYTSRLHSGVPSRFSGSGSGTDYSLTISNLEQEDIATYFCQQGNTLPWTFGGGTKLEIK

SEQ ID NO: 89 - protein, 8 aa, synthetic construct
GYTFTNYW

SEQ ID NO: 90 - protein, 8 aa, synthetic construct
INPSNGRT

SEQ ID NO: 91 - protein, 14 aa, synthetic construct
ARSTYYGYDWYFDV

SEQ ID NO: 92 - protein, 6 aa, synthetic construct
QDINSY

SEQ ID NO: 93 - skipped

SEQ ID NO: 94 - protein, 9 aa, synthetic construct
LQCDEFPHT

SEQ ID NO: 95 - protein, 121 aa, synthetic construct
EVQLQQSGTELVKPGASVKLSCKASGYTFTNYWMHWVKQRPGQGLEWIGEINPSNGRTNYNEKFKTKATLTVDKSSSTAYMQLSSLTSEDSAVYYCARSTYYGYDWYFDVWGAGTTVTVSS

SEQ ID NO: 96 - protein, 107 aa, synthetic construct
DIVLTQSPSSMYASLGERVTITCKASQDINSYLSWFQQKPGKSPKTLIYRANRLLDGVPSRFSGGGSGQDYSLTISSLEHEDMGIYYCLQCDEFPHTFGGGTKLEIK

SEQ ID NO: 97 - protein, 8 aa, synthetic construct
GFTFSTFG

SEQ ID NO: 98 - protein, 8 aa, synthetic construct
ISSGSSTI

SEQ ID NO: 99 - protein, 11 aa, synthetic construct
ARSTGTGAMDY

SEQ ID NO: 100 - protein, 6 aa, synthetic construct
QDISKY

SEQ ID NO: 101 - skipped

SEQ ID NO: 102 - protein, 9 aa, synthetic construct
QQGNTLPWT

SEQ ID NO: 103 - protein, 118 aa, synthetic construct
EVQLVESGGGLVQPGGSRKLSCAASGFTFSTFGMHWVRQAPEKGLEWVAYISSGSSTIYYADTVKGRFTISRDNPKNTLFLQMTSLRSEDTAMYYCARSTGTGAMDYWGQGTSVTVSS

SEQ ID NO: 104 - protein, 107 aa, synthetic construct
DIQMTQTTSSLSASLGDRVTISCRASQDISKYLNWYQQKPDGTVKLLIYYTSRLHSGVPSRFSGSGSGTDYSLTISNLEQEDIATYFCQQGNTLPWTFGGGTKLEIK

SEQ ID NO: 105 - protein, 8 aa, synthetic construct
GFTFSSHG

SEQ ID NO: 106 - protein, 8 aa, synthetic construct
INSGGYYT

SEQ ID NO: 107 - protein, 12 aa, synthetic construct
ARGQLGNWYFDV

SEQ ID NO: 108 - protein, 5 aa, synthetic construct
SSVSY

SEQ ID NO: 109 - skipped

SEQ ID NO: 110 - protein, 9 aa, synthetic construct
QQRSSYPLT

SEQ ID NO: 111 - protein, 119 aa, synthetic construct
EVQLVESGGDLVKPGGSLKLSCAASGFTFSSHGMSWVRQTPDKRLEWVATINSGGYYTYYPDSVKGRFTISRDNAKNTLYLQMSSLKSEDTAMYYCARGQLGNWYFDVWGAGTTVTVSS

SEQ ID NO: 112 - protein, 106 aa, synthetic construct
DIVMTQSPAIMSASPGEKVTITCSASSSVSYMHWFQQKPGTSPKLWIYSTSNLASGVPARFSGSGSGTSYSLTISRMEAEDAATYYCQQRSSYPLTFGAGTKLELK

SEQ ID NO: 113 - protein, 8 aa, synthetic construct
GFTFSSFG

SEQ ID NO: 114 - protein, 8 aa, synthetic construct
ISSGSSSI

SEQ ID NO: 115 - protein, 11 aa, synthetic construct
ARGTGTGAMDY

SEQ ID NO: 116 - protein, 6 aa, synthetic construct
QDISNY

SEQ ID NO: 117 - skipped

SEQ ID NO: 118 - protein, 9 aa, synthetic construct
QQGNTLPWT

SEQ ID NO: 119 - protein, 118 aa, synthetic construct
EVHLVESGGGLVQPGGSRKLSCAASGFTFSSFGMHWVRQPPEKGLEWVAYISSGSSSIYYADTVKGRFTISRDNPKNTLFLQMTSLRSEDTAMYYCARGTGTGAMDYWGQGTSVTVSS

SEQ ID NO: 120 - protein, 107 aa, synthetic construct
DIQMTQTTSSLSASLGDRVTISCRTSQDISNYLNWYQQKPDGTVKLLIYYTSKLHSGVPSRFSGSGSGTDYSLTISNLDQEDIATYFCQQGNTLPWTFGGGTKLEIK

SEQ ID NO: 121 - protein, 8 aa, synthetic construct
GFTFSVFG

SEQ ID NO: 122 - protein, 8 aa, synthetic construct
ISSGSNTI

SEQ ID NO: 123 - protein, 11 aa, synthetic construct
ARSSGTGAMDY

SEQ ID NO: 124 - protein, 6 aa, synthetic construct
QDISNY

SEQ ID NO: 125 - skipped

SEQ ID NO: 126 - protein, 9 aa, synthetic construct
QQGNTLPWT

SEQ ID NO: 127 - protein, 118 aa, synthetic construct
EVQLVESGGGLVQPGGSRKLSCAASGFTFSVFGVHWVRQAPEKGLEWVAYISSGSNTIHYADTVKGRFTISRDIPKNTLFLQMTSLRSEDTAMYYCARSSGTGAMDYWGQGTSVTVSS

SEQ ID NO: 128 - protein, 107 aa, synthetic construct
DIQMTQSTSSLSASLGDRVSISCRASQDISNYLNWYQQKPDGAVKLLIYYTSRLHSGVPSRFSGSGSGTDYSLTISNLDQEDIATYFCQQGNTLPWTFGGGTKLELK

SEQ ID NO: 129 - protein, 8 aa, synthetic construct
GFTFSSFG

SEQ ID NO: 130 - protein, 8 aa, synthetic construct
ISSGSSTI

SEQ ID NO: 131 - protein, 11 aa, synthetic construct
ARSDGKGAMDY

SEQ ID NO: 132 - protein, 6 aa, synthetic construct
QDISNY

SEQ ID NO: 133 - skipped

SEQ ID NO: 134 - protein, 9 aa, synthetic construct
QQGNTLPWT

SEQ ID NO: 135 - protein, 118 aa, synthetic construct
EVQRVESGGGLVQPGGSRKLSCAASGFTFSSFGMHWVRQAPEKGLEWVAYISSGSSTIYYADTVKGRFTISRDNPKNTLFLQMTSLRSEDTAMYYCARSDGKGAMDYWGQGTSVTVSS

SEQ ID NO: 136 - protein, 107 aa, synthetic construct
DIVMTQSTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLEIK

SEQ ID NO: 137 - protein, 8 aa, synthetic construct
GYTFTSHW

SEQ ID NO: 138 - protein, 8 aa, synthetic construct
INPSNGRT

SEQ ID NO: 139 - protein, 13 aa, synthetic construct
ARGNLTTVVPFDY

SEQ ID NO: 140 - protein, 6 aa, synthetic construct
QDINKF

SEQ ID NO: 141 - skipped

SEQ ID NO: 142 - protein, 8 aa, synthetic construct
LQYDLLLT

SEQ ID NO: 143 - protein, 120 aa, synthetic construct
EVQLKESGAELVKPGASVKLSCKASGYTFTSHWMHWVKQRPGQGLEWIGEINPSNGRTNYNEKFTSKATLTVDQSSSTAYMQLSSLTSEDSAVYYCARGNLTTVVPFDYWGQGTALTVSS

SEQ ID NO: 144 - protein, 106 aa, synthetic construct
DIVMTQSPSSLSASLGGKVTITCKASQDINKFIAWYQHKPGKGPRLLIHFTSTLQPGIPSRFSGSGSGRDYSFSISNLEPEDIATYFCLQYDLLLTFGGGTKLEIK

SEQ ID NO: 145 - protein, 8 aa, synthetic construct
GYTFTNYW

SEQ ID NO: 146 - protein, 8 aa, synthetic construct
INPSNGRT

SEQ ID NO: 147 - protein, 13 aa, synthetic construct
ARGSLTTVVPFDY

SEQ ID NO: 148 - protein, 6 aa, synthetic construct
QDINNY

SEQ ID NO: 149 - skipped

SEQ ID NO: 150 - protein, 8 aa, synthetic construct
LQYDILLT

SEQ ID NO: 151 - protein, 120 aa, synthetic construct
EVQLQQSGAELVKPGASVKLSCKASGYTFTNYWMHWVKQRPGQGLEWIGEINPSNGRTNYNEKFKSKATLTVDKSSSTAYMQLSSLTSEDSAVYYCARGSLTTVVPFDYWGQGTTLTVSS

SEQ ID NO: 152 - protein, 105 aa, synthetic construct
DIQMTQTPSSLSASLGGKVTITCKASQDINNYIAWYQHKPGKGPRLLIHYTSTLQPGIPSRFSGSGSGRDYSFSISNLEPEDIATYFCLQYDILLTFGGGTKLEK

SEQ ID NO: 153 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 154 - protein, 118 aa, synthetic construct
EVQLVESGGGLVQPGGSLRLSCAASGFTFSSFGMHWVRQAPGKGLEWVAYISSGSSSIYYADTVKGRFTISRDNAKNSLYLQMNSLRAEDTAVYYCARGTGTGAMDYWGQGTLVTVSS

SEQ ID NO: 155 - protein, 107 aa, synthetic construct
DIQMTQSPSSLSASVGDRVTITCRTSQDISNYLNWYQQKPGKAPKLLIYYTSKLHSGVPSRFSGSGSGTDFTFTISSLQPEDIATYFCQQGNTLPWTFGGGTKLEIK

SEQ ID NO: 156 - protein, 115 aa, synthetic construct
EVQLVESGGGLVQPGGSLRLSCAASGFDFSRYWMSWVRQAPGKGLVWIGEINPESSAINYAPSLKDRFTISRDNAKNTLYLQMNSLRAEDTAVYYCATSMTMDYWGQGTLVTVSS

SEQ ID NO: 157 - protein, 107 aa, synthetic construct
DIQMTQSPSSLSASVGDRVTITCKASQDIKSYLSWYQQKPGKAPKTLIYYATSLAGGVPSRFSGSGSGTDYTLTISSLQPEDFATYYCLQHGESPYTFGGGTKLEIK

SEQ ID NO: 158 - protein, 8 aa, synthetic construct
INPESSAI

(19) United States
(12) Patent Application Publication
(10) Pub. No.: US 2025/0361295 A1
(43) Pub. Date: Nov. 27, 2025

(54) NOVEL ANTI-LILRB4 ANTIBODIES AND USES THEREOF

(71) Applicant: ANTENGENE BIOLOGICS LIMITED, Shanghai (CN)

(72) Inventors: Ao SUN, Shanghai (CN); Enlin ZHENG, Shanghai (CN); Mengshi GAO, Shanghai (CN); Suya BAI, Shanghai (CN); Peng CHEN, Shanghai (CN); Hui YUWEN, Shanghai (CN); Bing HOU, Shanghai (CN); Bo SHAN, Shanghai (CN); Jay MEI, Shanghai (CN)

(21) Appl. No.: 18/872,279

(22) PCT Filed: Jun. 5, 2023
(86) PCT No.: PCT/CN2023/098246

§ 371 Date: Dec. 6, 2024

(30) Foreign Application Priority Data
Jun. 6, 2022 (WO) PCT/CN2022/097121
May 24, 2023 (WO) PCT/CN2023/096031

(51) Int. Cl. C07K 16/28; A61K 39/00; A61P 35/00; G01N 33/574

(52) U.S. Cl. CPC C07K 16/2803; A61P 35/00; G01N 33/57484; A61K 2039/505; C07K 2317/24; C07K 2317/732; C07K 2317/76; C07K 2317/92; G01N 2333/70503

(57) ABSTRACT

The present disclosure provides anti-LILRB4 antibodies or antigen-binding fragments thereof, pharmaceutical composition comprising the same and the uses thereof.

### FIELD OF THE INVENTION

The present disclosure generally relates to novel anti-LILRB4 antibodies and antigen-binding fragments thereof and uses of the same.

### BACKGROUND

Acute myeloid leukemia (AML) is one of the most common acute leukemia of adults. To effectively treat AML, new molecular targets and therapeutic approaches shall be identified. The leukocyte immunoglobulin-like receptors (LILRs) are a family of at least 13 receptors mainly expressed by lymphoid and myelomonocytic cells (Rachel Thomas et al., Clin Rev Allergy Immunol., 2010 April; 38 (2-3): 159-62). In general, the LILR family includes LILRA1, LILRA2, LILRA3, LILRA4, LILRA5, LILRA6, LILRB1, LILRB2, LILRB3, LILRB4, LILRB5, LILRB6, and LILRB7. It is known that LILRBs are expressed on myeloid cells and certain other hematopoietic cells (Mori et al., J Immunol., 2008 Oct. 1; 181 (7): 4742-51). Several members of the LILRB family are highly expressed on AML cells, and their expression negatively correlates with the overall survival of human AML patients. In addition, inhibition of the expression of several LILRBs individually inhibited different human leukemia cell lines in culture and blocked leukemia development in xenografted mice (see, WO2013181438A2).

Leukocyte immunoglobulin-like receptor subfamily B member 4 (LILRB4) is a protein that in humans is encoded by the LILRB4 gene. The encoded protein belongs to the subfamily B class of LILRs, which contains two or four extracellular immunoglobulin domains, a transmembrane domain, and two to four cytoplasmic immunoreceptor tyrosine-based inhibitory motifs (ITIMs). The LILRB4 is expressed on monocytic cells and transduces a negative signal that inhibits stimulation of an immune response. The LILRB4 can also function in antigen capture and presentation. It is thought to control inflammatory responses and cytotoxicity to help focus the immune response and limit autoreactivity. LILRB4 has also been proposed to be a potential target for tumor immunotherapy. It has been shown to express on tumor-associated macrophages and negatively regulate immune response in tumor. The expression of LILRB4 on monocytic myeloid leukemia cells supports infiltration and inhibits T cell proliferation. IO-202, developed by Immune-Onc Therapeutics, is undergoing phase I clinical trial for treating AML and chronic myelomonocytic leukemia (CMML).

Needs remain for novel anti-LILRB4 antibodies.

### SUMMARY OF THE INVENTION

Throughout the present disclosure, the articles “a”, “an”, and “the” are used herein to refer to one or to more than one (i.e., to at least one) of the grammatical object of the article. By way of example, “an antibody” means one antibody or more than one antibody.

In one respect, the present disclosure provides an antibody or antigen-binding fragment thereof which binds to LILRB4, comprising:
one or two or three heavy chain complementarity determining regions (HCDR1, HCDR2 and/or HCDR3) contained within any one of the heavy chain variable (VH) region sequences selected from the group consisting of SEQ ID NOs: 23, 31, 39, 47, 55, 63, 71, 79, 87, 95, 103, 111, 119, 127, 135, 143, 151, 154 and 156; and/or
one or two or three light chain complementarity determining regions (LCDR1, LCDR2 and LCDR3) contained within any one of the light chain variable (VL) region sequences selected from the group consisting of SEQ ID NOs: 24, 32, 40, 48, 56, 64, 72, 80, 88, 96, 104, 112, 120, 128, 136, 144, 152, 155 and 157.

In some embodiments, the antibody or antigen-binding fragment thereof provided herein comprises at least one heavy or light chain complementarity determining region (CDR) comprising an amino acid sequence selected from the group consisting of SEQ ID NOs: 17, 18, 19, 20, 21, 22, 25, 26, 27, 28, 29, 30, 33, 34, 35, 36, 37, 38, 41, 42, 43, 44, 45, 46, 49, 50, 51, 52, 53, 54, 57, 58, 59, 60, 61, 62, 65, 66, 67, 68, 69, 70, 73, 74, 75, 76, 77, 78, 81, 82, 83, 84, 85, 86, 89, 90, 91, 92, 93, 94, 97, 98, 99, 100, 101, 102, 105, 106, 107, 108, 109, 110, 113, 114, 115, 116, 117, 118, 121, 122, 123, 124, 125, 126, 129, 130, 131, 132, 133, 134, 137, 138, 139, 140, 141, 142, 145, 146, 147, 148, 149, 150 and 158.

In some embodiments, the antibody or antigen-binding fragment thereof provided herein comprises one or two or three of HCDR1, HCDR2 and HCDR3 comprising an amino acid sequence selected from the group consisting of SEQ ID NOs: 17, 18, 19, 25, 26, 27, 33, 34, 35, 41, 42, 43, 49, 50, 51, 57, 58, 59, 65, 66, 67, 73, 74, 75, 81, 82, 83, 89, 90, 91, 97, 98, 99, 105, 106, 107, 113, 114, 115, 121, 122, 123, 129, 130, 131, 137, 138, 139, 145, 146, 147 and 158.

In some embodiments, the antibody or antigen-binding fragment thereof provided herein comprises one or two or three of LCDR1, LCDR2 and LCDR3 comprising an amino acid sequence selected from the group consisting of SEQ ID NOs: 20, 21, 22, 28, 29, 30, 36, 37, 38, 44, 45, 46, 52, 53, 54, 60, 61, 62, 68, 69, 70, 76, 77, 78, 84, 85, 86, 92, 93, 94, 100, 101, 102, 108, 109, 110, 116, 117, 118, 124, 125, 126, 132, 133, 134, 140, 141, 142, 148, 149 and 150.

In some embodiments, the antibody or antigen-binding fragment thereof provided herein comprises:
i. a HCDR1 comprising an amino acid sequence selected from the group consisting of SEQ ID NOs: 17, 25, 33, 41, 49, 57, 65, 73, 81, 89, 97, 105, 113, 121, 129, 137 and 145;
ii. a HCDR2 comprising an amino acid sequence selected from the group consisting of SEQ ID NOs: 18, 26, 34, 42, 50, 58, 66, 74, 82, 90, 98, 106, 114, 122, 130, 138, 146 and 158; and
iii. a HCDR3 comprising an amino acid sequence selected from the group consisting of SEQ ID NOs: 19, 27, 35, 43, 51, 59, 67, 75, 83, 91, 99, 107, 115, 123, 131, 139 and 147.

In some embodiments, the antibody or antigen-binding fragment thereof provided herein comprises:
i. a LCDR1 comprising an amino acid sequence selected from the group consisting of SEQ ID NOs: 20, 28, 36, 44, 52, 60, 68, 76, 84, 92, 100, 108, 116, 124, 132, 140 and 148;
ii. a LCDR2 comprising an amino acid sequence selected from the group consisting of SEQ ID NOs: 21, 29, 37, 45, 53, 61, 69, 77, 85, 93, 101, 109, 117, 125, 133, 141 and 149; and
iii. a LCDR3 comprising an amino acid sequence selected from the group consisting of SEQ ID NOs: 22, 30, 38, 46, 54, 62, 70, 78, 86, 94, 102, 110, 118, 126, 134, 142 and 150.

In some embodiments, the antibody or antigen-binding fragment thereof provided herein comprises:
i. a HCDR1 comprising an amino acid sequence as set forth in SEQ ID NO: 17, a HCDR2 comprising an amino acid sequence as set forth in SEQ ID NO: 18, and a HCDR3 comprising an amino acid sequence as set forth in SEQ ID NO: 19;
ii. a HCDR1 comprising an amino acid sequence as set forth in SEQ ID NO: 25, a HCDR2 comprising an amino acid sequence as set forth in SEQ ID NO: 26, and a HCDR3 comprising an amino acid sequence as set forth in SEQ ID NO: 27;
iii. a HCDR1 comprising an amino acid sequence as set forth in SEQ ID NO: 33, a HCDR2 comprising an amino acid sequence as set forth in SEQ ID NO: 34, and a HCDR3 comprising an amino acid sequence as set forth in SEQ ID NO: 35;
iv. a HCDR1 comprising an amino acid sequence as set forth in SEQ ID NO: 41, a HCDR2 comprising an amino acid sequence as set forth in SEQ ID NO: 42, and a HCDR3 comprising an amino acid sequence as set forth in SEQ ID NO: 43;
v. a HCDR1 comprising an amino acid sequence as set forth in SEQ ID NO: 41, a HCDR2 comprising an amino acid sequence as set forth in SEQ ID NO: 158, and a HCDR3 comprising an amino acid sequence as set forth in SEQ ID NO: 43;
vi. a HCDR1 comprising an amino acid sequence as set forth in SEQ ID NO: 49, a HCDR2 comprising an amino acid sequence as set forth in SEQ ID NO: 50, and a HCDR3 comprising an amino acid sequence as set forth in SEQ ID NO: 51;
vii. a HCDR1 comprising an amino acid sequence as set forth in SEQ ID NO: 57, a HCDR2 comprising an amino acid sequence as set forth in SEQ ID NO: 58, and a HCDR3 comprising an amino acid sequence as set forth in SEQ ID NO: 59;
viii. a HCDR1 comprising an amino acid sequence as set forth in SEQ ID NO: 65, a HCDR2 comprising an amino acid sequence as set forth in SEQ ID NO: 66, and a HCDR3 comprising an amino acid sequence as set forth in SEQ ID NO: 67;
ix. a HCDR1 comprising an amino acid sequence as set forth in SEQ ID NO: 73, a HCDR2 comprising an amino acid sequence as set forth in SEQ ID NO: 74, and a HCDR3 comprising an amino acid sequence as set forth in SEQ ID NO: 75;
x. a HCDR1 comprising an amino acid sequence as set forth in SEQ ID NO: 81, a HCDR2 comprising an amino acid sequence as set forth in SEQ ID NO: 82, and a HCDR3 comprising an amino acid sequence as set forth in SEQ ID NO: 83;
xi. a HCDR1 comprising an amino acid sequence as set forth in SEQ ID NO: 89, a HCDR2 comprising an amino acid sequence as set forth in SEQ ID NO: 90, and a HCDR3 comprising an amino acid sequence as set forth in SEQ ID NO: 91;
xii. a HCDR1 comprising an amino acid sequence as set forth in SEQ ID NO: 97, a HCDR2 comprising an amino acid sequence as set forth in SEQ ID NO: 98, and a HCDR3 comprising an amino acid sequence as set forth in SEQ ID NO: 99;
xiii. a HCDR1 comprising an amino acid sequence as set forth in SEQ ID NO: 105, a HCDR2 comprising an amino acid sequence as set forth in SEQ ID NO: 106, and a HCDR3 comprising an amino acid sequence as set forth in SEQ ID NO: 107;
xiv. a HCDR1 comprising an amino acid sequence as set forth in SEQ ID NO: 113, a HCDR2 comprising an amino acid sequence as set forth in SEQ ID NO: 114, and a HCDR3 comprising an amino acid sequence as set forth in SEQ ID NO: 115;
xv. a HCDR1 comprising an amino acid sequence as set forth in SEQ ID NO: 121, a HCDR2 comprising an amino acid sequence as set forth in SEQ ID NO: 122, and a HCDR3 comprising an amino acid sequence as set forth in SEQ ID NO: 123;
xvi. a HCDR1 comprising an amino acid sequence as set forth in SEQ ID NO: 129, a HCDR2 comprising an amino acid sequence as set forth in SEQ ID NO: 130, and a HCDR3 comprising an amino acid sequence as set forth in SEQ ID NO: 131;
xvii. a HCDR1 comprising an amino acid sequence as set forth in SEQ ID NO: 137, a HCDR2 comprising an amino acid sequence as set forth in SEQ ID NO: 138, and a HCDR3 comprising an amino acid sequence as set forth in SEQ ID NO: 139; or
xviii. a HCDR1 comprising an amino acid sequence as set forth in SEQ ID NO: 145, a HCDR2 comprising an amino acid sequence as set forth in SEQ ID NO: 146, and a HCDR3 comprising an amino acid sequence as set forth in SEQ ID NO: 147.

In some embodiments, the antibody or antigen-binding fragment thereof of the present disclosure comprises:
i. a LCDR1 comprising an amino acid sequence as set forth in SEQ ID NO: 20, a LCDR2 comprising an amino acid sequence as set forth in SEQ ID NO: 21, and a LCDR3 comprising an amino acid sequence as set forth in SEQ ID NO: 22;
ii. a LCDR1 comprising an amino acid sequence as set forth in SEQ ID NO: 28, a LCDR2 comprising an amino acid sequence as set forth in SEQ ID NO: 29, and a LCDR3 comprising an amino acid sequence as set forth in SEQ ID NO: 30;
iii. a LCDR1 comprising an amino acid sequence as set forth in SEQ ID NO: 36, a LCDR2 comprising an amino acid sequence as set forth in SEQ ID NO: 37, and a LCDR3 comprising an amino acid sequence as set forth in SEQ ID NO: 38;
iv. a LCDR1 comprising an amino acid sequence as set forth in SEQ ID NO: 44, a LCDR2 comprising an amino acid sequence as set forth in SEQ ID NO: 45, NO: 46;
v. a LCDR1 comprising an amino acid sequence as set forth in SEQ ID NO: 52, a LCDR2 comprising an amino acid sequence as set forth in SEQ ID NO: 53, and a LCDR3 comprising an amino acid sequence as set forth in SEQ ID NO: 54;
vi. a LCDR1 comprising an amino acid sequence as set forth in SEQ ID NO: 60, a LCDR2 comprising an amino acid sequence as set forth in SEQ ID NO: 61, and a LCDR3 comprising an amino acid sequence as set forth in SEQ ID NO: 62;
vii. a LCDR1 comprising an amino acid sequence as set forth in SEQ ID NO: 68, a LCDR2 comprising an amino acid sequence as set forth in SEQ ID NO: 69, and a LCDR3 comprising an amino acid sequence as set forth in SEQ ID NO: 70;
viii. a LCDR1 comprising an amino acid sequence as set forth in SEQ ID NO: 76, a LCDR2 comprising an amino acid sequence as set forth in SEQ ID NO: 77, and a LCDR3 comprising an amino acid sequence as set forth in SEQ ID NO: 78;
ix. a LCDR1 comprising an amino acid sequence as set forth in SEQ ID NO: 84, a LCDR2 comprising an amino acid sequence as set forth in SEQ ID NO: 85, and a LCDR3 comprising an amino acid sequence as set forth in SEQ ID NO: 86;
x. a LCDR1 comprising an amino acid sequence as set forth in SEQ ID NO: 92, a LCDR2 comprising an amino acid sequence as set forth in SEQ ID NO: 93, and a LCDR3 comprising an amino acid sequence as set forth in SEQ ID NO: 94;
xi. a LCDR1 comprising an amino acid sequence as set forth in SEQ ID NO: 100, a LCDR2 comprising an amino acid sequence as set forth in SEQ ID NO: 101, and a LCDR3 comprising an amino acid sequence as set forth in SEQ ID NO: 102;
xii. a LCDR1 comprising an amino acid sequence as set forth in SEQ ID NO: 108, a LCDR2 comprising an amino acid sequence as set forth in SEQ ID NO: 109, and a LCDR3 comprising an amino acid sequence as set forth in SEQ ID NO: 110;
xiii. a LCDR1 comprising an amino acid sequence as set forth in SEQ ID NO: 116, a LCDR2 comprising an amino acid sequence as set forth in SEQ ID NO: 117, and a LCDR3 comprising an amino acid sequence as set forth in SEQ ID NO: 118;
xiv. a LCDR1 comprising an amino acid sequence as set forth in SEQ ID NO: 124, a LCDR2 comprising an amino acid sequence as set forth in SEQ ID NO: 125, and a LCDR3 comprising an amino acid sequence as set forth in SEQ ID NO: 126;
xv. a LCDR1 comprising an amino acid sequence as set forth in SEQ ID NO: 132, a LCDR2 comprising an amino acid sequence as set forth in SEQ ID NO: 133, and a LCDR3 comprising an amino acid sequence as set forth in SEQ ID NO: 134;
xvi. a LCDR1 comprising an amino acid sequence as set forth in SEQ ID NO: 140, a LCDR2 comprising an amino acid sequence as set forth in SEQ ID NO: 141, and a LCDR3 comprising an amino acid sequence as set forth in SEQ ID NO: 142; or
xvii. a LCDR1 comprising an amino acid sequence as set forth in SEQ ID NO: 148, a LCDR2 comprising an amino acid sequence as set forth in SEQ ID NO: 149, and a LCDR3 comprising an amino acid sequence as set forth in SEQ ID NO: 150.

In some embodiments, the antibody or antigen-binding fragment thereof provided herein comprises:
i. a HCDR1 comprising an amino acid sequence as set forth in SEQ ID NO: 17, a HCDR2 comprising an amino acid sequence as set forth in SEQ ID NO: 18, a HCDR3 comprising an amino acid sequence as set forth in SEQ ID NO: 19, a LCDR1 comprising an amino acid sequence as set forth in SEQ ID NO: 20, a LCDR2 comprising an amino acid sequence as set forth in SEQ ID NO: 21, and a LCDR3 comprising an amino acid sequence as set forth in SEQ ID NO: 22;
ii. a HCDR1 comprising an amino acid sequence as set forth in SEQ ID NO: 25, a HCDR2 comprising an amino acid sequence as set forth in SEQ ID NO: 26, a HCDR3 comprising an amino acid sequence as set forth in SEQ ID NO: 27, a LCDR1 comprising an amino acid sequence as set forth in SEQ ID NO: 28, a LCDR2 comprising an amino acid sequence as set forth in SEQ ID NO: 29, and a LCDR3 comprising an amino acid sequence as set forth in SEQ ID NO: 30;
iii. a HCDR1 comprising an amino acid sequence as set forth in SEQ ID NO: 33, a HCDR2 comprising an amino acid sequence as set forth in SEQ ID NO: 34, a HCDR3 comprising an amino acid sequence as set forth in SEQ ID NO: 35, a LCDR1 comprising an amino acid sequence as set forth in SEQ ID NO: 36, a LCDR2 comprising an amino acid sequence as set forth in SEQ ID NO: 37, and a LCDR3 comprising an amino acid sequence as set forth in SEQ ID NO: 38;
iv. a HCDR1 comprising an amino acid sequence as set forth in SEQ ID NO: 41, a HCDR2 comprising an amino acid sequence as set forth in SEQ ID NO: 42, a HCDR3 comprising an amino acid sequence as set forth in SEQ ID NO: 43, a LCDR1 comprising an amino acid sequence as set forth in SEQ ID NO: 44, a LCDR2 comprising an amino acid sequence as set forth in SEQ ID NO: 45, NO: 46;
v. a HCDR1 comprising an amino acid sequence as set forth in SEQ ID NO: 41, a HCDR2 comprising an amino acid sequence as set forth in SEQ ID NO: 158, a HCDR3 comprising an amino acid sequence as set forth in SEQ ID NO: 43, a LCDR1 comprising an amino acid sequence as set forth in SEQ ID NO: 44, a LCDR2 comprising an amino acid sequence as set forth in SEQ ID NO: 45, and a LCDR3 comprising an amino acid sequence as set forth in SEQ ID NO: 46;
vi. a HCDR1 comprising an amino acid sequence as set forth in SEQ ID NO: 49, a HCDR2 comprising an amino acid sequence as set forth in SEQ ID NO: 50, a HCDR3 comprising an amino acid sequence as set forth in SEQ ID NO: 51, a LCDR1 comprising an amino acid sequence as set forth in SEQ ID NO: 52, a LCDR2 comprising an amino acid sequence as set forth in SEQ ID NO: 53, and a LCDR3 comprising an amino acid sequence as set forth in SEQ ID NO: 54;
vii. a HCDR1 comprising an amino acid sequence as set forth in SEQ ID NO: 57, a HCDR2 comprising an amino acid sequence as set forth in SEQ ID NO: 58, a HCDR3 comprising an amino acid sequence as set forth in SEQ ID NO: 59, a LCDR1 comprising an amino acid sequence as set forth in SEQ ID NO: 60, a LCDR2 comprising an amino acid sequence as set forth in SEQ ID NO: 61, and a LCDR3 comprising an amino acid sequence as set forth in SEQ ID NO: 62;
viii. a HCDR1 comprising an amino acid sequence as set forth in SEQ ID NO: 65, a HCDR2 comprising an amino acid sequence as set forth in SEQ ID NO: 66, a HCDR3 comprising an amino acid sequence as set forth in SEQ ID NO: 67, a LCDR1 comprising an amino acid sequence as set forth in SEQ ID NO: 68, a LCDR2 comprising an amino acid sequence as set forth in SEQ ID NO: 69, NO: 70;
ix. a HCDR1 comprising an amino acid sequence as set forth in SEQ ID NO: 73, a HCDR2 comprising an amino acid sequence as set forth in SEQ ID NO: 74, a HCDR3 comprising an amino acid sequence as set forth in SEQ ID NO: 75, a LCDR1 comprising an amino acid sequence as set forth in SEQ ID NO: 76, a LCDR2 comprising an amino acid sequence as set forth in SEQ ID NO: 77, and a LCDR3 comprising an amino acid sequence as set forth in SEQ ID NO: 78;
x. a HCDR1 comprising an amino acid sequence as set forth in SEQ ID NO: 81, a HCDR2 comprising an amino acid sequence as set forth in SEQ ID NO: 82, a HCDR3 comprising an amino acid sequence as set forth in SEQ ID NO: 83, a LCDR1 comprising an amino acid sequence as set forth in SEQ ID NO: 84, a LCDR2 comprising an amino acid sequence as set forth in SEQ ID NO: 85, and a LCDR3 comprising an amino acid sequence as set forth in SEQ ID NO: 86;
xi. a HCDR1 comprising an amino acid sequence as set forth in SEQ ID NO: 89, a HCDR2 comprising an amino acid sequence as set forth in SEQ ID NO: 90, a HCDR3 comprising an amino acid sequence as set forth in SEQ ID NO: 91, a LCDR1 comprising an amino acid sequence as set forth in SEQ ID NO: 92, a LCDR2 comprising an amino acid sequence as set forth in SEQ ID NO: 93, and a LCDR3 comprising an amino acid sequence as set forth in SEQ ID NO: 94;
xii. a HCDR1 comprising an amino acid sequence as set forth in SEQ ID NO: 97, a HCDR2 comprising an amino acid sequence as set forth in SEQ ID NO: 98, a HCDR3 comprising an amino acid sequence as set forth in SEQ ID NO: 99, a LCDR1 comprising an amino acid sequence as set forth in SEQ ID NO: 100, a LCDR2 comprising an amino acid sequence as set forth in SEQ ID NO: 101, and a LCDR3 comprising an amino acid sequence as set forth in SEQ ID NO: 102;
xiii. a HCDR1 comprising an amino acid sequence as set forth in SEQ ID NO: 105, a HCDR2 comprising an amino acid sequence as set forth in SEQ ID NO: 106, a HCDR3 comprising an amino acid sequence as set forth in SEQ ID NO: 107, a LCDR1 comprising an amino acid sequence as set forth in SEQ ID NO: 108, a LCDR2 comprising an amino acid sequence as set forth in SEQ ID NO: 109, and a LCDR3 comprising an amino acid sequence as set forth in SEQ ID NO: 110;
xiv. a HCDR1 comprising an amino acid sequence as set forth in SEQ ID NO: 113, a HCDR2 comprising an amino acid sequence as set forth in SEQ ID NO: 114, a HCDR3 comprising an amino acid sequence as set forth in SEQ ID NO: 115, a LCDR1 comprising an amino acid sequence as set forth in SEQ ID NO: 116, a LCDR2 comprising an amino acid sequence as set forth in SEQ ID NO: 117, and a LCDR3 comprising an amino acid sequence as set forth in SEQ ID NO: 118;
xv. a HCDR1 comprising an amino acid sequence as set forth in SEQ ID NO: 121, a HCDR2 comprising an amino acid sequence as set forth in SEQ ID NO: 122, a HCDR3 comprising an amino acid sequence as set forth in SEQ ID NO: 123, a LCDR1 comprising an amino acid sequence as set forth in SEQ ID NO: 124, a LCDR2 comprising an amino acid sequence as set forth in SEQ ID NO: 125, and a LCDR3 comprising an amino acid sequence as set forth in SEQ ID NO: 126;
xvi. a HCDR1 comprising an amino acid sequence as set forth in SEQ ID NO: 129, a HCDR2 comprising an amino acid sequence as set forth in SEQ ID NO: 130, a HCDR3 comprising an amino acid sequence as set forth in SEQ ID NO: 131, a LCDR1 comprising an amino acid sequence as set forth in SEQ ID NO: 132, a LCDR2 comprising an amino acid sequence as set forth in SEQ ID NO: 133, and a LCDR3 comprising an amino acid sequence as set forth in SEQ ID NO: 134;
xvii. a HCDR1 comprising an amino acid sequence as set forth in SEQ ID NO: 137, a HCDR2 comprising an amino acid sequence as set forth in SEQ ID NO: 138, a HCDR3 comprising an amino acid sequence as set forth in SEQ ID NO: 139, a LCDR1 comprising an amino acid sequence as set forth in SEQ ID NO: 140, a LCDR2 comprising an amino acid sequence as set forth in SEQ ID NO: 141, and a LCDR3 comprising an amino acid sequence as set forth in SEQ ID NO: 142; or
xviii. a HCDR1 comprising an amino acid sequence as set forth in SEQ ID NO: 145, a HCDR2 comprising an amino acid sequence as set forth in SEQ ID NO: 146, a HCDR3 comprising an amino acid sequence as set forth in SEQ ID NO: 147, a LCDR1 comprising an amino acid sequence as set forth in SEQ ID NO: 148, a LCDR2 comprising an amino acid sequence as set forth in SEQ ID NO: 149, and a LCDR3 comprising an amino acid sequence as set forth in SEQ ID NO: 150.

In some embodiments, the antibody or antigen-binding fragment thereof provided herein comprises a VH region having an amino acid sequence as set forth in SEQ ID NOs: 23, 31, 39, 47, 55, 63, 71, 79, 87, 95, 103, 111, 119, 127, 135, 143, 151, 154 or 156, or a homologous sequence thereof having at least 80% sequence identity to SEQ ID NOs: 23, 31, 39, 47, 55, 63, 71, 79, 87, 95, 103, 111, 119, 127, 135, 143, 151, 154 or 156.

In some embodiments, the antibody or antigen-binding fragment thereof provided herein comprises a VL region having an amino acid sequence as set forth in SEQ ID NOs: 24, 32, 40, 48, 56, 64, 72, 80, 88, 96, 104, 112, 120, 128, 136, 144, 152, 155 or 157, or a homologous sequence thereof having at least 80% sequence identity to SEQ ID NOs: 24, 32, 40, 48, 56, 64, 72, 80, 88, 96, 104, 112, 120, 128, 136, 144, 152, 155 or 157.

In some embodiments, the antibody or antigen-binding fragment thereof provided herein comprises a VH/VL amino acid sequence pair selected from the group consisting of SEQ ID NOs: 23/24, 31/32, 39/40, 47/48, 55/56, 63/64, 71/72, 79/80, 87/88, 95/96, 103/104, 111/112, 119/120, 127/128, 135/136, 143/144, 151/152, 154/155, and 156/157.

In some embodiments, the antibody or antigen-binding fragment thereof provided herein further comprises one or more amino acid residue substitutions or modifications yet retains binding affinity to LILRB4. In some embodiments, at least one of the substitutions or modifications is in one or more of the CDR sequences of the VH region or VL region. In some embodiments, at least one of the substitutions or modifications is in one or more of the non-CDR sequences of the VH region or VL region. In some embodiments, the antibody or antigen-binding fragment thereof provided herein further comprises one or more non-natural amino acid (NNAA) substitution. In some embodiments, the NNAA is capable of being conjugated.

In some embodiments, the antibody or antigen-binding fragment thereof provided herein has one or more binding properties to LILRB4 selected from the group consisting of:
i. being capable of specifically binding to human LILRB4 as measured by FACS assay;
ii. being capable of specifically binding to human LILRB4 and human LILRB3 as measured by FACS assay;
iii. being capable of specifically binding to human LILRB4 and cynomolgus LILRB4 as measured by FACS assay;
iv. being with potent ADCP effect on a human AML cell line;
v. being with potent ADCC effect on a human AML cell line;
vi. being capable of blocking the LILRB4-fibronectin interaction to reprogram tolerogenic dendritic cells to be mature dendritic cells which stimulate T cell activation as measured by FcγR stimulation assay;
vii. being capable of inducing TNF-α production;
viii. being capable of reprograming tolerogenic DC to activate T cells;
ix. being capable of reversing macrophage-mediated T cell suppression;
x. being capable of reversing THP-1-mediated T cell suppression; and
xi. being capable of potentiating CD8⁺ T cell-mediated cytotoxicity on THP-1 cells.

In another aspect, the present disclosure provides an antibody or antigen-binding fragment thereof, which competes for binding to LILRB4 with the antibody or antigen-binding fragment thereof as described above.

In some embodiments, the antibody or antigen-binding fragment thereof provided herein is a chimeric, a humanized or a human antibody or an antigen-binding fragment thereof.

In some embodiments, the antibody or antigen-binding fragment thereof provided herein is a labeled antibody, a bivalent antibody, an anti-idiotypic antibody or a fusion protein.

In some embodiments, the antibody or antigen-binding fragment thereof provided herein is a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a camelized single domain antibody, a nanobody, a domain antibody, or a bivalent domain antibody.

In some embodiments, the antibody or antigen-binding fragment thereof provided herein further comprises an Fc region. In some embodiments, the Fc region is an Fc region of human immunoglobulin (Ig). In some embodiments, the Fc region is an Fc region of human IgG. In some embodiments, the Fc region is derived from human IgG1, IgG2, IgG3, or IgG4. In some embodiments, the Fc region comprises an amino acid sequence as set forth in SEQ ID NO: 153.

In some embodiments, the light chain of the antibody or antigen-binding fragment thereof provided herein is a λ light chain or a κ light chain.

In some embodiments, the antibody or antigen-binding fragment thereof provided herein is a bispecific or multi-specific antibody or an antigen-binding fragment thereof. In some embodiments, the antibody or antigen-binding fragment thereof provided herein is capable of specifically binding to one or more additional antigens other than LILRB4, or a second epitope on LILRB4. In some embodiments, the one or more additional antigens other than LILRB4 are selected from the group consisting of CD3, CD16a, CD33, CD38, CD45, CD123, CD146, CD228, CLL-1, Flt3, TAF1, TgPRF, HVCN1, IL-6R, IL-11R, IL17A, IL-23R, IL-33, ILDR2, LAP, TSLP, TREM-1, ANGPT2, APOE, IFNAR, CypA, DOG-1, NKp30, CSF-1R, CCR2, LRRC15, mesothelin, Dickkopf2, DLL3, HER-2, C10orf54, TrkA, MEKK1, KRAS, ERK, XPO1, mTORC1/2, PAK4, NAMPT, ATR, EGFR, FGFR, VEGF, c-MET, Her2, Her3, CTLA4, GITA, CD112R, CD2, CD7, CD16, CD19, CD20, CD24, CD27, CD30, CD34, CD37, CD39, CD70, CD73, CD83, CD28, CD80 (B7-1), CD86 (B7-2), CD40, CD40L (CD154), CD47, SIRPα, CD122, CD137, CD137L, OX40 (CD134), OX40L (CD252), BCMA (e.g., BCMA02), PSMA, CLDN18 (e.g., CLDN18.2), NKG2C, 4-1BB, LIGHT, PVRIG, SLAMF7, HVEM, BAFFR, ICAM-1, 2B4, LFA-1, GITR, ICOS (CD278), ICOSLG (CD275), LAG3 (CD223), A2AR, B7-H3 (CD276), B7-H4 (VTCN1), B7-H5, BTLA (CD272), BTLA, CD160, CTLA-4 (CD152), GPRC5D, IDO1, IDO2, ILT3, TDO, KIR, LAIR-1, NOX2, PD-1, PD-L1, PD-L2, TIM-3, VISTA, SIGLEC-7 (CD328), SIGLEC-9 (CD329), SIGLEC-15, TIGIT, PVR (CD155), LILRB2, LILRB3, FLT3, FLT3L, TLR3, CLEC9A, DEC-205, STING, IL-12, IDO, and TGFβ.

In some embodiments, the antibody or antigen-binding fragment thereof provided herein is linked to one or more conjugate moieties. In some embodiments, the conjugate moiety comprises a clearance-modifying agent, a chemotherapeutic agent, a toxin, a radioactive isotope, a lanthanide, a detectable label, a DNA-alkylator, a topoisomerase inhibitor, a tubulin-binder, a purification moiety or other anticancer drugs. In some embodiments, the conjugate moiety is covalently attached either directly or via a linker.

In another aspect, the present disclosure provides a chimeric antigen receptor comprising the antibody or antigen-binding fragment thereof provided herein, a transmembrane region and an intracellular signal region. In some embodiments, the transmembrane region comprises a transmembrane region of CD3, CD4, CD8 or CD28. In some embodiments, the intracellular signal region is selected from the group consisting of: an intracellular signal regions sequence of CD3, FcγRI, CD27, CD28, CD137, CD134, MyD88, CD40, CD278, TLRs, or a combination thereof. In some embodiments, the antigen-binding fragment of the chimeric antigen receptor is a scFv. In some embodiments, the chimeric antigen receptor is grafted onto an allogeneic cell, an autologous cell or a xenogeneic cell. In some embodiments, the chimeric antigen receptor is grafted onto an immune effector cell. In some embodiments, the chimeric antigen receptor is grafted onto a T cell, a natural killer cell, a macrophage cell, or a tumor-infiltrating lymphocyte.

In another aspect, the present disclosure provides a pharmaceutical composition comprising the antibody or antigen-binding fragment thereof, and/or the chimeric antigen receptor of the present disclosure, and one or more pharmaceutically acceptable carriers.

In another aspect, the present disclosure provides an isolated polynucleotide encoding the antibody or antigen-binding fragment thereof and/or the chimeric antigen receptor of the present disclosure.

In another aspect, the present disclosure provides a vector comprising the isolated polynucleotide of the present disclosure.

In another aspect, the present disclosure provides a host expression system comprising the vector of the present disclosure or having the polynucleotide of the present disclosure integrated into genome thereof. In some embodiments, the host expression system of the present disclosure is a microorganism, a yeast, or a mammalian cell. In some embodiments, the microorganism is selected from the group consisting of E. coli and B. subtilis. In some embodiments, the yeast is Saccharomyces. In some embodiments, the mammalian cell is selected from the group consisting of COS, CHO-S, CHO-K1, HEK-293, and 3T3 cells.

In another aspect, the present disclosure provides a virus comprising the vector of the present disclosure.

In another aspect, the present disclosure provides a kit comprising the antibody or antigen-binding fragment thereof of the present disclosure and/or the chimeric antigen receptor of the present disclosure and/or the pharmaceutical composition of the present disclosure, and a second therapeutic agent.

In another aspect, the present disclosure provides a method of expressing the antibody or antigen-binding fragment thereof of the present disclosure and/or the chimeric antigen receptor of the present disclosure, comprising culturing the host expression system of the present disclosure under the condition at which the antibody or antigen-binding fragment thereof of the present disclosure or the chimeric antigen receptor of the present disclosure is expressed.

In another aspect, the present disclosure provides a method of treating, preventing or alleviating a disease, disorder or condition in a subject, comprising administering to the subject a therapeutically effective amount of the antibody or antigen-binding fragment thereof and/or the chimeric antigen receptor and/or the pharmaceutical composition of the present disclosure.

In another aspect, the present disclosure provides use of the antibody or antigen-binding fragment thereof and/or the chimeric antigen receptor and/or the pharmaceutical composition of the present disclosure in the manufacture of a medicament for treating a LILRB4-related disease, disorder or condition in a subject.

In another aspect, the present disclosure provides use of the antibody or antigen-binding fragment thereof and/or the chimeric antigen receptor and/or the pharmaceutical composition of the present disclosure in the manufacture of a diagnostic reagent for diagnosing a LILRB4-related disease, disorder or condition.

In some embodiments, the disease, disorder or condition is immune disease, inflammatory disease, cancer or neurological disease. In some embodiments, the cancer is a solid tumor or hematologic tumor. In some embodiments, the disease, disorder or condition is a LILRB4-expressing B cell cancer. In some embodiments, the disease, disorder or condition is selected from the group consisting of Kawasaki disease, T. gondii, multiple sclerosis, systematic Lupus erythematosus, lung cancer (e.g., non-small-cell lung cancer (NSCLC), small cell lung cancer (SCLC), adenocarcinoma of the lung, squamous cell carcinoma of the lung, Lewis lung carcinoma, or radiation therapy resistant Lewis lung carcinoma), peritoneal cancer, carcinoid cancer, bone cancer, pancreatic cancer, primitive neuroectodermal tumor, skin cancer, gallbladder cancer, cancer of the head or neck, squamous cell cancer, uterine cancer, ovarian cancer, rectal cancer, prostate cancer, bladder cancer (e.g., urothelial cancer), cancer of the anal region (e.g., anal squamous cell carcinoma), gastric or stomach cancer (e.g., gastrointestinal cancer), esophageal cancer, colon cancer, breast cancer, uterine cancer, liver cancer (e.g., hepatoblastoma, hepatocellular carcinoma/hepatoma, or hepatic carcinoma), cholangiocarcinoma, sarcoma, colorectal cancer, carcinoma of the fallopian tubes, salivary gland carcinoma, carcinoma of the cervix, endometrial or uterine carcinoma, osteosarcoma, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, cancer of the nasopharynx, sarcoma of soft tissue, polycythemia vera, cancer of the urethra, cancer of the penis, cancer of the kidney or ureter (e.g., rhabdoid tumor of the kidney), cutaneous T-cell lymphoma, medulloblastoma, nephroblastoma, myelodysplastic syndrome, chronic and non-chronic myeloproliferative disorder, choroid plexus papilloma, renal cell carcinoma, carcinoma of the renal pelvis, neoplasms of the central nervous system (CNS), soft tissue sarcoma (e.g., rhabdomyosarcoma, fibrosarcoma, Kaposi's sarcoma), spinal axis tumors, glioma (e.g., ependymoma, astrocytoma, anaplastic astrocytoma, oligodendroglioma, eye cancer (e.g., retinoblastoma), brain stem glioma, or mixed glioma such as oligoastrocytoma), brain tumor (e.g., glioblastoma/glioblastoma multiforme (GBM), non-glioblastoma brain tumor, or meningioma), melanoma (e.g., cutaneous or intraocular melanoma), thrombocythemia, mesothelioma, mycosis fungoides, Sezary syndrome, idiopathic myelofibrosis, solitary plasmacytoma, vestibular schwannoma, Ewing's sarcoma, chondrosarcoma, MYH associated polyposis, pituitary adenoma, pediatric cancers such as pediatric sarcomas (e.g., neuroblastoma, rhabdomyosarcoma, and osteosarcoma), hematological cancer, lymphoma, Hodgkin's lymphoma, non-Hodgkin's lymphoma, leukemia (e.g., lymphocytic/lymphoblastic leukemia), chronic or acute leukemia, mast cell leukemia, lymphocytic lymphomas, primary CNS lymphoma, chronic lymphocytic leukemia (CLL), acute lymphocytic leukemia (ALL), chronic myeloid leukemia (CML), acute myeloid leukemia (AML), chronic myelomonocytic leukemia (CMML), chronic lymphoblastic leukemia, acute lymphoblastic leukemia, hairy cell leukemia (HCL), Burkitt's lymphoma (BL), multiple myeloma (e.g., relapsed or refractory multiple myeloma), T or B cell lymphoma, mantle cell lymphoma (MCL) (e.g., relapsed or refractory mantle cell lymphoma), malignant melanoma, diffuse large B cell lymphoma (DLBCL), DLBCL that results from follicular lymphoma, high-grade B-cell lymphoma, primary mediastinal large B-cell lymphoma, follicular lymphoma (FL), and primary mediastinal B-cell lymphoma. In some embodiments, the disease, disorder or condition is acute myeloid leukemia. In some embodiments, the disease, disorder or condition is chronic myelomonocytic leukemia.

In some embodiments, the subject is human.

In some embodiments, the administration is through a parenteral route comprising subcutaneous, intraperitoneal, intravenous, intramuscular, or intradermal injection; or a non-parenteral route comprising transdermal, oral, intranasal, intraocular, sublingual, rectal, or topical.

In some embodiments, the method of treating, preventing or alleviating a disease, disorder or condition in a subject further includes administering to the subject in need thereof an additional therapeutic agent. In some embodiments, the additional therapeutic agent is selected from the group consisting of: an active agent, an imaging agent, a cytotoxic agent, and angiogenesis inhibitor, a kinase inhibitor, a co-stimulation molecule agonist, a co-inhibition molecule blocker, an adhesion molecule blocker, an anti-cytokine antibody or functional fragment thereof, a detectable label or reporter, an antimicrobial, a gene editing agent, a beta agonist, an viral RNA inhibitor, a polymerase inhibitor, an interferon, and a microRNA. In some embodiments, the additional therapeutic agent is administered to the subject in need before, after or simultaneously with the antibody or antigen-binding fragment thereof and/or the pharmaceutical composition and/or the chimeric antigen receptor of the present disclosure.

In another aspect, the present disclosure provides a method of modulating LILRB4 activity in a LILRB4-expressing cell, comprising exposing the LILRB4-expressing cell to the antibody or antigen-binding fragment thereof and/or the pharmaceutical composition and/or the chimeric antigen receptor of the present disclosure.

In some embodiments, the LILRB4-expressing cell is a dendritic cell, monocyte, macrophage, B cell, Treg, progenitor mast cell, endothelial cell, or osteoclast.

In another aspect, the present disclosure provides a method of inducing phagocytosis of a target cell in vivo or in vitro, comprising exposing the target cell to the antibody or antigen-binding fragment thereof and/or the pharmaceutical composition and/or the chimeric antigen receptor of the present disclosure. In some embodiments, the target cell is an antigen presenting cell, a cancer cell or a cell infected by a pathogen.

In another aspect, the present disclosure provides a method of inducing TNF-α production, comprising exposing the tolerogenic dendritic cell to the antibody or antigen-binding fragment thereof and/or the pharmaceutical composition and/or the chimeric antigen receptor of the present disclosure.

In another aspect, the present disclosure provides a method of reprogramming a tolerogenic dendritic cell to a mature dendritic cell, comprising exposing the tolerogenic dendritic cell to the antibody or antigen-binding fragment thereof and/or the pharmaceutical composition and/or the chimeric antigen receptor of the present disclosure.

In another aspect, the present disclosure provides a method of detecting presence or amount of LILRB4 in a sample, comprising contacting the sample with the antibody or antigen-binding fragment thereof and/or the pharmaceutical composition and/or the chimeric antigen receptor of the present disclosure, and determining the presence or the amount of LILRB4 in the sample.

In another aspect, the present disclosure provides a method of diagnosing a LILRB4 related disease or condition in a subject, comprising: a) obtaining a sample from the subject; b) contacting the sample obtained from the subject with the antibody or antigen-binding fragment thereof and/or the pharmaceutical composition and/or the chimeric antigen receptor of the present disclosure; c) determining presence or amount of LILRB4 in the sample; and d) correlating the presence or the amount of LILRB4 to existence or status of the LILRB4 related disease or condition in the subject.

In another aspect, the present disclosure provides a kit comprising the antibody or antigen-binding fragment thereof and/or the pharmaceutical composition and/or the chimeric antigen receptor of the present disclosure, useful in detecting LILRB4, optionally recombinant LILRB4, LILRB4 expressed on cell surface, or LILRB4-expressing cells.

### BRIEF DESCRIPTION OF THE DRAWINGS

FIG. 1 shows the binding affinities of the selected chimeric antibodies and benchmark antibodies (IO-202 and NGM831, which were generated as human IgG1 chimeric antibodies) on 293F-hLILRB4 cell as measured by FACS assay.

FIGS. 2A-B show the binding affinities of ch43-D12-F3-G11 and ch42-C8-A12-F3-D11 on 293F-hLILRB3 cells (FIG. 2A) and 293F-hLILRB4 cells (FIG. 2B) as measured by FACS assay.

FIGS. 3A-B show the binding affinity of ch44-F10-B6-D4 on CHO-S-cynoLILRB4 cells (FIG. 3A) and 293F-hLILRB4 cells (FIG. 3B) as measured by FACS assay.

FIG. 4 shows the FACS analysis of LILRB4 expression in different human cancer cell lines.

FIG. 5 shows the ADCC effect of antibody ch2-H1-D7-E5-D5 compared with the benchmark antibodies IO-202 and NGM831 on THP-1 cells.

FIG. 6 shows the ADCP effect of antibody ch2-H1-D7-E5-D5 compared with the benchmark antibody IO-202 on MV-4-11 cells.

FIGS. 7A-D show the in vivo efficacy test results of selected chimeric antibody in the treatment of radiation therapy resistant Lewis lung carcinoma (FIG. 7A: tumor growth inhibition; FIG. 7C: body weight) and EL4-LILRB4 lymphoma (FIG. 7B: tumor growth inhibition; FIG. 7D: body weight) in female hLILRB1/hLILRB4 transgenic mice.

FIGS. 8A-F show the binding affinities of humanized antibodies h2-H1-D7-E5-D5 and h8-B3-F6-H8 to LILRB4 compared with the benchmark antibodies IO-202 and NGM831.

FIG. 9 shows the mixed lymphocyte reaction (MLR) results of humanized antibodies h2-H1-D7-E5-D5 and h8-B3-F6-H8 compared with the benchmark antibodies IO-202 and NGM831.

FIG. 10 shows the macrophage and T cell coculture assay results of humanized antibodies h2-H1-D7-E5-D5 and h8-B3-F6-H8 compared with the benchmark antibodies IO-202 and NGM831.

FIGS. 11A-D show the THP-1 and T cell coculture assay results of chimeric antibodies ch2-H1-D7-E5-D5 and ch8-B3-F6-H8 compared with the benchmark antibody IO-202.

FIG. 12 shows the cytotoxic CD8⁺ T cell killing assay results of humanized antibodies h2-H1-D7-E5-D5 and h8-B3-F6-H8 compared with the benchmark antibody IO-202 on THP-1 cells.

FIGS. 13A-B show the ADCC effect (FIG. 13A) and ADCP effect (FIG. 13B) of humanized antibody h2-H1-D7-E5-D5 compared with the benchmark antibody IO-202 on THP-1 cells.

FIGS. 14A-B show the in vivo efficacy test results of humanized antibodies h2-H1-D7-E5-D5 and h8-B3-F6-H8 in the treatment of EL4-LILRB4 lymphoma (FIG. 14A: tumor growth inhibition; FIG. 14B: body weight) in female hLILRB1/hLILRB4 transgenic mice.

### DETAILED DESCRIPTION OF THE INVENTION

The following description of the disclosure is merely intended to illustrate various embodiments of the disclosure. As such, the specific modifications discussed are not to be construed as limitations on the scope of the disclosure. It will be apparent to a person skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entireties.

### Definitions

The term “antibody” as used herein includes any immunoglobulin, monoclonal antibody, polyclonal antibody, multivalent antibody, bivalent antibody, monovalent antibody, multi-specific antibody, or bispecific antibody that binds to a specific antigen. A native intact antibody comprises two heavy (H) chains and two light (L) chains. Mammalian heavy chains are classified as alpha, delta, epsilon, gamma, and mu, each heavy chain comprises a variable region (VH) and a first, second, third, and optionally fourth constant region (CH1, CH2, CH3, CH4 respectively); mammalian light chains are classified as λ or κ, while each light chain comprises a variable region (VL) and a constant region. The antibody has a “Y” shape, with the stem of the Y comprising the second and third constant regions of two heavy chains bound together via disulfide bonding. Each arm of the Y includes the variable region and first constant region of a single heavy chain bound to the variable and constant regions of a single light chain. The variable regions of the light and heavy chains are responsible for antigen binding. The variable regions in both chains generally contain three highly variable loops called the complementarity determining regions (CDRs) (light chain CDRs including LCDR1, LCDR2, and LCDR3, heavy chain CDRs including HCDR1, HCDR2, HCDR3). The three CDRs are interposed between flanking stretches known as framework regions (FRs) (light chain FRs including LFR1, LFR2, LFR3, and LFR4, heavy chain FRs including HFR1, HFR2, HFR3, and HFR4), which are more highly conserved than the CDRs and form a scaffold to support the highly variable loops. The constant regions of the heavy and light chains are not involved in antigen-binding, but exhibit various effector functions. Antibodies are assigned to classes based on the amino acid sequences of the constant regions of their heavy chains. The five major classes or isotypes of antibodies are IgA, IgD, IgE, IgG, and IgM, which are characterized by the presence of alpha, delta, epsilon, gamma, and mu heavy chains, respectively. Several of the major antibody classes are divided into subclasses such as IgG1 (gamma1 heavy chain), IgG2 (gamma2 heavy chain), IgG3 (gamma3 heavy chain), IgG4 (gamma4 heavy chain), IgA1 (alpha1 heavy chain), or IgA2 (alpha2 heavy chain).

In certain embodiments, the antibody provided herein encompasses any antigen-binding fragments thereof. The term “antigen-binding fragment” as used herein refers to an antibody fragment formed from a portion of an antibody comprising one or more (e.g., 1, 2, 3, 4, 5, or 6) CDRs, or any other antibody fragment that binds to an antigen but does not comprise an intact native antibody structure. Examples of antigen-binding fragments include, without limitation, a diabody, a Fab, a Fab′, a F(ab′) 2, a Fd, an Fv fragment, a disulfide stabilized Fv fragment (dsFv), a (dsFv) 2, a bispecific dsFv (dsFv-dsFv′), a disulfide stabilized diabody (ds diabody), a single-chain antibody molecule (scFv), an scFv dimer (bivalent diabody), a bispecific antibody, a multi-specific antibody, a camelized single domain antibody, a nanobody, a domain antibody, or a bivalent domain antibody. An antigen-binding fragment is capable of binding to the same antigen or epitope to which the parent antibody binds.

“Fab” with regard to an antibody refers to that portion of the antibody consisting of a single light chain (both variable and constant regions) bound to the variable region and first constant region of a single heavy chain by a disulfide bond.

“Fab′” refers to a Fab fragment that includes a portion of the hinge region. “F(ab′)₂” refers to a dimer of Fab′.

“Fc” with regard to an antibody (e.g., of IgG, IgA, or IgD isotype) refers to that portion of the antibody consisting of the second and third constant domains of a first heavy chain bound to the second and third constant domains of a second heavy chain via disulfide bonding. Fc with regard to antibody of IgM and IgE isotype further comprises a fourth constant domain. The Fc portion of the antibody is responsible for various effector functions such as antibody-dependent cell-mediated cytotoxicity (ADCC), and complement dependent cytotoxicity (CDC), but does not function in antigen binding.

“Fv” with regard to an antibody refers to the smallest fragment of the antibody to bear the complete antigen binding site. An Fv fragment consists of the variable region of a single light chain bound to the variable region of a single heavy chain.

“Single-chain Fv antibody” or “scFv” refers to an engineered antibody consisting of a light chain variable region and a heavy chain variable region connected to one another directly or via a linker (e.g., a peptide sequence) (Huston J S et al., Proc Natl Acad Sci USA, 85: 5879(1988)).

“Single-chain Fv-Fc antibody” or “scFv-Fc” refers to an engineered antibody consisting of a scFv connected to the Fc region of an antibody.

“Camelized single domain antibody”, “heavy chain antibody”, or “HCAb” refers to an antibody that contains two VH domains and no light chains (Riechmann L. and Muyldermans S., J Immunol Methods. December 10; 231(1-2):25-38 (1999); Muyldermans S., J Biotechnol. June; 74(4):277-302 (2001); WO94/04678; WO94/25591; U.S. Pat. No. 6,005,079). Heavy chain antibodies were originally derived from Camelidae (camels, dromedaries, and llamas). Although devoid of light chains, camelized antibodies have an authentic antigen-binding repertoire (Hamers-Casterman C. et al., Nature. June 3; 363(6428):446-8 (1993); Nguyen V K. et al., Immunogenetics. April; 54 (1): 39-47 (2002); Nguyen V K. et al., Immunology. May; 109(1):93-101 (2003)). The variable domain of a heavy chain antibody (VHH domain) represents the smallest known antigen-binding unit generated by adaptive immune responses (Koch-Nolte F. et al., FASEB J. November; 21(13):3490-8. Epub 2007 Jun. 15 (2007)).

A “nanobody” refers to an antibody fragment that consists of a VHH domain from a heavy chain antibody and two constant domains, CH2 and CH3.

A “diabody” or “dAb” includes small antibody fragments with two antigen-binding sites, wherein the fragments comprise a VH domain connected to a VL domain in the same polypeptide chain (VH-VL or VL-VH) (see, e.g., Holliger P. et al., Proc Natl Acad Sci USA. July 15; 90 (14): 6444-8 (1993); EP404097; WO93/11161). By using a linker that is too short to allow pairing between the two domains on the same chain, the domains are forced to pair with the complementary domains of another chain, thereby creating two antigen-binding sites. The antigen-binding sites may target the same or different antigens (or epitopes). In certain embodiments, a “bispecific ds diabody” is a diabody target two different antigens (or epitopes).

A “domain antibody” refers to an antibody fragment containing only the variable region of a heavy chain or the variable region of a light chain. In certain instances, two or more VH domains are covalently joined with a peptide linker to create a bivalent or multivalent domain antibody. The two VH domains of a bivalent domain antibody may target the same or different antigens.

The term “valent” as used herein refers to the presence of a specified number of antigen binding sites in a given molecule. The term “monovalent” refers to an antibody or an antigen-binding fragment having only one single antigen-binding site; and the term “multivalent” refers to an antibody or antigen-binding fragment having multiple antigen-binding sites. As such, the terms “bivalent”, “tetravalent”, and “hexavalent” denote the presence of two antigen-binding sites, four antigen-binding sites, and six antigen-binding sites, respectively, in an antigen-binding molecule. In some embodiments, the antibody or antigen-binding fragment thereof is bivalent.

As used herein, a “bispecific” antibody refers to an artificial antibody which has fragments derived from two different monoclonal antibodies and is capable of binding to two different epitopes. The two epitopes may present on the same antigen, or they may present on two different antigens.

As used herein, a “multi-specific” antibody refers to an antibody that specifically binds to at least two distinct antigens or at least two distinct epitopes within the same antigen. Multi-specific antibody may bind for example two, three, four, five or more distinct antigens or distinct epitopes within the same antigen.

In certain embodiments, an “scFv dimer” is a bivalent diabody or bispecific scFv (BsFv) comprising VH-VL (linked by a peptide linker) dimerized with another VH-VL moiety such that VH's of one moiety coordinate with the VL's of the other moiety and form two binding sites which can target the same antigens (or epitopes) or different antigens (or epitopes). In other embodiments, an “scFv dimer” is a bispecific diabody comprising VH1-VL2 (linked by a peptide linker) associated with VL1-VH2 (also linked by a peptide linker) such that VH1 and VL1 coordinate and VH2 and VL2 coordinate and each coordinated pair has a different antigen specificity.

A “dsFv” refers to a disulfide-stabilized Fv fragment that the linkage between the variable region of a single light chain and the variable region of a single heavy chain is a disulfide bond. In some embodiments, a “(dsFv) 2” or “(dsFv-dsFv′)” comprises three peptide chains: two VH moieties linked by a peptide linker (e.g., a long flexible linker) and bound to two VL moieties, respectively, via disulfide bridges. In some embodiments, dsFv-dsFv′ is bispecific in which each disulfide paired heavy and light chain has a different antigen specificity.

The term “chimeric” as used herein, means an antibody or antigen-binding fragment, having a portion of heavy and/or light chain derived from one species, and the rest of the heavy and/or light chain derived from a different species. In an illustrative example, a chimeric antibody may comprise a constant region derived from human and a variable region from a non-human animal, such as from mouse. In some embodiments, the non-human animal is a mammal, for example, a mouse, a rat, a rabbit, a goat, a sheep, a guinea pig, or a hamster.

The term “humanized” as used herein means that the antibody or antigen-binding fragment comprises CDRs derived from non-human animals, FR regions derived from human, and when applicable, the constant regions derived from human. The CDRs of humanized antibodies provided in the present disclosure may contain mutation(s) compared to the CDRs of their parent antibodies.

The term “affinity” as used herein refers to the strength of non-covalent interaction between an immunoglobulin molecule (i.e., antibody) or antigen-binding fragment thereof and an antigen.

An antibody or antigen-binding fragment thereof that “specifically binds” or “specific binding” to a target (e.g., an epitope) is a term well understood in the art, and methods to determine such specific binding are also well known in the art. A molecule is said to exhibit “specific binding” if it reacts or associates more frequently, more rapidly, with greater duration and/or with greater affinity with a particular cell or substance than it does with alternative cells or substances. An antibody “specifically binds” to a target if it binds with greater affinity, avidity, more readily, and/or with greater duration than it binds to other substances. For example, an antibody that specifically binds to a LILRB4 epitope is an antibody that binds this LILRB4 epitope with greater affinity, avidity, more readily, and/or with greater duration than it binds to other LILRB4 epitopes or non-LILRB4 epitopes. It is also understood by reading this definition that, for example, an antibody (or moiety or epitope) that specifically binds to a first target may or may not specifically bind to a second target. As such, “specific binding” or “specifically bind” does not necessarily require (although it can include) exclusive binding. Generally, but not necessarily, reference to binding means specific binding.

The ability to “compete for binding to LILRB4” as used herein refers to the ability of a first antibody or antigen-binding fragment to inhibit the binding interaction between LILRB4 and a second anti-LILRB4 antibody to any detectable degree. In certain embodiments, an antibody or antigen-binding fragment that competes for binding to LILRB4 inhibits the binding interaction between LILRB4 and a second anti-LILRB4 antibody by at least 85%, or at least 90%. In certain embodiments, this inhibition may be greater than 95%, or greater than 99%.

The term “epitope” as used herein refers to the specific group of atoms or amino acids on an antigen to which an antibody binds. Two antibodies may bind the same or a closely related epitope within an antigen if they exhibit competitive binding for the antigen. An epitope can be linear or conformational (i.e., including amino acid residues spaced apart). For example, if an antibody or antigen-binding fragment blocks binding of a reference antibody to the antigen by at least 85%, or at least 90%, or at least 95%, then the antibody or antigen-binding fragment may be considered to bind the same/closely related epitope as the reference antibody.

The term “amino acid” as used herein refers to an organic compound containing amine (—NH₂) and carboxyl (—COOH) functional groups, along with a side chain specific to each amino acid. The names of amino acids are also represented as standard single letter or three-letter codes in the present disclosure, which are summarized as follows.

| Names | Three-letter Code | Single-letter Code |
|---|---|---|
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

A “conservative substitution” with reference to amino acid sequence refers to replacing an amino acid residue with a different amino acid residue having a side chain with similar physiochemical properties. For example, conservative substitutions can be made among amino acid residues with hydrophobic side chains (e.g., Met, Ala, Val, Leu, and Ile), among amino acid residues with neutral hydrophilic side chains (e.g., Cys, Ser, Thr, Asn and Gln), among amino acid residues with acidic side chains (e.g., Asp, Glu), among amino acid residues with basic side chains (e.g., His, Lys, and Arg), or among amino acid residues with aromatic side chains (e.g., Trp, Tyr, and Phe). As known in the art, conservative substitution usually does not cause significant change in the protein conformational structure, and therefore could retain the biological activity of a protein.

The term “homologous” as used herein refers to a nucleic acid sequence (or its complementary strand) or amino acid sequence that has sequence identity of at least 60% (e.g., at least 65%, 70%, 75%, 80%, 85%, 88%, 90%, 91%, 92%, 93%, 94%, 95%, 96%, 97%, 98%, or 99%) to another sequence when optimally aligned.

“Percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum number of identical amino acids (or nucleic acids). In other words, percent (%) sequence identity of an amino acid sequence (or nucleic acid sequence) can be calculated by dividing the number of amino acid residues (or bases) that are identical relative to the reference sequence to which it is being compared by the total number of the amino acid residues (or bases) in the candidate sequence or in the reference sequence, whichever is shorter. Conservative substitution of the amino acid residues may or may not be considered as identical residues. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al., J. Mol. Biol., 215:403-410 (1990); Stephen F. et al., Nucleic Acids Res., 25:3389-3402 (1997)), ClustalW2 (available on the website of European Bioinformatics Institute, see also, Higgins D. G. et al., Methods in Enzymology, 266:383-402 (1996); Larkin M. A. et al., Bioinformatics (Oxford, England), 23 (21): 2947-8 (2007)), and ALIGN or Megalign (DNASTAR) software. A person skilled in the art may use the default parameters provided by the tool, or may customize the parameters as appropriate for the alignment, such as for example, by selecting a suitable algorithm.

“Effector functions” as used herein refer to biological activities attributable to the binding of Fc region of an antibody to its effectors such as C1 complex and Fc receptor. Exemplary effector functions include: complement dependent cytotoxicity (CDC) mediated by interaction of antibodies and C1q on the C1 complex; antibody-dependent cell-mediated cytotoxicity (ADCC) mediated by binding of Fc region of an antibody to Fc receptor on an effector cell; and phagocytosis. Effector functions can be evaluated using various assays such as Fc receptor binding assay, C1q binding assay, and cell lysis assay.

“Antibody-dependent cellular cytotoxicity” or “ADCC” as used herein refers to a cell-mediated reaction in which effector cells that express Fc receptors (FcRs) recognize bound antibody or antigen-binding fragment on a target cell and subsequently cause lysis of the target cell. “ADCC activity” or “ADCC effect” refers to the ability of the antibody or antigen-binding fragment which is bound on the target cell to elicit an ADCC reaction as described above.

“Complement dependent cytotoxicity” or “CDC” as used herein refers to a mechanism by which antibodies can mediate specific target cell lysis through activation of an organism's complement system. In CDC, the C1q binds the antibody and this binding triggers the complement cascade which leads to the formation of the membrane attack complex (MAC) (C5b to C9) at the surface of the target cell, as a result of the classical pathway complement activation. “CDC activity” or “CDC effect” refers to the ability of the antibody or antigen-binding fragment which is bound on the target cell to elicit a CDC reaction as described above.

“Antibody dependent cellular phagocytosis” or “ADCP” as used herein refers to an immunological mechanism of elimination whereby tumor cells are targeted with monoclonal antibodies to promote their clearance from the body by phagocytic immune cells. The ADCP effect of an antibody can be measured by well-known methods in the art, for example, the methods described in Example 4 of the present disclosure.

“Target cells” as used herein refer to cells to which antibodies comprising an Fc region specifically bind, generally via the protein part that is C-terminal to the Fc region. “Effector cells” are leukocytes which express one or more Fc receptors and perform effector functions. Examples of human leukocytes which mediate ADCC include peripheral blood mononuclear cells (PBMCs), natural killer (NK) cells, monocytes, cytotoxic T cells and neutrophils; with PBMCs and NK cells being preferred. The effector cells may be isolated from a native source thereof, e.g., from blood or PBMCs as is known in the art.

An “isolated” substance has been altered by the hand of man from the natural state. If an “isolated” composition or substance occurs in nature, it has been changed or removed from its original environment, or both. For example, a polynucleotide or a polypeptide naturally present in a living animal is not “isolated”, but the same polynucleotide or polypeptide is “isolated” if it has been sufficiently separated from the coexisting materials of its natural state so as to exist in a substantially pure state. An “isolated nucleic acid sequence” refers to the sequence of an isolated nucleic acid molecule. In certain embodiments, an “isolated antibody or an antigen-binding fragment thereof” refers to the antibody or antigen-binding fragments thereof having a purity of at least 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% as determined by electrophoretic methods (such as SDS-PAGE, isoelectric focusing, capillary electrophoresis), or chromatographic methods (such as ion exchange chromatography or reverse phase HPLC).

The term “vector” as used herein refers to a vehicle into which a polynucleotide encoding a protein may be operably inserted so as to bring about the expression of that protein. A vector may be used to transform, transduce, or transfect a host cell so as to bring about expression of the genetic element it carries within the host cell. Examples of vectors include plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Categories of animal viruses used as vectors include retrovirus (including lentivirus), adenovirus, adeno-associated virus, herpesvirus (e.g., herpes simplex virus), poxvirus, baculovirus, papillomavirus, and papovavirus (e.g., SV40). A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating. A vector can be an expression vector or a cloning vector. The present disclosure provides vectors (e.g., expression vectors) containing the nucleic acid sequence provided herein encoding the antibody or antigen-binding fragment thereof, at least one promoter (e.g., SV40, CMV, EF-1a) operably linked to the nucleic acid sequence, and at least one selection marker. Examples of vectors include, but are not limited to, retrovirus (including lentivirus), adenovirus, adeno-associated virus, herpesvirus (e.g., herpes simplex virus), poxvirus, baculovirus, papillomavirus, papovavirus (e.g., SV40), lambda phage, and M13 phage, plasmid pcDNA3.3, pMD18-T, pOptivec, pCMV, pEGFP, pIRES, pQD-Hyg-GSeu, pALTER, pBAD, pcDNA, pCal, pL, pET, pGEMEX, pGEX, pCI, pEGFT, pSV2, pFUSE, pVITRO, pVIVO, pMAL, pMONO, pSELECT, pUNO, pDUO, Psg5L, pBABE, pWPXL, pBI, p15TV-L, pPro18, pTD, pRS10, pLexA, pACT2.2, pCMV-SCRIPT®, pCDM8, pCDNA1.1/amp, pcDNA3.1, pRc/RSV, PCR 2.1, pEF-1, pFB, pSG5, pXT1, pCDEF3, pSVSPORT, pEF-Bos etc.

The phrase “host cell” as used herein refers to a cell into which an exogenous polynucleotide and/or a vector can be or has been introduced.

The term “subject” includes human and non-human animals. Non-human animals include all vertebrates, e.g., mammals and non-mammals, such as non-human primates, mice, rats, cats, rabbits, sheep, dogs, cows, chickens, amphibians, and reptiles. Except when noted, the terms “patient”, “subject” or “individual” are used herein interchangeably.

The term “anti-tumor activity” means a reduction in tumor cell proliferation, viability, or metastatic activity. For example, anti-tumor activity can be shown by a decline in growth rate of abnormal cells that arises during therapy or tumor size stability or reduction, or longer survival due to therapy as compared to control without therapy. Such activity can be assessed using accepted in vitro or in vivo tumor models, including but not limited to xenograft models, allograft models, mouse mammary tumor virus (MMTV) models, and other known models known in the art to investigate anti-tumor activity.

“Treating” or “treatment” of a disease, disorder or condition as used herein includes preventing or alleviating a disease, disorder or condition, slowing the onset or rate of development of a disease, disorder or condition, reducing the risk of developing a disease, disorder or condition, preventing or delaying the development of symptoms associated with a disease, disorder or condition, reducing or ending symptoms associated with a disease, disorder or condition, generating a complete or partial regression of a disease, disorder or condition, curing a disease, disorder or condition, or some combination thereof.

The term “diagnosis”, “diagnose” or “diagnosing” refers to the identification of a pathological state, disease or condition, such as identification of a LILRB4 related disease, or refer to identification of a subject with a LILRB4 related disease who may benefit from a particular treatment regimen. In some embodiments, diagnosis contains the identification of abnormal amount or activity of LILRB4. In some embodiments, diagnosis refers to the identification of a cancer in a subject.

As used herein, the term “biological sample” or “sample” refers to a biological composition that is obtained or derived from a subject of interest that contains a cellular and/or other molecular entity that is to be characterized and/or identified, for example based on physical, biochemical, chemical and/or physiological characteristics. A biological sample includes, but is not limited to, cells, tissues, organs and/or biological fluids of a subject, obtained by any method known by those of skill in the art. In some embodiments, the biological sample is a fluid sample. In some embodiments, the fluid sample is whole blood, plasma, blood serum, mucus (including nasal drainage and phlegm), peritoneal fluid, pleural fluid, chest fluid, saliva, urine, synovial fluid, cerebrospinal fluid (CSF), thoracentesis fluid, abdominal fluid, ascites or pericardial fluid. In some embodiments, the biological sample is a tissue or cell obtained from stomach, heart, liver, spleen, lung, kidney, skin or blood vessels of the subject.

The term “LILRB4” as used herein, refers to the leukocyte immunoglobulin-like receptor subfamily B member 4, includes any variants, conformations, isoforms and species homologs of LILRB4 which are naturally expressed by cells or are expressed by cells transfected with the LILRB4 gene. For example, LILRB4 described herein may refer to the leukocyte immunoglobulin-like receptor subfamily B member 4 protein derived from any vertebrate source, including mammals such as primates (e.g., humans, monkeys) and rodents (e.g., mice and rats). Exemplary sequence of human LILRB4 protein is, for example as described in UniProtKB Entry No.: Q8NHJ6 or GenBank Accession No. AAB68665.1. The term “LILRB4” as used herein is intended to encompass any form of LILRB4, for example, 1) native unprocessed LILRB4 molecule, “full-length” LILRB4 chain or naturally occurring variants of LILRB4, including, for example, splice variants or allelic variants; 2) any form of LILRB4 that results from processing in the cell; or 3) full length, a fragment (e.g., a truncated form, an extracellular/transmembrane domain) or a modified form (e.g., a mutated form, a glycosylated/PEGylated, a His-tag/immunofluorescence fused form) of LILRB4 subunit generated through recombinant method.

The term “anti-LILRB4 antibody” refers to an antibody that specifically binds to LILRB4 (e.g., human LILRB4). The term “anti-human LILRB4 antibody” or “anti-hLILRB4 antibody” refers to an antibody that specifically binds to human LILRB4.

A “LILRB4 related” or “LILRB4-related” disease, disorder or condition as used herein refers to any disease, disorder or condition caused by, exacerbated by, or otherwise linked to increased or decreased expression or activities of LILRB4. In some embodiments, the LILRB4-related disease, disorder or condition is a disorder related to excessive cell proliferation, such as, for example, cancer. In certain embodiments, the LILRB4-related disease or condition is characterized in expressing or over-expressing of LILRB4 and/or LILRB4 related genes.

The term “pharmaceutically acceptable” indicates that the designated carrier, vehicle, diluent, excipient(s), and/or salt is generally chemically and/or physically compatible with the other ingredients comprising the formulation, and physiologically compatible with the recipient thereof.

The term “LILRB4-expressing cell” as used herein refer to a cell that expresses LILRB4 on the surface of the cell.

### Anti-LILRB4 Antibodies

The present disclosure provides anti-LILRB4 antibodies and antigen-binding fragments thereof. The anti-LILRB4 antibodies and antigen-binding fragments provided herein are capable of binding (e.g., specifically binding) to LILRB4 (e.g., human LILRB4).

Binding affinity of the antibody or antigen-binding fragment thereof provided herein can be represented by K_{D} value, which represents the ratio of dissociation rate to association rate (k_{off}/kₒₙ) when the binding between the antigen and antigen-binding molecule reaches equilibrium. The antigen-binding affinity (e.g., K_{D}) can be appropriately determined using suitable methods known in the art, including, for example, flow cytometry assay. In some embodiments, binding of the antibody or antigen-binding fragment thereof to the antigen at different concentrations can be determined by flow cytometry, the determined mean fluorescence intensity (MFI) can be firstly plotted against antibody concentration, K_{D} value can then be calculated by fitting the dependence of specific binding fluorescence intensity (Y) and the concentration of antibodies (X) into the one site saturation equation: Y=Bₘₐₓ*X/(K_{D}+X) using Prism version 5 (GraphPad Software, San Diego, CA), wherein Bₘₐₓ refers to the maximum specific binding of the tested antibody to the antigen.

Binding of the antibodies or the antigen-binding fragments thereof provided herein to LILRB4 can also be represented by “half maximal effective concentration” (EC₅₀) value, which refers to the concentration of an antibody where 50% of its maximal binding is observed. The EC₅₀ value can be measured by binding assays known in the art, for example, direct or indirect binding assay such as enzyme-linked immunosorbent assay (ELISA), FACS assay, and other binding assays.

In certain embodiments, the antibodies or antigen-binding fragments thereof provided herein are capable of specifically binding to human LILRB4 as measured by FACS assay. For example, the antibodies or antigen-binding fragments thereof provided herein bind to human LILRB4 at an EC₅₀ of no more than 5 nM (e.g., no more than 4 nM, no more than 3 nM, no more than 2 nM, or no more than 1 nM) as measured by FACS assay.

In certain embodiments, the antibodies or antigen-binding fragments thereof provided herein are capable of specifically binding to human LILRB4 and human LILRB3 as measured by FACS assay. In certain embodiments, the antibodies or antigen-binding fragments thereof provided herein bind to human LILRB3 at an EC₅₀ of no more than 15 nM (e.g., no more than 10 nM, no more than 9 nM, no more than 8 nM, no more than 7 nM, no more than 6 nM, no more than 5 nM, no more than 4 nM, no more than 3 nM, no more than 2 nM, or no more than 1 nM) as measured by FACS assay. In certain embodiments, the antibodies or antigen-binding fragments thereof provided herein bind to human LILRB4 at an EC₅₀ of no more than 15 nM (e.g., no more than 10 nM, no more than 9 nM, no more than 8 nM, no more than 7 nM, no more than 6 nM, no more than 5 nM, no more than 4 nM, no more than 3 nM, no more than 2 nM, or no more than 1 nM) as measured by FACS assay.

In certain embodiments, the antibodies or antigen-binding fragments thereof provided herein are capable of specifically binding to human LILRB4 and cynomolgus LILRB4 as measured by FACS assay, which means the antibodies or antigen-binding fragments thereof provided herein are with cross reactivity against human and cynomolgus LILRB4 as measured by FACS assay.

The term “cross reactivity” as used herein refers to the ability of a binding protein to bind a target other than that against which it was raised. Generally, a binding protein will bind its target tissue(s)/antigen(s) with an appropriately high affinity, but will display an appropriately low affinity for non-target normal tissues/antigens. Individual binding proteins are generally selected to meet two criteria: (1) antibody binding, as visualized using staining methods known in the art, to tissue appropriate for the known expression of the antibody target and (2) a similar staining pattern between human and tox species (e.g., mouse and cynomolgus monkey) tissues from the same organ. These and other methods of assessing cross-reactivity are known to one skilled in the art (for example, US20090311253A1).

In certain embodiments, the antibodies or antigen-binding fragments thereof provided herein show potent ADCP effect on a human AML cell line (e.g., as measured by FACS assay). In certain embodiments, the ADCP effect is measured by the method as described in Example 4 of the present disclosure.

In certain embodiments, the antibodies or antigen-binding fragments thereof provided herein show potent ADCC effect on a human AML cell line (e.g., as measured by FACS assay). In certain embodiments, the ADCC effect is measured by the method as described in Example 3 of the present disclosure.

In certain embodiments, the antibodies or antigen-binding fragments thereof provided herein are capable of blocking the LILRB4-fibronectin interaction to reprogram tolerogenic dendritic cells to be mature dendritic cells which stimulate T cell activation as measured by FcγR stimulation assay. In certain embodiments, the LILRB4-fibronectin interaction is measured by the method as described in Example 5 of the present disclosure. In certain embodiments, the antibodies or antigen-binding fragments thereof provided herein are capable of inducing TNF-α production. TNF-α secretion from tolerogenic dendritic cells was used as a readout for evaluating FcγR stimulation.

In certain embodiments, the antibodies or antigen-binding fragments thereof provided herein are capable of reprograming tolerogenic dendritic cells (DCs) to activate T cells. DCs are the most professional antigen presenting cells and play a critical role in orchestrating the immune response between innate and adaptive immunity. Mature DCs show a strong co-stimulatory and T cell activating capacity. Mature DCs have high expression levels of the antigen presentation molecules (HLA-DR and HLA-ABC) and co-stimulatory molecules (CD80 and CD86), to activate T cells for antitumor. The interaction of HLA and T cell receptor induces primary signaling and CD80/CD86 interaction with CD28 on naive T cells triggers co-stimulatory signaling. Both interactions lead to T cell activation and proliferation for defending tumor cells. However, the antigen presentation molecules and co-stimulatory molecules expression on tolerogenic DCs are attenuated while the LILRB4 expression on tolerogenic DCs is upregulated. However, treatment with anti-LILRB4 antibodies (e.g., the anti-LILRB4 antibodies of the present disclosure) on tolerogenic DCs can increase the expression of antigen presentation molecules and co-stimulatory molecules, such as HLA-DR, HLA-ABC and CD86 by blocking LILRB4 signaling. In certain embodiments, the ability of an anti-LILRB4 antibody reprogramming the tolerogenic DCs to activate T cells is measured by the method as described in Example 6 of the present disclosure.

In certain embodiments, the antibodies or antigen-binding fragments thereof provided herein are capable of reversing macrophage-mediated T cell suppression. In certain embodiments, the ability of an anti-LILRB4 antibody reversing macrophage-mediated T cell suppression is measured by the method as described in Example 9.4 of the present disclosure.

In certain embodiments, the antibodies or antigen-binding fragments thereof provided herein are capable of reversing THP-1-mediated T cell suppression. In certain embodiments, the ability of an anti-LILRB4 antibody reversing THP-1-mediated T cell suppression is measured by the method as described in Example 7 of the present disclosure.

In certain embodiments, the antibodies or antigen-binding fragments thereof provided herein are capable of potentiating CD8⁺ T cell-mediated cytotoxicity on THP-1 cells. In certain embodiments, the ability of an anti-LILRB4 antibody potentiating CD8⁺ T cell-mediated cytotoxicity on THP-1 cells is measured by the method as described in Example 9.5 of the present disclosure.

### Illustrative Anti-LILRB4 Antibodies

In certain embodiments, the present disclosure provides antibodies or antigen-binding fragments thereof which bind to LILRB4, comprising:
one, two or three heavy chain complementarity determining regions (HCDR1, HCDR2 and/or HCDR3) contained within any one of the heavy chain variable (VH) region sequences selected from the group consisting of SEQ ID NOs: 23, 31, 39, 47, 55, 63, 71, 79, 87, 95, 103, 111, 119, 127, 135, 143, 151, 154 and 156; and/or
one, two or three light chain complementarity determining regions (LCDR1, LCDR2 and LCDR3) contained within any one of the light chain variable (VL) region sequences selected from the group consisting of SEQ ID NOs: 24, 32, 40, 48, 56, 64, 72, 80, 88, 96, 104, 112, 120, 128, 136, 144, 152, 155 and 157.

A person skilled in the art can define or identify CDR boundaries of a VH or VL region by well-known methods in the art as long as the amino acid sequence of the VH or VL region is known. For example, CDR boundaries for an antibody or antigen-binding fragment thereof may be defined or identified by the conventions of Kabat, IMGT, Chothia, or Al-Lazikani (Al-Lazikani, B., Chothia, C., Lesk, A. M., J. Mol. Biol., 273 (4), 927 (1997); Chothia, C. et al., J Mol Biol. December 5; 186(3):651-63 (1985); Chothia, C. and Lesk, A. M., J. Mol. Biol., 196,901 (1987); Chothia, C. et al., Nature. December 21-28; 342(6252): 877-83 (1989); Kabat E. A. et al., Sequences of Proteins of immunological Interest, 5th Ed. Public Health Service, National Institutes of Health, Bethesda, Md. (1991); Marie-Paule Lefranc et al., Developmental and Comparative Immunology, 27:55-77 (2003); Marie-Paule Lefranc et al., Immunome Research, 1(3), (2005); Marie-Paule Lefranc, Molecular Biology of B cells (second edition), chapter 26, 481-514, (2015)). In some embodiments, the CDR boundaries of the antibodies or antigen-binding fragments thereof provided herein are identified by the convention of Kabat. In some embodiments, the CDR boundaries of the antibodies or antigen-binding fragments thereof provided herein are identified by the convention of IMGT. In some embodiments, the CDR boundaries of the antibodies or antigen-binding fragments thereof provided herein are identified by the convention of Chothia. In some embodiments, the CDR boundaries of the antibodies or antigen-binding fragments thereof provided herein are identified by the convention of Al-Lazikani.

In certain embodiments, the present disclosure provides antibodies or antigen-binding fragments thereof which bind to LILRB4 comprising one or more (e.g., 1, 2, 3, 4, 5, or 6) CDR sequences of an anti-LILRB4 antibody 27-F11-E10-G10, 43-D12-F3-G11, 36-F4-H1-D9, 8-B3-F6-H8, 44-F10-B6-D4, 27-G5-E9-B8, 48-E11-H7-B5, 42-C8-A12-F3-D11, 10-H9-E3-G3, 7-B4-C1-C9, 25-E7-A10-H6, 25-G9-G4-C12, 2-H1-D7-E5-D5, 27-D11-C2-A10, 26-H9-B9-B7, 4-E6-E4-B12, or 2-H8-C9-F7-E2.

Antibody “27-F11-E10-G10” as used herein refers to a mouse monoclonal antibody comprising a heavy chain variable region having the sequence of SEQ ID NO: 23, and a light chain variable region having the sequence of SEQ ID NO: 24.

Antibody “43-D12-F3-G11” as used herein refers to a mouse monoclonal antibody comprising a heavy chain variable region having the sequence of SEQ ID NO: 31, and a light chain variable region having the sequence of SEQ ID NO: 32.

Antibody “36-F4-H1-D9” as used herein refers to a mouse monoclonal antibody comprising a heavy chain variable region having the sequence of SEQ ID NO: 39, and a light chain variable region having the sequence of SEQ ID NO: 40.

Antibody “8-B3-F6-H8” as used herein refers to a mouse monoclonal antibody comprising a heavy chain variable region having the sequence of SEQ ID NO: 47, and a light chain variable region having the sequence of SEQ ID NO: 48.

Antibody “44-F10-B6-D4” as used herein refers to a mouse monoclonal antibody comprising a heavy chain variable region having the sequence of SEQ ID NO: 55, and a light chain variable region having the sequence of SEQ ID NO: 56.

Antibody “27-G5-E9-B8” as used herein refers to a mouse monoclonal antibody comprising a heavy chain variable region having the sequence of SEQ ID NO: 63, and a light chain variable region having the sequence of SEQ ID NO: 64.

Antibody “48-E11-H7-B5” as used herein refers to a mouse monoclonal antibody comprising a heavy chain variable region having the sequence of SEQ ID NO: 71, and a light chain variable region having the sequence of SEQ ID NO: 72.

Antibody “42-C8-A12-F3-D11” as used herein refers to a mouse monoclonal antibody comprising a heavy chain variable region having the sequence of SEQ ID NO: 79, and a light chain variable region having the sequence of SEQ ID NO: 80.

Antibody “10-H9-E3-G3” as used herein refers to a mouse monoclonal antibody comprising a heavy chain variable region having the sequence of SEQ ID NO: 87, and a light chain variable region having the sequence of SEQ ID NO: 88.

Antibody “7-B4-C1-C9” as used herein refers to a mouse monoclonal antibody comprising a heavy chain variable region having the sequence of SEQ ID NO: 95, and a light chain variable region having the sequence of SEQ ID NO: 96.

Antibody “25-E7-A10-H6” as used herein refers to a mouse monoclonal antibody comprising a heavy chain variable region having the sequence of SEQ ID NO: 103, and a light chain variable region having the sequence of SEQ ID NO: 104.

Antibody “25-G9-G4-C12” as used herein refers to a mouse monoclonal antibody comprising a heavy chain variable region having the sequence of SEQ ID NO: 111, and a light chain variable region having the sequence of SEQ ID NO: 112.

Antibody “2-H1-D7-E5-D5” as used herein refers to a mouse monoclonal antibody comprising a heavy chain variable region having the sequence of SEQ ID NO: 119, and a light chain variable region having the sequence of SEQ ID NO: 120.

Antibody “27-D11-C2-A10” as used herein refers to a mouse monoclonal antibody comprising a heavy chain variable region having the sequence of SEQ ID NO: 127, and a light chain variable region having the sequence of SEQ ID NO: 128.

Antibody “26-H9-B9-B7” as used herein refers to a mouse monoclonal antibody comprising a heavy chain variable region having the sequence of SEQ ID NO: 135, and a light chain variable region having the sequence of SEQ ID NO: 136.

Antibody “4-E6-E4-B12” as used herein refers to a mouse monoclonal antibody comprising a heavy chain variable region having the sequence of SEQ ID NO: 143, and a light chain variable region having the sequence of SEQ ID NO: 144.

Antibody “2-H8-C9-F7-E2” as used herein refers to a mouse monoclonal antibody comprising a heavy chain variable region having the sequence of SEQ ID NO: 151, and a light chain variable region having the sequence of SEQ ID NO: 152.

The specific amino acid sequences of the heavy chain variable region and light chain variable region of each exemplary antibody as described above are shown in Table 3 and Table 21 below.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 23, and three light chain CDRs (LCDR1, LCDR2 and LCDR3) contained within the VL region sequence as set forth in SEQ ID NO: 24.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 31, and three light chain CDRs (LCDR1, LCDR2 and LCDR3) contained within the VL region sequence as set forth in SEQ ID NO: 32.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 39, and three light chain CDRs (LCDR1, LCDR2 and LCDR3) contained within the VL region sequence as set forth in SEQ ID NO: 40.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 47, and three light chain CDRs (LCDR1, LCDR2 and LCDR3) contained within the VL region sequence as set forth in SEQ ID NO: 48.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 55, and three light chain CDRs (LCDR1, LCDR2 and LCDR3) contained within the VL region sequence as set forth in SEQ ID NO: 56.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 63, and three light chain CDRs (LCDR1, LCDR2 and LCDR3) contained within the VL region sequence as set forth in SEQ ID NO: 64.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 71, and three light chain CDRs (LCDR1, LCDR2 and LCDR3) contained within the VL region sequence as set forth in SEQ ID NO: 72.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 79, and three light chain CDRs (LCDR1, LCDR2 and LCDR3) contained within the VL region sequence as set forth in SEQ ID NO: 80.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 87, and three light chain CDRs (LCDR1, LCDR2 and LCDR3) contained within the VL region sequence as set forth in SEQ ID NO: 88.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 95, and three light chain CDRs (LCDR1, LCDR2 and LCDR3) contained within the VL region sequence as set forth in SEQ ID NO: 96.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 103, and three light chain CDRs (LCDR1, LCDR2 and LCDR3) contained within the VL region sequence as set forth in SEQ ID NO: 104.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 111, and three light chain CDRs (LCDR1, LCDR2 and LCDR3) contained within the VL region sequence as set forth in SEQ ID NO: 112.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 119, and three light chain CDRs (LCDR1, LCDR2 and LCDR3) contained within the VL region sequence as set forth in SEQ ID NO: 120.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 127, and three light chain CDRs (LCDR1, LCDR2 and LCDR3) contained within the VL region sequence as set forth in SEQ ID NO: 128.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 135, and three light chain CDRs (LCDR1, LCDR2 and LCDR3) contained within the VL region sequence as set forth in SEQ ID NO: 136.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 143, and three light chain CDRs (LCDR1, LCDR2 and LCDR3) contained within the VL region sequence as set forth in SEQ ID NO: 144.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 151, and three light chain CDRs (LCDR1, LCDR2 and LCDR3) contained within the VL region sequence as set forth in SEQ ID NO: 152.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 154, and three light chain CDRs (LCDR1, LCDR2 and LCDR3) contained within the VL region sequence as set forth in SEQ ID NO: 155.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 156, and three light chain CDRs (LCDR1, LCDR2 and LCDR3) contained within the VL region sequence as set forth in SEQ ID NO: 157.

In certain embodiments, the antibodies or antigen-binding fragments thereof provided herein comprise at least one (e.g., 1, 2, or 3) heavy or light chain CDR comprising an amino acid sequence selected from the group consisting of SEQ ID NOs: 17, 18, 19, 20, 21, 22, 25, 26, 27, 28, 29, 30, 33, 34, 35, 36, 37, 38, 41, 42, 43, 44, 45, 46, 49, 50, 51, 52, 53, 54, 57, 58, 59, 60, 61, 62, 65, 66, 67, 68, 69, 70, 73, 74, 75, 76, 77, 78, 81, 82, 83, 84, 85, 86, 89, 90, 91, 92, 93, 94, 97, 98, 99, 100, 101, 102, 105, 106, 107, 108, 109, 110, 113, 114, 115, 116, 117, 118, 121, 122, 123, 124, 125, 126, 129, 130, 131, 132, 133, 134, 137, 138, 139, 140, 141, 142, 145, 146, 147, 148, 149, 150 and 158.

In certain embodiments, the antibodies or antigen-binding fragments thereof provided herein comprise one or two or three of HCDR1, HCDR2 and HCDR3 comprising an amino acid sequence selected from the group consisting of SEQ ID NOs: 17, 18, 19, 25, 26, 27, 33, 34, 35, 41, 42, 43, 49, 50, 51, 57, 58, 59, 65, 66, 67, 73, 74, 75, 81, 82, 83, 89, 90, 91, 97, 98, 99, 105, 106, 107, 113, 114, 115, 121, 122, 123, 129, 130, 131, 137, 138, 139, 145, 146, 147 and 158.

In certain embodiments, the antibodies or antigen-binding fragments thereof provided herein comprise one or two or three of LCDR1, LCDR2 and LCDR3 comprising an amino acid sequence selected from the group consisting of SEQ ID NOs: 20, 21, 22, 28, 29, 30, 36, 37, 38, 44, 45, 46, 52, 53, 54, 60, 61, 62, 68, 69, 70, 76, 77, 78, 84, 85, 86, 92, 93, 94, 100, 101, 102, 108, 109, 110, 116, 117, 118, 124, 125, 126, 132, 133, 134, 140, 141, 142, 148, 149 and 150.

In certain embodiments, the antibodies or antigen-binding fragments thereof provided herein comprise a HCDR1 comprising an amino acid sequence selected from the group consisting of SEQ ID NOs: 17, 25, 33, 41, 49, 57, 65, 73, 81, 89, 97, 105, 113, 121, 129, 137 and 145; a HCDR2 comprising an amino acid sequence selected from the group consisting of SEQ ID NOs: 18, 26, 34, 42, 50, 58, 66, 74, 82, 90, 98, 106, 114, 122, 130, 138, 146 and 158; and a HCDR3 comprising an amino acid sequence selected from the group consisting of SEQ ID NOs: 19, 27, 35, 43, 51, 59, 67, 75, 83, 91, 99, 107, 115, 123, 131, 139 and 147.

In certain embodiments, the antibodies or antigen-binding fragments thereof provided herein comprise a LCDR1 comprising an amino acid sequence selected from the group consisting of SEQ ID NOs: 20, 28, 36, 44, 52, 60, 68, 76, 84, 92, 100, 108, 116, 124, 132, 140 and 148; a LCDR2 comprising an amino acid sequence selected from the group consisting of SEQ ID NOs: 21, 29, 37, 45, 53, 61, 69, 77, 85, 93, 101, 109, 117, 125, 133, 141 and 149; and a LCDR3 comprising an amino acid sequence selected from the group consisting of SEQ ID NOs: 22, 30, 38, 46, 54, 62, 70, 78, 86, 94, 102, 110, 118, 126, 134, 142 and 150.

In certain embodiments, the antibodies or antigen-binding fragments thereof provided herein comprise:
i. a HCDR1 comprising an amino acid sequence as set forth in SEQ ID NO: 17, a HCDR2 comprising an amino acid sequence as set forth in SEQ ID NO: 18, and a HCDR3 comprising an amino acid sequence as set forth in SEQ ID NO: 19;
ii. a HCDR1 comprising an amino acid sequence as set forth in SEQ ID NO: 25, a HCDR2 comprising an amino acid sequence as set forth in SEQ ID NO: 26, and a HCDR3 comprising an amino acid sequence as set forth in SEQ ID NO: 27;
iii. a HCDR1 comprising an amino acid sequence as set forth in SEQ ID NO: 33, a HCDR2 comprising an amino acid sequence as set forth in SEQ ID NO: 34, and a HCDR3 comprising an amino acid sequence as set forth in SEQ ID NO: 35;
iv. a HCDR1 comprising an amino acid sequence as set forth in SEQ ID NO: 41, a HCDR2 comprising an amino acid sequence as set forth in SEQ ID NO: 42, and a HCDR3 comprising an amino acid sequence as set forth in SEQ ID NO: 43;
v. a HCDR1 comprising an amino acid sequence as set forth in SEQ ID NO: 41, a HCDR2 comprising an amino acid sequence as set forth in SEQ ID NO: 158, and a HCDR3 comprising an amino acid sequence as set forth in SEQ ID NO: 43;
vi. a HCDR1 comprising an amino acid sequence as set forth in SEQ ID NO: 49, a HCDR2 comprising an amino acid sequence as set forth in SEQ ID NO: 50, and a HCDR3 comprising an amino acid sequence as set forth in SEQ ID NO: 51;
vii. a HCDR1 comprising an amino acid sequence as set forth in SEQ ID NO: 57, a HCDR2 comprising an amino acid sequence as set forth in SEQ ID NO: 58, and a HCDR3 comprising an amino acid sequence as set forth in SEQ ID NO: 59;
viii. a HCDR1 comprising an amino acid sequence as set forth in SEQ ID NO: 65, a HCDR2 comprising an amino acid sequence as set forth in SEQ ID NO: 66, and a HCDR3 comprising an amino acid sequence as set forth in SEQ ID NO: 67;
ix. a HCDR1 comprising an amino acid sequence as set forth in SEQ ID NO: 73, a HCDR2 comprising an amino acid sequence as set forth in SEQ ID NO: 74, and a HCDR3 comprising an amino acid sequence as set forth in SEQ ID NO: 75;
x. a HCDR1 comprising an amino acid sequence as set forth in SEQ ID NO: 81, a HCDR2 comprising an amino acid sequence as set forth in SEQ ID NO: 82, and a HCDR3 comprising an amino acid sequence as set forth in SEQ ID NO: 83;
xi. a HCDR1 comprising an amino acid sequence as set forth in SEQ ID NO: 89, a HCDR2 comprising an amino acid sequence as set forth in SEQ ID NO: 90, and a HCDR3 comprising an amino acid sequence as set forth in SEQ ID NO: 91;
xii. a HCDR1 comprising an amino acid sequence as set forth in SEQ ID NO: 97, a HCDR2 comprising an amino acid sequence as set forth in SEQ ID NO: 98, and a HCDR3 comprising an amino acid sequence as set forth in SEQ ID NO: 99;
xiii. a HCDR1 comprising an amino acid sequence as set forth in SEQ ID NO: 105, a HCDR2 comprising an amino acid sequence as set forth in SEQ ID NO: 106, and a HCDR3 comprising an amino acid sequence as set forth in SEQ ID NO: 107;
xiv. a HCDR1 comprising an amino acid sequence as set forth in SEQ ID NO: 113, a HCDR2 comprising an amino acid sequence as set forth in SEQ ID NO: 114, and a HCDR3 comprising an amino acid sequence as set forth in SEQ ID NO: 115;
xv. a HCDR1 comprising an amino acid sequence as set forth in SEQ ID NO: 121, a HCDR2 comprising an amino acid sequence as set forth in SEQ ID NO: 122, and a HCDR3 comprising an amino acid sequence as set forth in SEQ ID NO: 123;
xvi. a HCDR1 comprising an amino acid sequence as set forth in SEQ ID NO: 129, a HCDR2 comprising an amino acid sequence as set forth in SEQ ID NO: 130, and a HCDR3 comprising an amino acid sequence as set forth in SEQ ID NO: 131;
xvii. a HCDR1 comprising an amino acid sequence as set forth in SEQ ID NO: 137, a HCDR2 comprising an amino acid sequence as set forth in SEQ ID NO: 138, and a HCDR3 comprising an amino acid sequence as set forth in SEQ ID NO: 139; or
xviii. a HCDR1 comprising an amino acid sequence as set forth in SEQ ID NO: 145, a HCDR2 comprising an amino acid sequence as set forth in SEQ ID NO: 146, and a HCDR3 comprising an amino acid sequence as set forth in SEQ ID NO: 147.

In certain embodiments, the antibodies or antigen-binding fragments thereof provided herein comprise:
i. a LCDR1 comprising an amino acid sequence as set forth in SEQ ID NO: 20, a LCDR2 comprising an amino acid sequence as set forth in SEQ ID NO: 21, and a LCDR3 comprising an amino acid sequence as set forth in SEQ ID NO: 22;
ii. a LCDR1 comprising an amino acid sequence as set forth in SEQ ID NO: 28, a LCDR2 comprising an amino acid sequence as set forth in SEQ ID NO: 29, and a LCDR3 comprising an amino acid sequence as set forth in SEQ ID NO: 30;
iii. a LCDR1 comprising an amino acid sequence as set forth in SEQ ID NO: 36, a LCDR2 comprising an amino acid sequence as set forth in SEQ ID NO: 37, and a LCDR3 comprising an amino acid sequence as set forth in SEQ ID NO: 38;
iv. a LCDR1 comprising an amino acid sequence as set forth in SEQ ID NO: 44, a LCDR2 comprising an amino acid sequence as set forth in SEQ ID NO: 45, NO: 46;
v. a LCDR1 comprising an amino acid sequence as set forth in SEQ ID NO: 52, a LCDR2 comprising an amino acid sequence as set forth in SEQ ID NO: 53, and a LCDR3 comprising an amino acid sequence as set forth in SEQ ID NO: 54;
vi. a LCDR1 comprising an amino acid sequence as set forth in SEQ ID NO: 60, a LCDR2 comprising an amino acid sequence as set forth in SEQ ID NO: 61, and a LCDR3 comprising an amino acid sequence as set forth in SEQ ID NO: 62;
vii. a LCDR1 comprising an amino acid sequence as set forth in SEQ ID NO: 68, a LCDR2 comprising an amino acid sequence as set forth in SEQ ID NO: 69, and a LCDR3 comprising an amino acid sequence as set forth in SEQ ID NO: 70;
viii. a LCDR1 comprising an amino acid sequence as set forth in SEQ ID NO: 76, a LCDR2 comprising an amino acid sequence as set forth in SEQ ID NO: 77, and a LCDR3 comprising an amino acid sequence as set forth in SEQ ID NO: 78;
ix. a LCDR1 comprising an amino acid sequence as set forth in SEQ ID NO: 84, a LCDR2 comprising an amino acid sequence as set forth in SEQ ID NO: 85, and a LCDR3 comprising an amino acid sequence as set forth in SEQ ID NO: 86;
x. a LCDR1 comprising an amino acid sequence as set forth in SEQ ID NO: 92, a LCDR2 comprising an amino acid sequence as set forth in SEQ ID NO: 93, and a LCDR3 comprising an amino acid sequence as set forth in SEQ ID NO: 94;
xi. a LCDR1 comprising an amino acid sequence as set forth in SEQ ID NO: 100, a LCDR2 comprising an amino acid sequence as set forth in SEQ ID NO: 101, and a LCDR3 comprising an amino acid sequence as set forth in SEQ ID NO: 102;
xii. a LCDR1 comprising an amino acid sequence as set forth in SEQ ID NO: 108, a LCDR2 comprising an amino acid sequence as set forth in SEQ ID NO: 109, and a LCDR3 comprising an amino acid sequence as set forth in SEQ ID NO: 110;
xiii. a LCDR1 comprising an amino acid sequence as set forth in SEQ ID NO: 116, a LCDR2 comprising an amino acid sequence as set forth in SEQ ID NO: 117, and a LCDR3 comprising an amino acid sequence as set forth in SEQ ID NO: 118;
xiv. a LCDR1 comprising an amino acid sequence as set forth in SEQ ID NO: 124, a LCDR2 comprising an amino acid sequence as set forth in SEQ ID NO: 125, and a LCDR3 comprising an amino acid sequence as set forth in SEQ ID NO: 126;
xv. a LCDR1 comprising an amino acid sequence as set forth in SEQ ID NO: 132, a LCDR2 comprising an amino acid sequence as set forth in SEQ ID NO: 133, and a LCDR3 comprising an amino acid sequence as set forth in SEQ ID NO: 134;
xvi. a LCDR1 comprising an amino acid sequence as set forth in SEQ ID NO: 140, a LCDR2 comprising an amino acid sequence as set forth in SEQ ID NO: 141, and a LCDR3 comprising an amino acid sequence as set forth in SEQ ID NO: 142; or
xvii. a LCDR1 comprising an amino acid sequence as set forth in SEQ ID NO: 148, a LCDR2 comprising an amino acid sequence as set forth in SEQ ID NO: 149, and a LCDR3 comprising an amino acid sequence as set forth in SEQ ID NO: 150.

In certain embodiments, the antibodies or antigen-binding fragments thereof provided herein comprise:
i. a HCDR1 comprising an amino acid sequence as set forth in SEQ ID NO: 17, a HCDR2 comprising an amino acid sequence as set forth in SEQ ID NO: 18, a HCDR3 comprising an amino acid sequence as set forth in SEQ ID NO: 19, a LCDR1 comprising an amino acid sequence as set forth in SEQ ID NO: 20, a LCDR2 comprising an amino acid sequence as set forth in SEQ ID NO: 21, and a LCDR3 comprising an amino acid sequence as set forth in SEQ ID NO: 22;
ii. a HCDR1 comprising an amino acid sequence as set forth in SEQ ID NO: 25, a HCDR2 comprising an amino acid sequence as set forth in SEQ ID NO: 26, a HCDR3 comprising an amino acid sequence as set forth in SEQ ID NO: 27, a LCDR1 comprising an amino acid sequence as set forth in SEQ ID NO: 28, a LCDR2 comprising an amino acid sequence as set forth in SEQ ID NO: 29, and a LCDR3 comprising an amino acid sequence as set forth in SEQ ID NO: 30;
iii. a HCDR1 comprising an amino acid sequence as set forth in SEQ ID NO: 33, a HCDR2 comprising an amino acid sequence as set forth in SEQ ID NO: 34, a HCDR3 comprising an amino acid sequence as set forth in SEQ ID NO: 35, a LCDR1 comprising an amino acid sequence as set forth in SEQ ID NO: 36, a LCDR2 comprising an amino acid sequence as set forth in SEQ ID NO: 37, and a LCDR3 comprising an amino acid sequence as set forth in SEQ ID NO: 38;
iv. a HCDR1 comprising an amino acid sequence as set forth in SEQ ID NO: 41, a HCDR2 comprising an amino acid sequence as set forth in SEQ ID NO: 42, a HCDR3 comprising an amino acid sequence as set forth in SEQ ID NO: 43, a LCDR1 comprising an amino acid sequence as set forth in SEQ ID NO: 44, a LCDR2 comprising an amino acid sequence as set forth in SEQ ID NO: 45, NO: 46;
v. a HCDR1 comprising an amino acid sequence as set forth in SEQ ID NO: 41, a HCDR2 comprising an amino acid sequence as set forth in SEQ ID NO: 158, a HCDR3 comprising an amino acid sequence as set forth in SEQ ID NO: 43, a LCDR1 comprising an amino acid sequence as set forth in SEQ ID NO: 44, a LCDR2 comprising an amino acid sequence as set forth in SEQ ID NO: 45, and a LCDR3 comprising an amino acid sequence as set forth in SEQ ID NO: 46;
vi. a HCDR1 comprising an amino acid sequence as set forth in SEQ ID NO: 49, a HCDR2 comprising an amino acid sequence as set forth in SEQ ID NO: 50, a HCDR3 comprising an amino acid sequence as set forth in SEQ ID NO: 51, a LCDR1 comprising an amino acid sequence as set forth in SEQ ID NO: 52, a LCDR2 comprising an amino acid sequence as set forth in SEQ ID NO: 53, and a LCDR3 comprising an amino acid sequence as set forth in SEQ ID NO: 54;
vii. a HCDR1 comprising an amino acid sequence as set forth in SEQ ID NO: 57, a HCDR2 comprising an amino acid sequence as set forth in SEQ ID NO: 58, a HCDR3 comprising an amino acid sequence as set forth in SEQ ID NO: 59, a LCDR1 comprising an amino acid sequence as set forth in SEQ ID NO: 60, a LCDR2 comprising an amino acid sequence as set forth in SEQ ID NO: 61, and a LCDR3 comprising an amino acid sequence as set forth in SEQ ID NO: 62;
viii. a HCDR1 comprising an amino acid sequence as set forth in SEQ ID NO: 65, a HCDR2 comprising an amino acid sequence as set forth in SEQ ID NO: 66, a HCDR3 comprising an amino acid sequence as set forth in SEQ ID NO: 67, a LCDR1 comprising an amino acid sequence as set forth in SEQ ID NO: 68, a LCDR2 comprising an amino acid sequence as set forth in SEQ ID NO: 69, NO: 70;
ix. a HCDR1 comprising an amino acid sequence as set forth in SEQ ID NO: 73, a HCDR2 comprising an amino acid sequence as set forth in SEQ ID NO: 74, a HCDR3 comprising an amino acid sequence as set forth in SEQ ID NO: 75, a LCDR1 comprising an amino acid sequence as set forth in SEQ ID NO: 76, a LCDR2 comprising an amino acid sequence as set forth in SEQ ID NO: 77, and a LCDR3 comprising an amino acid sequence as set forth in SEQ ID NO: 78;
x. a HCDR1 comprising an amino acid sequence as set forth in SEQ ID NO: 81, a HCDR2 comprising an amino acid sequence as set forth in SEQ ID NO: 82, a HCDR3 comprising an amino acid sequence as set forth in SEQ ID NO: 83, a LCDR1 comprising an amino acid sequence as set forth in SEQ ID NO: 84, a LCDR2 comprising an amino acid sequence as set forth in SEQ ID NO: 85, and a LCDR3 comprising an amino acid sequence as set forth in SEQ ID NO: 86;
xi. a HCDR1 comprising an amino acid sequence as set forth in SEQ ID NO: 89, a HCDR2 comprising an amino acid sequence as set forth in SEQ ID NO: 90, a HCDR3 comprising an amino acid sequence as set forth in SEQ ID NO: 91, a LCDR1 comprising an amino acid sequence as set forth in SEQ ID NO: 92, a LCDR2 comprising an amino acid sequence as set forth in SEQ ID NO: 93, and a LCDR3 comprising an amino acid sequence as set forth in SEQ ID NO: 94;
xii. a HCDR1 comprising an amino acid sequence as set forth in SEQ ID NO: 97, a HCDR2 comprising an amino acid sequence as set forth in SEQ ID NO: 98, a HCDR3 comprising an amino acid sequence as set forth in SEQ ID NO: 99, a LCDR1 comprising an amino acid sequence as set forth in SEQ ID NO: 100, a LCDR2 comprising an amino acid sequence as set forth in SEQ ID NO: 101, and a LCDR3 comprising an amino acid sequence as set forth in SEQ ID NO: 102;
xiii. a HCDR1 comprising an amino acid sequence as set forth in SEQ ID NO: 105, a HCDR2 comprising an amino acid sequence as set forth in SEQ ID NO: 106, a HCDR3 comprising an amino acid sequence as set forth in SEQ ID NO: 107, a LCDR1 comprising an amino acid sequence as set forth in SEQ ID NO: 108, a LCDR2 comprising an amino acid sequence as set forth in SEQ ID NO: 109, and a LCDR3 comprising an amino acid sequence as set forth in SEQ ID NO: 110;
xiv. a HCDR1 comprising an amino acid sequence as set forth in SEQ ID NO: 113, a HCDR2 comprising an amino acid sequence as set forth in SEQ ID NO: 114, a HCDR3 comprising an amino acid sequence as set forth in SEQ ID NO: 115, a LCDR1 comprising an amino acid sequence as set forth in SEQ ID NO: 116, a LCDR2 comprising an amino acid sequence as set forth in SEQ ID NO: 117, and a LCDR3 comprising an amino acid sequence as set forth in SEQ ID NO: 118;
xv. a HCDR1 comprising an amino acid sequence as set forth in SEQ ID NO: 121, a HCDR2 comprising an amino acid sequence as set forth in SEQ ID NO: 122, a HCDR3 comprising an amino acid sequence as set forth in SEQ ID NO: 123, a LCDR1 comprising an amino acid sequence as set forth in SEQ ID NO: 124, a LCDR2 comprising an amino acid sequence as set forth in SEQ ID NO: 125, and a LCDR3 comprising an amino acid sequence as set forth in SEQ ID NO: 126;
xvi. a HCDR1 comprising an amino acid sequence as set forth in SEQ ID NO: 129, a HCDR2 comprising an amino acid sequence as set forth in SEQ ID NO: 130, a HCDR3 comprising an amino acid sequence as set forth in SEQ ID NO: 131, a LCDR1 comprising an amino acid sequence as set forth in SEQ ID NO: 132, a LCDR2 comprising an amino acid sequence as set forth in SEQ ID NO: 133, and a LCDR3 comprising an amino acid sequence as set forth in SEQ ID NO: 134;
xvii. a HCDR1 comprising an amino acid sequence as set forth in SEQ ID NO: 137, a HCDR2 comprising an amino acid sequence as set forth in SEQ ID NO: 138, a HCDR3 comprising an amino acid sequence as set forth in SEQ ID NO: 139, a LCDR1 comprising an amino acid sequence as set forth in SEQ ID NO: 140, a LCDR2 comprising an amino acid sequence as set forth in SEQ ID NO: 141, and a LCDR3 comprising an amino acid sequence as set forth in SEQ ID NO: 142; or
xviii. a HCDR1 comprising an amino acid sequence as set forth in SEQ ID NO: 145, a HCDR2 comprising an amino acid sequence as set forth in SEQ ID NO: 146, a HCDR3 comprising an amino acid sequence as set forth in SEQ ID NO: 147, a LCDR1 comprising an amino acid sequence as set forth in SEQ ID NO: 148, a LCDR2 comprising an amino acid sequence as set forth in SEQ ID NO: 149, and a LCDR3 comprising an amino acid sequence as set forth in SEQ ID NO: 150.

The SEQ ID NOs of the heavy chain (denoted as “H”) variable region, light chain (denoted as “L”) variable region, HCDRs and LCDRs of each of the 17 monoclonal antibodies described above are shown in Table 1 below. The amino acid sequences of each CDR of the 17 exemplary monoclonal antibodies are shown in Table 2 below. Unless otherwise indicated, the CDR boundaries as described in Table 2 below were defined or identified by the convention of Kabat. The amino acid sequences of each VH and VL of the 17 exemplary monoclonal antibodies are shown in Table 3 below.

TABLE 1
SEQ ID NOs of VH, VL, HCDRs and LCDRs
of 17 exemplary monoclonal antibodies.

| | | Variable | | | |
|---|---|---|---|---|---|
| | | Region | CDR1 | CDR2 | CDR3 |
| | | (SEQ | (SEQ | (SEQ | (SEQ |
| Antibody | Chain | ID NO) | ID NO) | ID NO) | ID NO) |
| 27-F11- | H | 23 | 17 | 18 | 19 |
| E10-G10 | L | 24 | 20 | 21 | 22 |
| 43-D12- | H | 31 | 25 | 26 | 27 |
| F3-G11 | L | 32 | 28 | 29 | 30 |
| 36-F4- | H | 39 | 33 | 34 | 35 |
| H1-D9 | L | 40 | 36 | 37 | 38 |
| 8-B3- | H | 47 | 41 | 42 | 43 |
| F6-H8 | L | 48 | 44 | 45 | 46 |
| 44-F10- | H | 55 | 49 | 50 | 51 |
| B6-D4 | L | 56 | 52 | 53 | 54 |
| 27-G5- | H | 63 | 57 | 58 | 59 |
| E9-B8 | L | 64 | 60 | 61 | 62 |
| 48-E11- | H | 71 | 65 | 66 | 67 |
| H7-B5 | L | 72 | 68 | 69 | 70 |
| 42-C8-A12- | H | 79 | 73 | 74 | 75 |
| F3-D11 | L | 80 | 76 | 77 | 78 |
| 10-H9- | H | 87 | 81 | 82 | 83 |
| E3-G3 | L | 88 | 84 | 85 | 86 |
| 7-B4- | H | 95 | 89 | 90 | 91 |
| C1-C9 | L | 96 | 92 | 93 | 94 |
| 25-E7- | H | 103 | 97 | 98 | 99 |
| A10-H6 | L | 104 | 100 | 101 | 102 |
| 25-G9- | H | 111 | 105 | 106 | 107 |
| G4-C12 | L | 112 | 108 | 109 | 110 |
| 2-H1-D7- | H | 119 | 113 | 114 | 115 |
| E5-D5 | L | 120 | 116 | 117 | 118 |
| 27-D11- | H | 127 | 121 | 122 | 123 |
| C2-A10 | L | 128 | 124 | 125 | 126 |
| 26-H9- | H | 135 | 129 | 130 | 131 |
| B9-B7 | L | 136 | 132 | 133 | 134 |
| 4-E6- | H | 143 | 137 | 138 | 139 |
| E4-B12 | L | 144 | 140 | 141 | 142 |
| 2-H8-C9- | H | 151 | 145 | 146 | 147 |
| F7-E2 | L | 152 | 148 | 149 | 150 |

TABLE 2
Amino acid sequences of each CDR of
17 exemplary monoclonal antibodies.

| | SEQ ID | Amino Acid |
|---|---|---|
| Description | NO | Sequence |
| HCDR1 | 17 | GFTFSSFG |
| | 25 | GFSLTSYG |
| | 33 | GFDFSRYW |
| | 41 | GFDFSRYW |
| | 49 | GFTFSSYA |
| | 57 | GFTFSSFG |
| | 65 | GFTFSSYA |
| | 73 | GFTFSSHG |
| | 81 | GFTFSSFG |
| | 89 | GYTFTNYW |
| | 97 | GFTFSTFG |
| | 105 | GFTFSSHG |
| | 113 | GFTFSSFG |
| | 121 | GFTFSVFG |
| | 129 | GFTFSSFG |
| | 137 | GYTFTSHW |
| | 145 | GYTFTNYW |
| HCDR2 | 18 | ISSGSSTI |
| | 26 | IWADGST |
| | 34 | INPDSSTI |
| | 42 | INPDSSAI |
| | 50 | ISSGGSYI |
| | 58 | ISSGSSTI |
| | 66 | ITIGDDT |
| | 74 | ISSGGIYT |
| | 82 | ISSGSSAM |
| | 90 | INPSNGRT |
| | 98 | ISSGSSTI |
| | 106 | INSGGYYT |
| | 114 | ISSGSSSI |
| | 122 | ISSGSNTI |
| | 130 | ISSGSSTI |
| | 138 | INPSNGRT |
| | 146 | INPSNGRT |
| HCDR3 | 19 | ARSTGTGAMDY |
| | 27 | AKGHWDGFAY |
| | 35 | ARPYYRYDVGYFDV |
| | 43 | ATSMTMDY |
| | 51 | ARQDWDGGDYYAMDY |
| | 59 | ARSTGTGAMDY |
| | 67 | ARGVNSYGSSYRYFDV |
| | 75 | TRGQVGNWYFDV |
| | 83 | ARSDGKGAMDY |
| | 91 | ARSTYYGYDWYFDV |
| | 99 | ARSTGTGAMDY |
| | 107 | ARGQLGNWYFDV |
| | 115 | ARGTGTGAMDY |
| | 123 | ARSSGTGAMDY |
| | 131 | ARSDGKGAMDY |
| | 139 | ARGNLTTVVPFDY |
| | 147 | ARGSLTTVVPFDY |
| LCDR1 | 20 | QDISNY |
| | 28 | SSITSSY |
| | 36 | QSVSNA |
| | 44 | QDIKSY |
| | 52 | QSVLYSSNQKNF |
| | 60 | QDISNY |
| | 68 | QDVSTA |
| | 76 | SSVSY |
| | 84 | QDISNY |
| | 92 | QDINSY |
| | 100 | QDISKY |
| | 108 | SSVSY |
| | 116 | QDISNY |
| | 124 | QDISNY |
| | 132 | QDISNY |
| | 140 | QDINKF |
| | 148 | QDINNY |
| LCDR2 | 21 | YTS |
| | 29 | STS |
| | 37 | YAS |
| | 45 | YAT |
| | 53 | WAS |
| | 61 | YTS |
| | 69 | WAS |
| | 77 | STS |
| | 85 | YTS |
| | 93 | RAN |
| | 101 | YTS |
| | 109 | STS |
| | 117 | YTS |
| | 125 | YTS |
| | 133 | YTS |
| | 141 | FTS |
| | 149 | YTS |
| LCDR3 | 22 | QQGNTLPWT |
| | 30 | HOWSSYPPT |
| | 38 | QQDYSSPWT |
| | 46 | LQHGESPYT |
| | 54 | HQYLSSLT |
| | 62 | QQGNALPWT |
| | 70 | QQHYSIPFT |
| | 78 | QQRSSYPLT |
| | 86 | QQGNTLPWT |
| | 94 | LQCDEFPHT |
| | 102 | QQGNTLPWT |
| | 110 | QQRSSYPLT |
| | 118 | QQGNTLPWT |
| | 126 | QQGNTLPWT |
| | 134 | QQGNTLPWT |
| | 142 | LQYDLLLT |
| | 150 | LQYDILLT |

TABLE 3
Amino acid sequences of each VH and VL of 17 exemplary
monoclonal antibodies.

| Antibody | VH (SEQ ID NO) | VL (SEQ ID NO) |
|---|---|---|
| 27-F11-E10- | EVQLVESGGGLVQPGGSRKLSCA | DIQMTQTTSSLSASLGDRVTI |
| G10 | ASGFTFSSFGMHWVRQAPEKGLE | SCRASQDISNYLNWYQQKPDG |
| | WVAYISSGSSTIYYADTVKGRFS | TVKLLIYYTSRLHSGVPSRFS |
| | ISRDNPKNTLFLQMTSLRSEDTA | GSGSGTDYSLTISNLEQEDIA |
| | MFYCARSTGTGAMDYWGQGTSVT | TYFCQQGNTLPWTFGGGTKLE |
| | VSS (SEQ ID NO: 23) | LK (SEQ ID NO: 24) |
| 43-D12-F3- | EVQVVESGPGLVAPSQSLSITCT | DIVLTQSPAIMSTSPGEKVTL |
| G11 | VSGFSLTSYGVSWVRQPPGKGLE | TCSASSSITSSYLYWYQQKPG |
| | WLGVIWADGSTNYHSALLSRLSI | SSPKLWIYSTSNLASGVPARF |
| | SKDNSKSQVFLKLNSLQTDDTAT | SGSGSGTSYYLTISSMEAEDA |
| | YYCAKGHWDGFAYWGQGTLVAVS | ASYFCHQWSSYPPTFGGGTKL |
| | A (SEQ ID NO: 31) | ELK (SEQ ID NO: 32) |
| 36-F4-H1-D9 | EVKLLESGGGLVQPGGSLKLSCA | DIVMTQAPKVLLVSAGDRVTI |
| | ASGFDFSRYWMIWVRQAPEKGLE | SCKASQSVSNAVVWYQQKPGQ |
| | WIGEINPDSSTINYTPSLKDKFI | SPKLLIFYASNRYTGVPDRFT |
| | ISRDNAKNTLYLQMSKVRSEDTA | GSGYGTDFTFSISTVQAEDLA |
| | LYYCARPYYRYDVGYFDVWGAGT | VYFCQQDYSSPWTFGGGTKLE |
| | TVTVSS (SEQ ID NO: 39) | IK (SEQ ID NO: 40) |
| 8-B3-F6-H8 | EVKVIESGGGLVQPGGSLKLSCA | DIVMTQSPSSMYASLGERVTI |
| | ASGFDFSRYWMSWVRQAPGKGLE | TCKASQDIKSYLSWYQQKPWK |
| | WIGEINPDSSAINYTPSLKDKFI | SPKTLIYYATSLADGVPSRFS |
| | ISRDNAKNTLYLQMSKVRSEDTA | GSGSGQDYSLTISSLESDDTA |
| | LYYCATSMTMDYWGQGTSVTVSS | TYYCLQHGESPYTFGGGTKLE |
| | (SEQ ID NO: 47) | K (SEQ ID NO: 48) |
| 44-F10-B6- | EVHLVESGGGLVKPGGSLKLSCA | DIVMTQSPSSLAVSAGEKVTM |
| D4 | ASGFTFSSYAMSWVRQTPEKRLE | SCKSSQSVLYSSNQKNFLAWY |
| | WVATISSGGSYIYYPDSVKGRFT | QQKPGQSPKLLIYWASTRESG |
| | ISRDNAKNTLYLQMSSLRSEDTA | VPDRFTGSGSGTDFTLTISSV |
| | MYYCARQDWDGGDYYAMDYWGQG | QAEDLAVYYCHQYLSSLTFGA |
| | TSVTVSS (SEQ ID NO: 55) | GTKLELK (SEQ ID NO: |
| | | 56) |
| 27-G5-E9-B8 | EVQLVESGGGLVQPGGSRKLSCA | DIQMTQTTSSLSASLGDSVTF |
| | ASGFTFSSFGMHWVRQAPEKGLE | SCRASQDISNYLNWYQQKPDG |
| | WVAYISSGSSTIYYADTVKGRFT | TVKLLISYTSRLHSGVPSRFS |
| | ISRDNPKNTLFLQMTSLRSEDTA | GSGSGTDYSLTISNLEQEDIA |
| | MYYCARSTGTGAMDYWGQGTSVT | TYFCQQGNALPWTFGGGTKLE |
| | VSS (SEQ ID NO: 63) | IK (SEQ ID NO: 64) |
| 48-E11-H7- | EVQLVESGGGLVKPGGSLKLSCA | DIVMTQSHKFMSTSVGDRVSI |
| B5 | ASGFTFSSYAMSWVRQTPEKRLE | TCKASQDVSTAVAWYQQKPGQ |
| | WVASITIGDDTYYPDSVKGRFTI | SPKLLIYWASTRHTGVPDRFT |
| | SRDHAKNILYLQVSSLRSEDTAM | GSGSGTDYTLTISSVQAEDLA |
| | YSCARGVNSYGSSYRYFDVWGAG | LYYCQQHYSIPFTFGAGTKLE |
| | TTVTVSS (SEQ ID NO: 71) | K (SEQ ID NO: 72) |
| 42-C8-A12- | EVHLVESGGDLVKPGGSLKLSCA | DIVITQSPAIMSASPGEKVTI |
| F3-D11 | ASGFTFSSHGMSWVRQTPDKRLE | NCSASSSVSYMHWFQQTPGTS |
| | WVATISSGGIYTYHPDSVKGRFS | PKLWIYSTSNLASGVPARFSG |
| | ISRDNAKKTLYLQMSSLKSEDTG | SGSGTFYSLTISRMEAEDAAT |
| | IYYCTRGQVGNWYFDVWGAGTTV | YYCQQRSSYPLTFGAGTKLEL |
| | TVSS (SEQ ID NO: 79) | K (SEQ ID NO: 80) |
| 10-H9-E3-G3 | EVQRVESGGGLVQPGGSRKLSCA | DIQMTQTTSSLSASLGDRVTI |
| | ASGFTFSSFGMHWVRQAPEKGLE | SCRASQDISNYLNWYQQKPDG |
| | WVAYISSGSSAMYYADTVKGRFT | TVKLLVYYTSRLHSGVPSRFS |
| | ISRDNPKNTLFLQMTSLRSEDTA | GSGSGTDYSLTISNLEQEDIA |
| | MYYCARSDGKGAMDYWGQGTSVT | TYFCQQGNTLPWTFGGGTKLE |
| | VSS (SEQ ID NO: 87) | IK (SEQ ID NO: 88) |
| 7-B4-C1-C9 | EVQLQQSGTELVKPGASVKLSCK | DIVLTQSPSSMYASLGERVTI |
| | ASGYTFTNYWMHWVKQRPGQGLE | TCKASQDINSYLSWFQQKPGK |
| | WIGEINPSNGRTNYNEKFKTKAT | SPKTLIYRANRLLDGVPSRFS |
| | LTVDKSSSTAYMQLSSLTSEDSA | GGGSGQDYSLTISSLEHEDMG |
| | VYYCARSTYYGYDWYFDVWGAGT | IYYCLQCDEFPHTFGGGTKLE |
| | TVTVSS (SEQ ID NO: 95) | IK (SEQ ID NO: 96) |
| 25-E7-A10- | EVQLVESGGGLVQPGGSRKLSCA | DIQMTQTTSSLSASLGDRVTI |
| H6 | ASGFTFSTFGMHWVRQAPEKGLE | SCRASQDISKYLNWYQQKPDG |
| | WVAYISSGSSTIYYADTVKGRFT | TVKLLIYYTSRLHSGVPSRFS |
| | ISRDNPKNTLFLQMTSLRSEDTA | GSGSGTDYSLTISNLEQEDIA |
| | MYYCARSTGTGAMDYWGQGTSVT | TYFCQQGNTLPWTFGGGTKLE |
| | VSS (SEQ ID NO: 103) | IK (SEQ ID NO: 104) |
| 25-G9-G4- | EVQLVESGGDLVKPGGSLKLSCA | DIVMTQSPAIMSASPGEKVTI |
| C12 | ASGFTFSSHGMSWVRQTPDKRLE | TCSASSSVSYMHWFQQKPGTS |
| | WVATINSGGYYTYYPDSVKGRFT | PKLWIYSTSNLASGVPARFSG |
| | ISRDNAKNTLYLQMSSLKSEDTA | SGSGTSYSLTISRMEAEDAAT |
| | MYYCARGQLGNWYFDVWGAGTTV | YYCQQRSSYPLTFGAGTKLEL |
| | TVSS (SEQ ID NO: 111) | K (SEQ ID NO: 112) |
| 2-H1-D7-E5- | EVHLVESGGGLVQPGGSRKLSCA | DIQMTQTTSSLSASLGDRVTI |
| D5 | ASGFTFSSFGMHWVRQPPEKGLE | SCRTSQDISNYLNWYQQKPDG |
| | WVAYISSGSSSIYYADTVKGRFT | TVKLLIYYTSKLHSGVPSRFS |
| | ISRDNPKNTLFLQMTSLRSEDTA | GSGSGTDYSLTISNLDQEDIA |
| | MYYCARGTGTGAMDYWGQGTSVT | TYFCQQGNTLPWTFGGGTKLE |
| | VSS (SEQ ID NO: 119) | IK (SEQ ID NO: 120) |
| 27-D11-C2- | EVQLVESGGGLVQPGGSRKLSCA | DIQMTQSTSSLSASLGDRVSI |
| A10 | ASGFTFSVFGVHWVRQAPEKGLE | SCRASQDISNYLNWYQQKPDG |
| | WVAYISSGSNTIHYADTVKGRFT | AVKLLIYYTSRLHSGVPSRFS |
| | ISRDIPKNTLFLQMTSLRSEDTA | GSGSGTDYSLTISNLDQEDIA |
| | MYYCARSSGTGAMDYWGQGTSVT | TYFCQQGNTLPWTFGGGTKLE |
| | VSS (SEQ ID NO: 127) | LK (SEQ ID NO: 128) |
| 26-H9-B9-B7 | EVQRVESGGGLVQPGGSRKLSCA | DIVMTQSTSSLSASLGDRVTI |
| | ASGFTFSSFGMHWVRQAPEKGLE | SCRASQDISNYLNWYQQKPDG |
| | WVAYISSGSSTIYYADTVKGRFT | TVKLLIYYTSRLHSGVPSRFS |
| | ISRDNPKNTLFLQMTSLRSEDTA | GSGSGTDYSLTISNLEQEDIA |
| | MYYCARSDGKGAMDYWGQGTSVT | TYFCQQGNTLPWTFGGGTKLE |
| | VSS (SEQ ID NO: 135) | IK (SEQ ID NO: 136) |
| 4-E6-E4-B12 | EVQLKESGAELVKPGASVKLSCK | DIVMTQSPSSLSASLGGKVTI |
| | ASGYTFTSHWMHWVKQRPGQGLE | TCKASQDINKFIAWYQHKPGK |
| | WIGEINPSNGRTNYNEKFTSKAT | GPRLLIHFTSTLQPGIPSRFS |
| | LTVDQSSSTAYMQLSSLTSEDSA | GSGSGRDYSFSISNLEPEDIA |
| | VYYCARGNLTTVVPFDYWGQGTA | TYFCLQYDLLLTFGGGTKLEI |
| | LTVSS (SEQ ID NO: 143) | K (SEQ ID NO: 144) |
| 2-H8-C9-F7- | EVQLQQSGAELVKPGASVKLSCK | DIQMTQTPSSLSASLGGKVTI |
| E2 | ASGYTFTNYWMHWVKQRPGQGLE | TCKASQDINNYIAWYQHKPGK |
| | WIGEINPSNGRTNYNEKFKSKAT | GPRLLIHYTSTLQPGIPSRFS |
| | LTVDKSSSTAYMQLSSLTSEDSA | GSGSGRDYSFSISNLEPEDIA |
| | VYYCARGSLTTVVPFDYWGQGTT | TYFCLQYDILLTFGGGTKLEK |
| | LTVSS (SEQ ID NO: 151) | (SEQ ID NO: 152) |

Given that each of the 17 exemplary monoclonal antibodies can bind to LILRB4 and that antigen-binding specificity is provided primarily by the CDR1, CDR2 and CDR3 regions, the HCDR1, HCDR2 and HCDR3 sequences and LCDR1, LCDR2 and LCDR3 sequences of each of the 17 exemplary monoclonal antibodies can be “mixed and matched” (i.e., CDRs from different antibodies can be mixed and matched, but each antibody must contain a HCDR1, HCDR2 and HCDR3 and a LCDR1, LCDR2 and LCDR3) to create anti-LILRB4 antibodies or antigen-binding fragments thereof of the present disclosure. LILRB4 binding of such “mixed and matched” antibodies can be tested using the binding assays described above and in the Examples. Preferably, when VH CDR sequences are mixed and matched, the HCDR1, HCDR2 and/or HCDR3 sequence from a particular VH sequence is replaced with a structurally similar CDR sequence(s). Likewise, when VL CDR sequences are mixed and matched, the LCDR1, LCDR2 and/or LCDR3 sequence from a particular VL sequence preferably is replaced with a structurally similar CDR sequence(s). For example, the HCDR1s of antibodies 25-G9-G4-C12 and 2-H1-D7-E5-D5 share some structural similarity and therefore are amenable to mixing and matching. It will be readily apparent to a person skilled in the art that novel VH and VL sequences can be created by substituting one or more VH and/or VL CDR sequences with structurally similar sequences from the CDR sequences disclosed herein for the 17 exemplary monoclonal antibodies.

CDRs are known to be responsible for antigen binding. However, it has been found that not all of the 6 CDRs are indispensable or unchangeable. In other words, it is possible to replace or change or modify one or more CDRs in each of the 17 exemplary monoclonal antibodies, yet substantially retain the specific binding affinity to LILRB4.

In certain embodiments, the anti-LILRB4 antibodies and the antigen-binding fragments provided herein comprise a heavy chain CDR3 sequence of one of the anti-LILRB4 antibodies 27-F11-E10-G10, 43-D12-F3-G11, 36-F4-H1-D9, 8-B3-F6-H8, 44-F10-B6-D4, 27-G5-E9-B8, 48-E11-H7-B5, 42-C8-A12-F3-D11, 10-H9-E3-G3, 7-B4-C1-C9, 25-E7-A10-H6, 25-G9-G4-C12, 2-H1-D7-E5-D5, 27-D11-C2-A10, 26-H9-B9-B7, 4-E6-E4-B12, or 2-H8-C9-F7-E2. In certain embodiments, the anti-LILRB4 antibodies and the antigen-binding fragments thereof provided herein comprise a heavy chain CDR3 sequence selected from the group consisting of SEQ ID NOs: 19, 27, 35, 43, 51, 59, 67, 75, 83, 91, 99, 107, 115, 123, 131, 139, and 147. Heavy chain CDR3 regions are located at the center of the antigen-binding site, and therefore are believed to make the most contact with antigen and provide the most free energy to the affinity of antibody to antigen. It is also believed that the heavy chain CDR3 is by far the most diverse CDR of the antigen-binding site in terms of length, amino acid composition and conformation by multiple diversification mechanisms (Tonegawa S. Nature. 302:575-81). The diversity in the heavy chain CDR3 is sufficient to produce most antibody specificities (Xu J L, Davis M M. Immunity. 13:37-45) as well as desirable antigen-binding affinity (Schier R, et al., J Mol Biol. 263:551-67).

In certain embodiments, the antibodies or antigen-binding fragments thereof provided herein comprise a VH region having an amino acid sequence as set forth in SEQ ID NOs: 23, 31, 39, 47, 55, 63, 71, 79, 87, 95, 103, 111, 119, 127, 135, 143, 151, 154 or 156, or a homologous sequence thereof having at least 80% (e.g., at least 85%, at least 90%, at least 91%, at least 92%, at least 93%, at least 94%, at least 95%, at least 96%, at least 97%, at least 98%, or at least 99%) sequence identity to SEQ ID NOs: 23, 31, 39, 47, 55, 63, 71, 79, 87, 95, 103, 111, 119, 127, 135, 143, 151, 154 or 156.

In certain embodiments, the antibodies or antigen-binding fragments thereof provided herein comprise a VL region having an amino acid sequence as set forth in SEQ ID NOs: 24, 32, 40, 48, 56, 64, 72, 80, 88, 96, 104, 112, 120, 128, 136, 144, 152, 155 or 157, or a homologous sequence thereof having at least 80% (e.g., at least 85%, at least 90%, at least 91%, at least 92%, at least 93%, at least 94%, at least 95%, at least 96%, at least 97%, at least 98%, or at least 99%) sequence identity to SEQ ID NOs: 24, 32, 40, 48, 56, 64, 72, 80, 88, 96, 104, 112, 120, 128, 136, 144, 152, 155 or 157.

In certain embodiments, the antibodies or antigen-binding fragments thereof provided herein comprise a VH/VL amino acid sequence pair selected from the group consisting of SEQ ID NOs: 23/24, 31/32, 39/40, 47/48, 55/56, 63/64, 71/72, 79/80, 87/88, 95/96, 103/104, 111/112, 119/120, 127/128, 135/136, 143/144, 151/152, 154/155, and 156/157.

In certain embodiments, the antibodies and antigen-binding fragments thereof provided herein comprise suitable framework region (FR) sequences, as long as the antibodies and antigen-binding fragments thereof can bind to LILRB4. The CDR sequences provided in Table 2 above are obtained from mouse antibodies, but they can be grafted to any suitable FR sequences of any suitable species such as mouse, human, rat, rabbit, among others, using suitable methods known in the art such as recombinant techniques.

In certain embodiments, the antibodies and antigen-binding fragments thereof provided herein are humanized. A humanized antibody or antigen-binding fragment thereof is desirable in its reduced immunogenicity in human. A humanized antibody is chimeric in its variable regions, as non-human CDR sequences are grafted to human or substantially human FR sequences. Humanization of an antibody or antigen-binding fragment can be essentially performed by substituting the non-human (such as murine) CDR genes for the corresponding human CDR genes in a human immunoglobulin gene (see, for example, Jones et al., (1986) Nature 321:522-525; Riechmann et al., (1988) Nature 332:323-327; Verhoeyen et al., (1988) Science 239:1534-1536).

Suitable human heavy chain and light chain variable domains can be selected to achieve this purpose using methods known in the art. In an illustrative example, “best-fit” approach can be used, where a non-human (e.g., rodent) antibody variable domain sequence is screened or BLASTed against a database of known human variable domain sequences, and the human sequence closest to the non-human query sequence is identified and used as the human scaffold for grafting the non-human CDR sequences (see, for example, Sims et al., (1993) J. Immunol. 151:2296; Chothia et al., (1987) J. Mot. Biol. 196:901). Alternatively, a framework derived from the consensus sequence of all human antibodies may be used for the grafting of the non-human CDRs (see, for example, Carter et al., (1992) Proc. Natl. Acad. Sci. USA, 89:4285; Presta et al., (1993) J. Immunol., 151:2623).

In some embodiments, the anti-LILRB4 antibodies or antigen-binding fragments thereof provided herein are humanized. In certain embodiments, the humanized antibodies or antigen-binding fragments thereof provided herein are composed of substantially all human sequences except for the CDR sequences which are non-human. In some embodiments, the variable region FRs, and constant regions if present, are entirely or substantially from human immunoglobulin sequences. The human FR sequences and human constant region sequences may be derived from different human immunoglobulin genes, for example, FR sequences derived from one human antibody and constant region from another human antibody. In some embodiments, the humanized antibody or antigen-binding fragment thereof comprises human heavy chain HFR1, HFR2, HFR3 and HFR4, and/or light chain LFR1, LFR2, LFR3 and LFR4.

In some embodiments, the present disclosure provides a humanized antibody or antigen-binding fragment thereof of clone 2-H1-D7-E5-D5 (also referred to as “humanized 2-H1-D7-E5-D5” or “h2-H1-D7-E5-D5” in the present disclosure). In some embodiments, the humanized 2-H1-D7-E5-D5 or antigen-binding fragment thereof provided herein comprises one, two or three HCDRs (HCDR1, HCDR2 and/or HCDR3) contained within the heavy chain variable region sequence of SEQ ID NO: 154; and/or comprises one, two or three LCDRs (LCDR1, LCDR2 and/or LCDR3) contained within the light chain variable region sequence of SEQ ID NO: 155. In some embodiments, the humanized 2-H1-D7-E5-D5 or antigen-binding fragment thereof provided herein comprises HCDR1, HCDR2 and HCDR3 contained within the heavy chain variable region sequence of SEQ ID NO: 154; and comprises LCDR1, LCDR2 and LCDR3 contained within the light chain variable region sequence of SEQ ID NO: 155. In some embodiments, the humanized 2-H1-D7-E5-D5 or antigen-binding fragment thereof provided herein comprises a HCDR1 comprising an amino acid sequence as set forth in SEQ ID NO: 113, a HCDR2 comprising an amino acid sequence as set forth in SEQ ID NO: 114, a HCDR3 comprising an amino acid sequence as set forth in SEQ ID NO: 115, a LCDR1 comprising an amino acid sequence as set forth in SEQ ID NO: 116, a LCDR2 comprising an amino acid sequence as set forth in SEQ ID NO: 117, and a LCDR3 comprising an amino acid sequence as set forth in SEQ ID NO: 118. The amino acid sequences of SEQ ID NOs: 154 and 155 are shown in Table 21 below, the amino acid sequences of SEQ IDs: 113-118 are shown in Table 2 above and Table 21 below.

In some embodiments, the present disclosure provides a humanized antibody or antigen-binding fragment thereof of clone 8-B3-F6-H8 (also referred to as “humanized 8-B3-F6-H8” or “h8-B3-F6-H8” in the present disclosure). In some embodiments, the humanized 8-B3-F6-H8 or antigen-binding fragment thereof provided herein comprises one, two or three HCDRs (HCDR1, HCDR2 and/or HCDR3) contained within the heavy chain variable region sequence of SEQ ID NO: 156; and/or comprises one, two or three LCDRs (LCDR1, LCDR2 and/or LCDR3) contained within the light chain variable region sequence of SEQ ID NO: 157. In some embodiments, the humanized 8-B3-F6-H8 or antigen-binding fragment thereof provided herein comprises HCDR1, HCDR2 and HCDR3 contained within the heavy chain variable region sequence of SEQ ID NO: 156; and comprises LCDR1, LCDR2 and LCDR3 contained within the light chain variable region sequence of SEQ ID NO: 157. In some embodiments, the humanized 8-B3-F6-H8 or antigen-binding fragment thereof provided herein comprises a HCDR1 comprising an amino acid sequence as set forth in SEQ ID NO: 41, a HCDR2 comprising an amino acid sequence as set forth in SEQ ID NO: 42, a HCDR3 comprising an amino acid sequence as set forth in SEQ ID NO: 43, a LCDR1 comprising an amino acid sequence as set forth in SEQ ID NO: 44, a LCDR2 comprising an amino acid sequence as set forth in SEQ ID NO: 45, and a LCDR3 comprising an amino acid sequence as set forth in SEQ ID NO: 46. In some embodiments, the humanized 8-B3-F6-H8 or antigen-binding fragment thereof provided herein comprises a HCDR1 comprising an amino acid sequence as set forth in SEQ ID NO: 41, a HCDR2 comprising an amino acid sequence as set forth in SEQ ID NO: 158, a HCDR3 comprising an amino acid sequence as set forth in SEQ ID NO: 43, a LCDR1 comprising an amino acid sequence as set forth in SEQ ID NO: 44, a LCDR2 comprising an amino acid sequence as set forth in SEQ ID NO: 45, and a LCDR3 comprising an amino acid sequence as set forth in SEQ ID NO: 46. The amino acid sequences of SEQ ID NOs: 156 and 157 are shown in Table 21 below, the amino acid sequences of SEQ ID NOs: 41-46 and 158 are shown in Table 2 above and Table 21 below.

TABLE 21
Amino acid sequences of HCDRs, LCDRs, VH regions and
VL regions of humanized antibodies h2-H1-D7-E5-D5 and
h8-B3-F6-H8

| | Sequence | SEQ ID | |
|---|---|---|---|
| Antibody | Description | NO | Amino Acid Sequence |
| h2-H1-D7- | HCDR1 | 113 | GFTFSSFG |
| E5-D5 | | | |
| | HCDR2 | 114 | ISSGSSSI |
| | HCDR3 | 115 | ARGTGTGAMDY |
| | LCDR1 | 116 | QDISNY |
| | LCDR2 | 117 | YTS |
| | LCDR3 | 118 | QQGNTLPWT |
| | VH | 154 | EVQLVESGGGLVQPGGSLRLSCAASGFTFS |
| | | | SFGMHWVRQAPGKGLEWVAYISSGSSSIYY |
| | | | ADTVKGRFTISRDNAKNSLYLQMNSLRAED |
| | | | TAVYYCARGTGTGAMDYWGQGTLVTVSS |
| | VL | 155 | DIQMTQSPSSLSASVGDRVTITCRTSQDIS |
| | | | NYLNWYQQKPGKAPKLLIYYTSKLHSGVPS |
| | | | RFSGSGSGTDFTFTISSLQPEDIATYFCQQ |
| | | | GNTLPWTFGGGTKLEIK |
| h8-B3-F6- | HCDR1 | 41 | GFDFSRYW |
| H8 | | | |
| | HCDR2 | 158 | INPESSAI |
| | HCDR3 | 43 | ATSMTMDY |
| | LCDR1 | 44 | QDIKSY |
| | LCDR2 | 45 | YAT |
| | LCDR3 | 46 | LQHGESPYT |
| | VH | 156 | EVQLVESGGGLVQPGGSLRLSCAASGFDFS |
| | | | RYWMSWVRQAPGKGLVWIGEINPESSAINY |
| | | | APSLKDRFTISRDNAKNTLYLQMNSLRAED |
| | | | TAVYYCATSMTMDYWGQGTLVTVSS |
| | VL | 157 | DIQMTQSPSSLSASVGDRVTITCKASQDIK |
| | | | SYLSWYQQKPGKAPKTLIYYATSLAGGVPS |
| | | | RFSGSGSGTDYTLTISSLQPEDFATYYCLQ |
| | | | HGESPYTFGGGTKLEIK |

In some embodiments, the FR regions derived from human may comprise the same amino acid sequence as the human immunoglobulin from which it is derived. In some embodiments, one or more amino acid residues of the human FR are substituted with the corresponding residues from the parent non-human antibody. This may be desirable in certain embodiments to make the humanized antibody or its fragment closely approximate the non-human parent antibody structure, so as to optimize binding characteristics (for example, increase binding affinity). In certain embodiments, the humanized antibody or antigen-binding fragment thereof provided herein comprises no more than 10, 9, 8, 7, 6, 5, 4, 3, 2, or 1 amino acid residue substitutions in each of the human FR sequences, or no more than 10, 9, 8, 7, 6, 5, 4, 3, 2, or 1 amino acid residue substitutions in all the FR sequences of a heavy or a light chain variable domain. In some embodiments, such change in amino acid residue could be present in heavy chain FR regions only, in light chain FR regions only, or in both chains. In certain embodiments, one or more amino acids of the human FR sequences are randomly mutated to increase binding affinity. In certain embodiments, one or more amino acids of the human FR sequences are back mutated to the corresponding amino acid(s) of the parent non-human antibody so as to increase binding affinity.

In some embodiments, the anti-LILRB4 antibodies and antigen-binding fragments thereof provided herein comprise all or a portion of the heavy chain variable domain and/or all or a portion of the light chain variable domain. In one embodiment, the anti-LILRB4 antibody or antigen-binding fragment thereof provided herein is a single domain antibody which consists of all or a portion of the heavy chain variable domain provided herein. More information of such a single domain antibody is available in the art (see, e.g., U.S. Pat. No. 6,248,516).

In certain embodiments, the anti-LILRB4 antibodies or antigen-binding fragments thereof provided herein further comprise an Fc region. In certain embodiments, the anti-LILRB4 antibodies or antigen-binding fragments thereof provided herein further comprise an Fc region of human immunoglobulin (Ig). In certain embodiments, the anti-LILRB4 antibodies or antigen-binding fragments thereof provided herein further comprise a constant region, which optionally further comprises a heavy chain and/or a light chain constant region. In certain embodiments, the heavy chain constant region comprises CH1, hinge, and/or CH2-CH3 regions (or optionally CH2-CH3-CH4 regions). In certain embodiments, the anti-LILRB4 antibodies or antigen-binding fragments thereof provided herein comprise heavy chain constant regions of human IgG1, IgG2, IgG3, IgG4, IgA1, IgA2 or IgM. In certain embodiments, the anti-LILRB4 antibodies or antigen-binding fragments thereof provided herein comprise a lambda (2) light chain or a kappa (κ) light chain. The constant region of the anti-LILRB4 antibodies or antigen-binding fragments thereof provided herein may be identical to the wild-type constant region sequence or be different in one or more mutations.

In certain embodiments, the heavy chain constant region comprises an Fc region. Fc region is known to mediate effector functions such as antibody-dependent cellular cytotoxicity (ADCC) and complement-dependent cytotoxicity (CDC) of the antibody. Fc regions of different Ig isotypes have different abilities to induce effector functions. For example, Fc regions of IgG1 and IgG3 have been recognized to induce both ADCC and CDC more effectively than those of IgG2 and IgG4. In certain embodiments, the anti-LILRB4 antibodies and antigen-binding fragments thereof provided herein comprises an Fc region of IgG1, or IgG3 isotype, which could induce ADCC or CDC; or alternatively, a constant region of IgG4 or IgG2 isotype, which has reduced or depleted effector function. In some embodiments, the Fc region derived from human IgG1 with enhanced effector functions. In some embodiments, the Fc region comprises an amino acid sequence as set forth in SEQ ID NO: 153.

(SEQ ID NO: 153)
APELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPA
PIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHE
ALHNHYTQKSLSLSPGK

In certain embodiments, the antibodies or antigen-binding fragments thereof provided herein have a specific binding affinity to human LILRB4 which is sufficient to provide for diagnostic and/or therapeutic use.

The antibodies or antigen-binding fragments thereof provided herein can be a monoclonal antibody, a polyclonal antibody, a humanized antibody, a human antibody, a chimeric antibody, a recombinant antibody, a bispecific antibody, a multi-specific antibody, a labeled antibody, a bivalent antibody, an anti-idiotypic antibody, or a fusion protein. A recombinant antibody is an antibody prepared in vitro using recombinant methods rather than in animals.

In certain embodiments, the present disclosure provides an anti-LILRB4 antibody or antigen-binding fragment thereof, which competes for binding to LILRB4 with the antibody or antigen-binding fragment thereof provided herein. In certain embodiments, the present disclosure provides an anti-LILRB4 antibody or antigen-binding fragment thereof, which competes for binding to human LILRB4 with any one of antibodies 27-F11-E10-G10, 43-D12-F3-G11, 36-F4-H1-D9, 8-B3-F6-H8, 44-F10-B6-D4, 27-G5-E9-B8, 48-E11-H7-B5, 42-C8-A12-F3-D11, 10-H9-E3-G3, 7-B4-C1-C9, 25-E7-A10-H6, 25-G9-G4-C12, 2-H1-D7-E5-D5, 27-D11-C2-A10, 26-H9-B9-B7, 4-E6-E4-B12, or 2-H8-C9-F7-E2. In some embodiments, the present disclosure provides an anti-LILRB4 antibody or antigen-binding fragment thereof, which competes for the same epitope with the antibody or antigen-binding fragment thereof provided herein.

The ability to “block binding” or “compete for the same epitope” as used herein refers to the ability of an antibody or antigen-binding fragment to inhibit the binding interaction between two molecules (e.g., human LILRB4 and an anti-LILRB4 antibody) to any detectable degree. In certain embodiments, an antibody or antigen-binding fragment that blocks binding between two molecules inhibits the binding interaction between the two molecules by at least 85%, or at least 90%. In certain embodiments, this inhibition may be greater than 85%, or greater than 90%.

Those skilled in the art will recognize that it is possible to determine, without undue experimentation, if a human monoclonal antibody binds to the same epitope as the antibody of present disclosure (e.g., mouse monoclonal antibodies 27-F11-E10-G10, 43-D12-F3-G11, 36-F4-H1-D9, 8-B3-F6-H8, 44-F10-B6-D4, 27-G5-E9-B8, 48-E11-H7-B5, 42-C8-A12-F3-D11, 10-H9-E3-G3, 7-B4-C1-C9, 25-E7-A10-H6, 25-G9-G4-C12, 2-H1-D7-E5-D5, 27-D11-C2-A10, 26-H9-B9-B7, 4-E6-E4-B12, or 2-H8-C9-F7-E2) by ascertaining whether the former prevents the latter from binding to a LILRB4 antigen polypeptide. If the test antibody competes with the antibody of the present disclosure, as shown by a decrease in binding by the antibody of present disclosure to the LILRB4 antigen polypeptide, then the two antibodies bind to the same, or a closely related, epitope. Or if the binding of a test antibody to the LILRB4 antigen polypeptide was inhibited by the antibody of the present disclosure, then the two antibodies bind to the same, or a closely related, epitope.

In certain embodiments, the present disclosure provides anti-LILRB4 antibodies or antigen-binding fragments thereof which compete for binding to LILRB4 with Antibody IO-202. In certain embodiments, the anti-LILRB4 antibody or antigen-binding fragment which competes for binding to LILRB4 with the antibody or antigen-binding fragment thereof provided herein is not IO-202. The information of IO-202 is described in, for example, WO2016144728A2, WO2018022881A2, Mi Deng et al., Nature. 2018 October; 562 (7728): 605-609, Xun Gui et al., Cancer Immunol Res 2019; 7:1244-57).

In certain embodiments, the present disclosure provides anti-LILRB4 antibodies or antigen-binding fragments thereof which compete for binding to LILRB4 with Antibody NGM831. In certain embodiments, the anti-LILRB4 antibody or antigen-binding fragment which competes for binding to LILRB4 with the antibody or antigen-binding fragment thereof provided herein is not NGM831. The information of NGM831 is described in, for example, US20210221887A1, Kevin J Paavola et al., Cancer Immunol Res. 2021 November; 9(11):1283-1297.

“IO-202” as used herein refers to an antibody or antigen-binding fragment thereof comprising one or two or three heavy chain CDRs contained within a heavy chain variable region having an amino acid sequence of SEQ ID NO: 7, and one or two or three light chain CDRs contained within a light chain variable region having an amino acid sequence of SEQ ID NO: 8. In certain embodiments, IO-202 comprises a HCDR1 comprising an amino acid sequence of SEQ ID NO: 1, a HCDR2 comprising an amino acid sequence of SEQ ID NO: 2, a HCDR3 comprising an amino acid sequence of SEQ ID NO: 3, a LCDR1 comprising an amino acid sequence of SEQ ID NO: 4, a LCDR2 comprising an amino acid sequence of SEQ ID NO: 5, and a LCDR3 comprising an amino acid sequence of SEQ ID NO: 6. The amino acid sequences of each CDR, VH region and VL region of IO-202 are shown in Table 4 below. The CDR boundaries of IO-202 in Table 4 below are identified by the convention of IMGT.

“NGM831” as used herein refers to an antibody or antigen-binding fragment thereof comprising one or two or three heavy chain CDRs contained within a heavy chain variable region having an amino acid sequence of SEQ ID NO: 15, and one or two or three light chain CDRs contained within a light chain variable region having an amino acid sequence of SEQ ID NO: 16. In certain embodiments, NGM831 comprises a HCDR1 comprising an amino acid sequence of SEQ ID NO: 9, a HCDR2 comprising an amino acid sequence of SEQ ID NO: 10, a HCDR3 comprising an amino acid sequence of SEQ ID NO: 11, a LCDR1 comprising an amino acid sequence of SEQ ID NO: 12, a LCDR2 comprising an amino acid sequence of SEQ ID NO: 13, and a LCDR3 comprising an amino acid sequence of SEQ ID NO: 14. The amino acid sequences of each CDR, VH region and VL region of NGM831 are shown in Table 4 below. The CDR boundaries of NGM831 in Table 4 below are identified by the convention of IMGT.

TABLE 4
Amino acid sequences of HCDRs, LCDRs, VH regions and
VL regions of IO-202 and NGM831

| | Sequence | SEQ ID | |
|---|---|---|---|
| Antibody | Description | NO | Amino Acid Sequence |
| IO-202 | HCDR1 | 1 | GIDFSNHYY |
| | HCDR2 | 2 | IFSGDSAST |
| | HCDR3 | 3 | CARGMSTNDWASDL |
| | LCDR1 | 4 | ESINSIY |
| | LCDR2 | 5 | RAS |
| | LCDR3 | 6 | CQQSYDWGDVENT |
| | VH | 7 | EVQLLESGGGLVQPGGSLRLSCAASGIDFSN |
| | | | HYYMYWVRQAPGKGLEWIGSIFSGDSASTYY |
| | | | ADSAKGRFTISRDNSKNTLYLQMNSLRAEDT |
| | | | AVYYCARGMSTNDWASDLWGQGTLVTVSS |
| | VL | 8 | DIQMTQSPSSLSASVGDRVTITCQASESINS |
| | | | IYLAWYQQKPGKAPKLLIYRASTLASGVPSR |
| | | | FSGSGSGTDFTLTISSLQPEDFATYYCQQSY |
| | | | DWGDVENTFGGGTKVEIK |
| NGM831 | HCDR1 | 9 | GYTFTDYN |
| | HCDR2 | 10 | IYPSNGGT |
| | HCDR3 | 11 | CARVPYYDYLYYYAMDY |
| | LCDR1 | 12 | SSVSF |
| | LCDR2 | 13 | ATS |
| | LCDR3 | 14 | CQQWSTNPYMYT |
| | VH | 15 | EVQLQQSGPEVKPGASVKISCKASGYTFTDY |
| | | | NMDWVKQSHGKSEWIGYIYPSNGGTGYNQKF |
| | | | KSKATLTVDKSSNTAYMELHSLTSEDSAVYY |
| | | | CARVPYYDYLYYYAMDYWGQGTSVTVSS |
| | VL | 16 | QIVLSQSPAILSASPGEKVTMACRASSSVSF |
| | | | MHWYQQKPGSSPQPWIYATSNLASGVPARFS |
| | | | GSGSGTSYSLTISRVEAEDAATYYCQQWSTN |
| | | | PYMYTFGGGTKLEIK |

### Antibody Variants

The antibodies and antigen-binding fragments thereof provided herein also encompass various variants of the antibody sequences provided herein.

In certain embodiments, the antibody variants comprise one or more amino acid residue substitutions or modifications yet retains binding affinity to LILRB4. In certain embodiments, at least one of the substitutions or modifications is in one or more of the CDR sequences of the VH region or VL region. In certain embodiments, at least one of the substitutions or modifications is in one or more of the non-CDR sequences of the VH region or VL region. In certain embodiments, the antibodies or antigen-binding fragments thereof provided herein further comprise one or more non-natural amino acid (NNAA) substitution. In certain embodiments, the NNAA is capable of being conjugated.

For example, the antibody variants comprise one or more amino acid residue substitutions or modifications in one or more of the CDR sequences provided in Table 2 or Table 21 above, one or more of the non-CDR sequences of the heavy chain variable region or light chain variable region provided in Table 3 or Table 21 above, and/or the constant region (e.g., Fc region). Such variants retain binding specificity to LILRB4 of their parent antibodies, but have one or more desirable properties conferred by the modification(s) or substitution(s). For example, the antibody variants may have improved antigen-binding affinity, improved glycosylation pattern, reduced risk of glycosylation, reduced deamination, enhanced effector function(s), improved FcRn receptor binding, increased pharmacokinetic half-life, pH sensitivity, and/or compatibility to conjugation (e.g., one or more introduced cysteine residues), etc.

The parent antibody sequence may be screened to identify suitable or preferred residues to be modified or substituted, using methods known in the art, for example, “alanine scanning mutagenesis” (see, for example, Cunningham and Wells (1989) Science, 244:1081-1085). Briefly, target residues (e.g., charged residues such as Arg, Asp, His, Lys, and Glu) can be identified and replaced by a neutral or negatively charged amino acid (e.g., alanine or polyalanine), and the modified antibodies are produced and screened for the interested property. If substitution at a particular amino acid location demonstrates an interested functional change, then the position can be identified as a potential residue for modification or substitution. The potential residues may be further assessed by substituting with a different type of residue (e.g., cysteine residue, positively charged residue, etc.).

### Affinity Variants

Affinity variants of antibodies may contain modifications or substitutions in one or more CDR sequences provided in Table 2 or Table 21 above, one or more FR sequences, or the heavy or light chain variable region sequences provided in Table 3 or Table 21 above. FR sequences can be readily identified by a person skilled in the art based on the CDR sequences in Table 2 or Table 21 above and variable region sequences in Table 3 or Table 21 above, as it is well-known in the art that a CDR region is flanked by two FR regions in the variable region. The affinity variants retain specific binding affinity to LILRB4 of the parent antibody, or even have improved LILRB4 binding affinity over the parent antibody. In certain embodiments, at least one (or all) of the substitution(s) in the CDR sequences, FR sequences, or variable region sequences comprises a conservative substitution.

A person skilled in the art will understand that in the CDR sequences provided in Table 2 or Table 21 above, and variable region sequences provided in Table 3 or Table 21 above, one or more amino acid residues may be substituted yet the resulting antibody or antigen-binding fragment still retain the binding affinity or binding capacity to LILRB4, or even have an improved binding affinity or capacity. Various methods known in the art can be used to achieve this purpose. For example, a library of antibody variants (such as Fab or scFv variants) can be generated and expressed with phage display technology, and then screened for the binding affinity to human LILRB4. For another example, computer software can be used to virtually simulate the binding of the antibodies to human LILRB4, and identify the amino acid residues on the antibodies which form the binding interface. Such residues may be either avoided in the substitution so as to prevent reduction in binding affinity, or targeted for substitution to provide for a stronger binding.

In certain embodiments, the humanized antibody or antigen-binding fragment thereof provided herein comprises one or more amino acid residue substitutions in one or more of the CDR sequences, and/or one or more of the FR sequences. In certain embodiments, an affinity variant comprises no more than 20, 15, 10, 9, 8, 7, 6, 5, 4, 3, 2, or 1 substitutions in the CDR sequences and/or FR sequences in total.

In certain embodiments, the anti-LILRB4 antibodies or antigen-binding fragments thereof provided herein comprise 1, 2, or 3 CDR sequences having at least 80% (e.g., at least 85%, 88%, 90%, 91%, 92%, 93%, 94%, 95%, 96%, 97%, 98%, or 99%) sequence identity to that (or those) listed in Table 2 or Table 21 above yet retaining the specific binding affinity to LILRB4 at a level similar to or even higher than its parent antibody.

In certain embodiments, the anti-LILRB4 antibodies or antigen-binding fragments thereof comprise one or more variable region sequences having at least 80% (e.g., at least 85%, 88%, 90%, 91%, 92%, 93%, 94%, 95%, 96%, 97%, 98%, or 99%) sequence identity to that (or those) listed in Table 3 or Table 21 above yet retaining the specific binding affinity to LILRB4 at a level similar to or even higher than its parent antibody. In some embodiments, a total of 1 to 10 amino acids have been substituted, inserted, or deleted in a variable region sequence listed in Table 3 or Table 21 above. In some embodiments, the substitutions, insertions, or deletions occur in regions outside the CDRs (e.g., in the FRs).

### Glycosylation Variants

The anti-LILRB4 antibodies or antigen-binding fragments thereof provided herein also encompass glycosylation variants, which can be obtained to either increase or decrease the extent of glycosylation of the antibodies or antigen-binding fragments thereof.

The antibodies or antigen-binding fragments thereof provided herein may comprise one or more modifications that introduce or remove a glycosylation site. A glycosylation site is an amino acid residue with a side chain to which a carbohydrate moiety (e.g., an oligosaccharide structure) can be attached. Glycosylation of antibodies is typically either N-linked or O-linked. N-linked refers to the attachment of the carbohydrate moiety to the side chain of an asparagine residue, for example, an asparagine residue in a tripeptide sequence such as asparagine-X-serine and asparagine-X-threonine, where X is any amino acid except proline. O-linked glycosylation refers to the attachment of one of the sugars N-aceylgalactosamine, galactose, or xylose to a hydroxyamino acid, most commonly to serine or threonine. Removal of a native glycosylation site can be conveniently accomplished, for example, by altering the amino acid sequence such that one of the above-described tripeptide sequences (for N-linked glycosylation sites) or serine or threonine residues (for O-linked glycosylation sites) present in the sequence is substituted. A new glycosylation site can be created in a similar way by introducing such a tripeptide sequence or serine or threonine residue.

### Cysteine-Engineered Variants

The anti-LILRB4 antibodies or antigen-binding fragments thereof provided herein also encompass cysteine-engineered variants, which comprise one or more introduced free cysteine amino acid residues.

A free cysteine residue is one which is not part of a disulfide bridge. A cysteine-engineered variant is useful for conjugation with for example, a cytotoxic and/or imaging compound, a label, or a radioisoptype among others, at the site of the engineered cysteine, through for example a maleimide or haloacetyl. Methods for engineering antibodies or antigen-binding fragments thereof to introduce free cysteine residues are known in the art, see, for example, WO2006/034488.

### Fc Variants

The anti-LILRB4 antibodies and antigen-binding fragments provided herein also encompass an Fc variant, which comprises one or more amino acid residue modifications or substitutions at its Fc region and/or hinge region.

In certain embodiments, the anti-LILRB4 antibodies or antigen-binding fragments thereof provided herein comprise one or more amino acid substitution(s) that improves pH-dependent binding to neonatal Fc receptor (FcRn). Such a variant can have an extended pharmacokinetic half-life, as it binds to FcRn at acidic pH which allows it to escape from degradation in the lysosome and then be translocated and released out of the cell. Methods of engineering an antibody or antigen-binding fragment thereof to improve binding affinity with FcRn are well-known in the art, see, for example, Vaughn, D. et al., Structure, 6(1): 63-73, 1998; Kontermann, R. et al., Antibody Engineering, Volume 1, Chapter 27: Engineering of the Fc region for improved PK, published by Springer, 2010; Yeung, Y. et al., Cancer Research, 70: 3269-3277 (2010); and Hinton, P. et al., J. Immunology, 176:346-356 (2006).

In certain embodiments, the anti-LILRB4 antibodies or antigen-binding fragments thereof provided herein comprise one or more amino acid substitution(s) that alters ADCC. Certain amino acid residues at CH2 domain of the Fc region can be substituted to provide for enhanced ADCC activity. Alternatively or additionally, carbohydrate structures on the antibody can be changed to enhance ADCC activity. Methods of altering ADCC activity by antibody engineering have been described in the art, see for example, Shields R L. et al., J Biol Chem. 2001. 276 (9): 6591-604; Idusogie E E. et al., J Immunol. 2000.164 (8): 4178-84; Steurer W. et al., J Immunol. 1995, 155 (3): 1165-74; Idusogie EE. et al., J Immunol. 2001, 166 (4): 2571-5; Lazar G A. et al., PNAS, 2006, 103 (11): 4005-4010; Ryan M C. et al., Mol. Cancer Ther., 2007, 6:3009-3018; Richards J O, et al., Mol Cancer Ther. 2008, 7 (8): 2517-27; Shields R. L. et al., J. Biol. Chem, 2002, 277:26733-26740; Shinkawa T. et al., J. Biol. Chem, 2003, 278:3466-3473.

In certain embodiments, the anti-LILRB4 antibodies or antigen-binding fragments thereof comprise one or more amino acid substitution(s) that alters CDC, for example, by improving or diminishing C1q binding and/or CDC (see, for example, WO99/51642; Duncan & Winter Nature 322:738-40 (1988); U.S. Pat. Nos. 5,648,260; 5,624,821); and WO94/29351 concerning other examples of Fc region variants. One or more amino acids selected from amino acid residues 329, 331 and 322 of the Fc region can be replaced with a different amino acid residue to alter C1q binding and/or enhance CDC (see, U.S. Pat. No. 6,194,551 by Idusogie et al.). One or more amino acid substitution(s) can also be introduced to alter the ability of the antibody to fix complement (see PCT Publication WO 94/29351 by Bodmer et al.).

In certain embodiments, the anti-LILRB4 antibodies or antigen-binding fragments thereof provided herein comprise one or more amino acid substitution(s) in human immunoglobulin (e.g., IgG1) at position 234 and/or 235 (according to EU numbering). In certain embodiments, the anti-LILRB4 antibodies or antigen-binding fragments thereof provided herein comprise two amino acid substitutions in human immunoglobulin (e.g., IgG1) at positions 234 and 235 (according to EU numbering). In certain embodiments, the anti-LILRB4 antibodies or antigen-binding fragments thereof provided herein comprise L234A and L235A (according to EU numbering) amino acid substitutions.

In certain embodiments, the anti-LILRB4 antibodies or antigen-binding fragments thereof provided herein comprise one or more amino acid substitution(s) in the interface of the Fc region to facilitate and/or promote heterodimerization. These modifications comprise introduction of a protuberance into a first Fc polypeptide and a cavity into a second Fc polypeptide, wherein the protuberance can be positioned in the cavity so as to promote interaction of the first and second Fc polypeptides to form a heterodimer or a complex. Methods of generating antibodies with these modifications are known in the art, e.g., as described in U.S. Pat. No. 5,731,168.

### Antigen-Binding Fragments

Provided herein are also anti-LILRB4 antigen-binding fragments. Various types of antigen-binding fragments are known in the art and can be developed based on the anti-LILRB4 antibodies provided herein, including for example, the exemplary antibodies whose CDRs are shown in Table 2 or Table 21 above, and variable sequences are shown in Table 3 or Table 21 above, and their different variants (such as affinity variants, glycosylation variants, Fc variants, cysteine-engineered variants and so on).

In certain embodiments, an anti-LILRB4 antigen-binding fragment provided herein is a diabody, a Fab, a Fab′, a F(ab′) 2, a Fd, an Fv fragment, a disulfide stabilized Fv fragment (dsFv), a (dsFv) 2, a bispecific dsFv (dsFv-dsFv′), a disulfide stabilized diabody (ds diabody), a single-chain antibody molecule (scFv), an scFv dimer (bivalent diabody), a camelized single domain antibody, a nanobody, a domain antibody, or a bivalent domain antibody.

Various techniques can be used for the production of such antigen-binding fragments. Illustrative methods include, enzymatic digestion of intact antibodies (see, e.g., Morimoto et al., Journal of Biochemical and Biophysical Methods 24:107-117 (1992); and Brennan et al., Science, 229:81 (1985)), recombinant expression by host cells such as E. coli (e.g., for Fab, Fv and ScFv antibody fragments), screening from a phage display library as discussed above (e.g., for ScFv), and chemical coupling of two Fab′-SH fragments to form F(ab′)₂ fragments (Carter et al., Bio/Technology 10:163-167 (1992)). Other techniques for the production of antibody fragments will be apparent to a person skilled in the art.

In certain embodiments, the antigen-binding fragment is a scFv. Generation of scFv is described in, for example, WO 93/16185; U.S. Pat. Nos. 5,571,894; and 5,587,458. ScFv may be fused to an effector protein at either the amino or the carboxyl terminus to provide for a fusion protein (see, for example, Antibody Engineering, ed. Borrebaeck).

In certain embodiments, the anti-LILRB4 antibodies or antigen-binding fragments thereof provided herein are bivalent, tetravalent, hexavalent, or multivalent. Any molecule being more than bivalent is considered multivalent, encompassing for example, trivalent, tetravalent, hexavalent, and so on.

A bivalent molecule can be monospecific if the two binding sites are both specific for binding to the same antigen or the same epitope. This, in certain embodiments, provides for stronger binding to the antigen or the epitope than a monovalent counterpart. Similar, a multivalent molecule may also be monospecific. In certain embodiments, in a bivalent or multivalent antigen-binding moiety, the first valent of binding site and the second valent of binding site are structurally identical (i.e., having the same sequences), or structurally different (i.e., having different sequences albeit with the same specificity).

A bivalent can also be bispecific, if the two binding sites are specific for different antigens or epitopes. This also applies to a multivalent molecule. For example, a trivalent molecule can be bispecific when two binding sites are monospecific for a first antigen (or epitope) and the third binding site is specific for a second antigen (or epitope).

### Bispecific or Multi-Specific Antibodies

In certain embodiments, the anti-LILRB4 antibody or antigen-binding fragment thereof provided herein is bispecific or multi-specific. In certain embodiments, the anti-LILRB4 antibody or antigen-binding fragment thereof provided herein is further linked to a second functional moiety having a different binding specificity from said anti-LILRB4 antibody or antigen-binding fragment thereof. In some embodiments, the bispecific or multi-specific antibody or antigen-binding fragment thereof provided herein has a first specificity for LILRB4, and a second specificity. In some embodiments, the second specificity is for LILRB4 but to different epitopes. In some embodiments, the second specificity is for a second antigen different from LILRB4.

In certain embodiments, the second specificity is for a tumor associated antigen or an epitope thereof. The term “tumor associated antigen” refers to an antigen that is or can be presented on a tumor cell surface and that is located on or within tumor cells. In some embodiments, the tumor associated antigens can be presented only by tumor cells and not by normal cells, i.e., non-tumor cells. In some other embodiments, the tumor associated antigens can be exclusively expressed on tumor cells or may represent a tumor specific mutation compared to non-tumor cells. In some other embodiments, the tumor associated antigens can be found in both tumor cells and non-tumor cells, but are overexpressed on tumor cells when compared to non-tumor cells or are accessible for antibody binding in tumor cells due to the less compact structure of the tumor tissue compared to non-tumor tissue. In some embodiments, the tumor associated antigen is located on the vasculature of a tumor.

In certain embodiments, the bispecific or multi-specific antibodies or antigen-binding fragments thereof provided herein are capable of binding to one or more (e.g., 1, 2, 3, 4, 5 or more) additional antigens other than LILRB4, or a second epitope on LILRB4. In certain embodiments, the one or more additional antigens other than LILRB4 are selected from the group consisting of CD3, CD16a, CD33, CD38, CD45, CD123, CD146, CD228, CLL-1, Flt3, TAF1, TgPRF, HVCN1, IL-6R, IL-11R, IL17A, IL-23R, IL-33, ILDR2, LAP, TSLP, TREM-1, ANGPT2, APOE, IFNAR, CypA, DOG-1, NKp30, CSF-1R, CCR2, LRRC15, mesothelin, Dickkopf2, DLL3, HER-2, C10orf54, TrkA, MEKK1, KRAS, ERK, XPO1, mTORC1/2, PAK4, NAMPT, ATR, EGFR, FGFR, VEGF, c-MET, Her2, Her3, CTLA4, GITA, CD112R, CD2, CD7, CD16, CD19, CD20, CD24, CD27, CD30, CD34, CD37, CD39, CD70, CD73, CD83, CD28, CD80 (B7-1), CD86 (B7-2), CD40, CD40L (CD154), CD47, SIRPα, CD122, CD137, CD137L, OX40 (CD134), OX40L (CD252), BCMA (e.g., BCMA02), PSMA, CLDN18 (e.g., CLDN18.2), NKG2C, 4-1BB, LIGHT, PVRIG, SLAMF7, HVEM, BAFFR, ICAM-1, 2B4, LFA-1, GITR, ICOS (CD278), ICOSLG (CD275), LAG3 (CD223), A2AR, B7-H3 (CD276), B7-H4 (VTCN1), B7-H5, BTLA (CD272), BTLA, CD160, CTLA-4 (CD152), GPRC5D, IDO1, IDO2, ILT3, TDO, KIR, LAIR-1, NOX2, PD-1, PD-L1, PD-L2, TIM-3, VISTA, SIGLEC-7 (CD328), SIGLEC-9 (CD329), SIGLEC-15, TIGIT, PVR (CD155), LILRB2, LILRB3, FLT3, FLT3L, TLR3, CLEC9A, DEC-205, STING, IL-12, IDO, and TGFβ.

In certain embodiments, the bispecific antibodies or antigen-binding fragments thereof provided herein bind to LILRB4 and CD3. In certain embodiments, the bispecific antibodies or antigen-binding fragments thereof provided herein bind to LILRB4 and BCMA. In certain embodiments, the bispecific antibodies or antigen-binding fragments thereof provided herein bind to LILRB4 and CD38. In certain embodiments, the bispecific antibodies or antigen-binding fragments thereof provided herein bind to LILRB4 and CD19. In certain embodiments, the bispecific antibodies or antigen-binding fragments thereof provided herein bind to LILRB4 and FcRH5. In certain embodiments, the bispecific antibodies or antigen-binding fragments thereof provided herein bind to LILRB4 and PD-L1. In certain embodiments, the bispecific antibodies or antigen-binding fragments thereof provided herein bind to LILRB4 and PD-1. In certain embodiments, the bispecific antibodies or antigen-binding fragments thereof provided herein bind to LILRB4 and TIM-3. In certain embodiments, the bispecific antibodies or antigen-binding fragments thereof provided herein bind to LILRB4 and FLT3. In certain embodiments, the bispecific antibodies or antigen-binding fragments thereof provided herein bind to LILRB4 and FLT3L. In certain embodiments, the bispecific antibodies or antigen-binding fragments thereof provided herein bind to LILRB4 and TLR3. In certain embodiments, the bispecific antibodies or antigen-binding fragments thereof provided herein bind to LILRB4 and PD-L1. In certain embodiments, the bispecific antibodies or antigen-binding fragments thereof provided herein bind to LILRB4 and LILRB3. In certain embodiments, the bispecific antibodies or antigen-binding fragments thereof provided herein bind to LILRB4 and VISTA. In certain embodiments, the bispecific antibodies or antigen-binding fragments thereof provided herein bind to LILRB4 and TIGIT. In certain embodiments, the bispecific antibodies or antigen-binding fragments thereof provided herein bind to LILRB4 and 4-1BB. In certain embodiments, the bispecific antibodies or antigen-binding fragments thereof provided herein bind to LILRB4 and B7-H3. In certain embodiments, the bispecific antibodies or antigen-binding fragments thereof provided herein bind to LILRB4 and B7-H4.

### Conjugates

In some embodiments, the anti-LILRB4 antibodies or antigen-binding fragments thereof provided herein further comprise one or more conjugate moieties. The conjugate moiety can be linked to the antibodies or antigen-binding fragments thereof. A conjugate moiety is a moiety that can be attached to the antibody or antigen-binding fragment thereof. It is contemplated that a variety of conjugate moieties may be linked to the antibodies or antigen-binding fragments thereof provided herein (see, for example, “Conjugate Vaccines”, Contributions to Microbiology and Immunology, J. M. Cruse and R. E. Lewis, Jr. (eds.), Carger Press, New York, (1989)). These conjugate moieties may be linked to the antibodies or antigen-binding fragments thereof by covalent binding (e.g., disulfide bond), affinity binding, intercalation, coordinate binding, complexation, association, blending, or addition, among other methods. In some embodiments, the antibodies or antigen-binding fragments thereof can be linked to one or more conjugates via a linker or a crosslinking agent. The linker or crosslinking agent comprises a reactive chemical group that can react with the anti-LILRB4 antibodies or fragments thereof. The reactive chemical groups can be N-succinimidyl esters and N-sulfosuccinimidyl esters. Additionally, the linker comprises a reactive chemical group, which can be a dithiopyridyl group that can react with the drug to form a disulfide bond. Linker molecules include, for example, N-succinimidyl 4-(maleimidomethyl) cyclohexanecarboxylate (SMCC), N-succinimidyl 3-(2-pyridyldithio) propionate (SPDP) (see, e.g., Carlsson et al., Biochem. J., 173:723-737 (1978)), N-succinimidyl 4-(2-pyridyldithio) butanoate (SPDB) (see, e.g., U.S. Pat. No. 4,563,304), N-succinimidyl 4-(2-pyridyldithio) 2-sulfobutanoate (sulfo-SPDB) (see US Publication No. 20090274713), N-succinimidyl 4-(2-pyridyldithio) pentanoate (SPP) (see, e.g., CAS Registry number 341498 August 6), 2-iminothiolane, or acetylsuccinic anhydride. For example, the antibody or cell binding agent can be modified with crosslinking reagents and the antibody or cell binding agent containing free or protected thiol groups thus derived is then reacted with a disulfide- or thiol-containing maytansinoid to produce conjugates. The conjugates can be purified by chromatography, including but not limited to HPLC, size-exclusion, adsorption, ion exchange and affinity capture, dialysis or tangential flow filtration.

In certain embodiments, the antibodies or antigen-binding fragments thereof provided herein may be engineered to contain specific sites outside the epitope binding portion that may be utilized for binding to one or more conjugate moieties. For example, such a site may include one or more reactive amino acid residues, such as for example cysteine or histidine residues, to facilitate covalent linkage to a conjugate moiety.

In certain embodiments, the antibodies or antigen-binding fragments thereof provided herein may be linked to a conjugate moiety indirectly, or through another conjugate moiety. For example, the antibodies or antigen-binding fragments thereof provided herein may be conjugated to biotin, then indirectly conjugated to a second conjugate that is conjugated to avidin. In some embodiments, the conjugate moiety comprises a clearance-modifying agent (e.g., a polymer such as PEG which extends half-life), a chemotherapeutic agent, a toxin, a radioactive isotope, a lanthanide, a detectable label (e.g., a luminescent label, a fluorescent label, an enzyme-substrate label), a DNA-alkylator, a topoisomerase inhibitor, a tubulin-binder, a purification moiety or other anticancer drugs (e.g., agonist of toll-like receptor 7 (TLR-7), TLR-8 and/or TLR-9, siRNA, antibody or antigen-binding fragments thereof, a peptide (such as a short peptide), etc.).

A “toxin” can be any agent that is detrimental to cells or that can damage or kill cells. Examples of toxin include, without limitation, taxol, taxoids, CC-1065 and CC-1065 analogs, duocarmycins and duocarmycin analogs, enediynes such as calicheamicins, dolastatin and dolastatin analogs including auristatins, tomaymycin derivatives, leptomycin derivatives, cisplatin, carboplatin, daunorubicin, doxorubicin, vincristine, vinblastine, melphalan, mitomycin C, chlorambucil and morpholino doxorubicin, cytochalasin B, gramicidin D, ethidium bromide, emetine, mitomycin, etoposide, tenoposide, vincristine, MMAE, MMAF, DM1, DM4, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin and analogs thereof, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), anti-mitotic agents (e.g., vincristine and vinblastine), a topoisomerase inhibitor, and a tubulin-binders.

Examples of detectable label may include a fluorescent label (e.g., fluorescein, rhodamine, dansyl, phycoerythrin, or Texas Red), an enzyme-substrate label (e.g., horseradish peroxidase, alkaline phosphatase, luceriferases, glucoamylase, lysozyme, saccharide oxidases or β-D-galactosidase), a radioisotope (e.g., ¹²³I, ¹²⁴I, ¹²⁵I, ¹³¹I, ³⁵S, ³H, ¹¹¹In, ¹¹²In, ¹⁴C, ⁶⁴Cu, ⁶⁷Cu, ⁸⁶Y, ⁸⁸Y, ⁹⁰Y, ¹⁷⁷Lu, ²¹¹At, ¹⁸⁶Re, ¹⁸⁸Re, ¹⁵³Sm, ²¹²Bi, and ³²P, other lanthanides), a luminescent label, a chromophoric moiety, digoxigenin, biotin/avidin, a DNA molecule or gold for detection.

In certain embodiments, the conjugate moiety can be a clearance-modifying agent which helps increase half-life of the antibody. Illustrative examples include water-soluble polymers, such as PEG, carboxymethylcellulose, dextran, polyvinyl alcohol, polyvinyl pyrrolidone, copolymers of ethylene glycol/propylene glycol, and the like. The polymer may be of any molecular weight, and may be branched or unbranched. The number of polymers attached to the antibody may vary, and if more than one polymer are attached, they can be the same or different molecules.

In certain embodiments, the conjugate moiety can be a purification moiety such as a magnetic bead.

In certain embodiments, the antibody or an antigen-binding fragment thereof provided herein is used as a base for a conjugate.

In certain embodiments, the antibody or an antigen-binding fragment thereof provided herein is conjugated to a signal peptide. A signal peptide (sometimes referred to as signal sequence, leader sequence or leader peptide) can be used to facilitate secretion and isolation of the antibodies or antigen-binding fragments thereof provided herein. Signal peptides are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the invention pertains to the described polypeptides having a signal sequence, as well as to polypeptides from which the signal sequence has been proteolytically cleaved (i.e., the cleavage products). In one embodiment, a nucleic acid sequence encoding a signal sequence can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods. Alternatively, the signal sequence can be linked to the protein of interest using a sequence which facilitates purification, such as with a GST domain.

### Chimeric Antigen Receptor

In certain embodiments, the present disclosure provides a chimeric antigen receptor comprising the antibody or an antigen-binding fragment thereof provided herein, a transmembrane region and an intracellular signal region.

The term “chimeric antigen receptor” or “CAR” or “CARs” as used herein refers to engineered receptors, which graft an antigen specificity onto cells (for example, T cells such as naive T cells, central memory T cells, effector memory T cells, regulatory T cells or combination thereof). CARs are also known as artificial T-cell receptors, chimeric T-cell receptors or chimeric immunoreceptors. In some embodiments, CARs comprise an antigen-specific targeting region (for example, the antigen-binding fragments of the anti-LILRB4 antibody as provided herein), an extracellular region, a transmembrane region, one or more co-stimulatory regions, and an intracellular signal region.

In some embodiments, the antigen-specific targeting region is an scFv. In some embodiments, the transmembrane region comprises a transmembrane region of CD3, CD4, CD8 or CD28. In some embodiments, the co-stimulatory region comprises a co-stimulatory domain of CD28, ICOS, CD27, 4-1BB, OX40 and CD40L. In some embodiments, the intracellular signal region is selected from the group consisting of: an intracellular signal region sequence of CD3, FcγRI, CD27, CD28, CD137, CD134, MyD88, CD40, CD278, TLRs, or a combination thereof.

The CARs may be grafted onto various cells, for example, allogeneic cells, autologous cells or xenogeneic cells.

The term “allogeneic cell” as used herein refers to any cells derived from a different subject of the same species.

The term “autologous cell” as used herein refers to any cells derived from the same subject into which they are later to be re-introduced.

The term “xenogeneic cell” as used herein refers to any cells derived from a different subject of a different species.

In some embodiments, the CARs are grafted on immune effector cells, for example, T cells, natural killer cells, macrophage cells, tumor-infiltrating lymphocytes, etc.

### Polynucleotides and Recombinant Methods

The present disclosure provides isolated polynucleotides that encode the antibodies or antigen-binding fragments thereof, and/or the chimeric antigen receptors provided herein. The term “nucleic acid” or “polynucleotide” as used herein refers to deoxyribonucleic acids (DNA) or ribonucleic acids (RNA) and polymers thereof in either single- or double-stranded form. Unless otherwise indicated, a particular polynucleotide sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (see Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

DNA encoding the antibody or antigen-binding fragment thereof provided herein is readily isolated and sequenced using conventional procedures (e.g., by using oligonucleotide probes that are capable of binding specifically to genes encoding the heavy and light chains of the antibody). The encoding DNA may also be obtained by synthetic methods.

The isolated polynucleotide that encodes the antibodies or antigen-binding fragments thereof and/or the chimeric antigen receptors provided herein can be inserted into a vector for further cloning (amplification of the DNA) or for expression, using recombinant techniques known in the art. Many vectors are available. The vector components generally include, but are not limited to, one or more of the following: a signal sequence, an origin of replication, one or more marker genes, an enhancer element, a promoter (e.g., SV40, CMV, EF-1a), and a transcription termination sequence.

The present disclosure provides vectors comprising the isolated polynucleotides provided herein. In certain embodiments, the polynucleotides provided herein encodes the antibodies or antigen-binding fragments thereof and/or the chimeric antigen receptors provided herein, at least one promoter (e.g., SV40, CMV, EF-1a) operably linked to the nucleic acid sequence, and at least one selection marker. Examples of vectors include, but are not limited to, retrovirus (including lentivirus), adenovirus, adeno-associated virus, herpesvirus (e.g., herpes simplex virus), poxvirus, baculovirus, papillomavirus, papovavirus (e.g., SV40), lambda phage, and M13 phage, plasmid pcDNA3.3, pMD18-T, pOptivec, pCMV, pEGFP, pIRES, pQD-Hyg-GSeu, pALTER, pBAD, pcDNA, pCal, pL, pET, pGEMEX, pGEX, pCI, pEGFT, pSV2, pFUSE, pVITRO, pVIVO, pMAL, pMONO, pSELECT, pUNO, pDUO, Psg5L, pBABE, pWPXL, pBI, p15TV-L, pPro18, pTD, pRS10, pLexA, pACT2.2, pCMV-SCRIPT®, pCDM8, pCDNA1.1/amp, pcDNA3.1, pRc/RSV, PCR 2.1, pEF-1, pFB, pSG5, pXT1, pCDEF3, pSVSPORT, pEF-Bos etc.

Vectors comprising the polynucleotide sequence encoding the antibody or antigen-binding fragment thereof and/or the chimeric antigen receptors provided herein can be introduced to a host expression system (e.g., a host cell) for cloning or gene expression. In certain embodiments, the host expression system provided herein is a microorganism, a yeast, or a mammalian cell. In certain embodiments, the microorganism is selected from the group consisting of E. coli and B. subtilis. In certain embodiments, the yeast is Saccharomyces. In certain embodiments, the mammalian cell is selected from the group consisting of COS, CHO-S, CHO-K1, HEK-293, and 3T3 cells.

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

In addition to prokaryotes, eukaryotic microbes such as filamentous fungi or yeast are suitable cloning or expression hosts for anti-LILRB4 antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated antibodies or antigen-fragment thereof provided herein are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruiffly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; mouse forestomach carcinoma cells (MFC), SNU620 cells, and a human hepatoma line (Hep G2). In some embodiments, the host cell is a mammalian cultured cell line, such as CHO, BHK, NS0, 293, MFC, SNU620 and their derivatives.

Host cells are transformed with the above-described expression or cloning vectors for antibody production and cultured in conventional nutrient media modified as appropriate for inducing promoters, selecting transformants, or amplifying the genes encoding the desired sequences. In another embodiment, the antibody may be produced by homologous recombination known in the art. In certain embodiments, the host cell is capable of producing the antibody or antigen-binding fragment thereof provided herein.

The present disclosure also provides a method of expressing the antibody or antigen-binding fragment thereof and/or the chimeric antigen receptors provided herein, comprising culturing the host expression system provided herein under the condition at which the antibody or antigen-binding fragment thereof and/or the chimeric antigen receptor is expressed. The host expression systems used to produce the antibodies or antigen-binding fragments thereof and/or the chimeric antigen receptors provided herein may be cultured in a variety of media. Commercially available media such as Ham's F10 (Sigma), Minimal Essential Medium (MEM) (Sigma), RPMI-1640 (Sigma), and Dulbecco's Modified Eagle's Medium (DMEM) (Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to a person skilled in the art. The culture conditions, such as temperature, pH, and the like, are those previously used with the host cell selected for expression, and will be apparent to a person skilled in the art.

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter et al., Bio/Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibodies or antigen-binding fragments thereof and/or the chimeric antigen receptors prepared from the host expression systems can be purified using, for example, hydroxylapatite chromatography, gel electrophoresis, dialysis, DEAE-cellulose ion exchange chromatography, ammonium sulfate precipitation, salting out, and affinity chromatography, with affinity chromatography being the preferred purification technique.

In certain embodiments, Protein A immobilized on a solid phase is used for immunoaffinity purification of the antibody and antigen-binding fragment thereof and/or the chimeric antigen receptors. The suitability of protein A as an affinity ligand depends on the species and isotype of any immunoglobulin Fc domain that is present in the antibody. Protein A can be used to purify antibodies that are based on human gamma1, gamma2, or gamma4 heavy chains (Lindmark et al., J. Immunol. Meth. 62:1-13 (1983)). Protein G is recommended for all mouse isotypes and for human gamma3 (Guss et al., EMBO J. 5:1567 1575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

### Pharmaceutical Composition

The present disclosure further provides pharmaceutical compositions comprising the antibodies or antigen-binding fragments thereof and/or the chimeric antigen receptors provided herein and one or more pharmaceutically acceptable carriers.

Pharmaceutical acceptable carriers for use in the pharmaceutical compositions disclosed herein may include, for example, pharmaceutically acceptable liquid, gels, or solid carriers, aqueous vehicles, nonaqueous vehicles, antimicrobial agents, isotonic agents, buffers, antioxidants, anesthetics, suspending/dispending agents, sequestering or chelating agents, diluents, adjuvants, excipients, or non-toxic auxiliary substances, other components known in the art, or various combinations thereof.

Suitable components may include, for example, antioxidants, fillers, binders, disintegrants, buffers, preservatives, lubricants, flavorings, thickeners, coloring agents, emulsifiers or stabilizers such as sugars and cyclodextrins. Suitable antioxidants may include, for example, methionine, ascorbic acid, EDTA, sodium thiosulfate, platinum, catalase, citric acid, cysteine, thioglycerol, thioglycolic acid, thiosorbitol, butylated hydroxanisol, butylated hydroxytoluene, and/or propyl gallate. As disclosed herein, inclusion of one or more antioxidants such as methionine in a composition comprising an antibody or antigen-binding fragment thereof and conjugates provided herein decreases oxidation of the antibody or antigen-binding fragment thereof. This reduction in oxidation prevents or reduces loss of binding affinity, thereby improving antibody stability and maximizing shelf-life. Therefore, in certain embodiments, pharmaceutical compositions are provided that comprise one or more antibodies or antigen-binding fragments thereof as disclosed herein and one or more antioxidants such as methionine. Further provided are methods for preventing oxidation of, extending the shelf-life of, and/or improving the efficacy of an antibody or antigen-binding fragment provided herein by mixing the antibody or antigen-binding fragment with one or more antioxidants such as methionine.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcelluose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

The pharmaceutical compositions can be a liquid solution, suspension, emulsion, pill, capsule, tablet, sustained release formulation, or powder. Oral formulations can include standard carriers such as pharmaceutical grades of mannitol, lactose, starch, magnesium stearate, polyvinyl pyrollidone, sodium saccharine, cellulose, magnesium carbonate, etc.

In certain embodiments, the pharmaceutical compositions are formulated into an injectable composition. The injectable pharmaceutical compositions may be prepared in any conventional form, such as for example liquid solution, suspension, emulsion, or solid forms suitable for generating liquid solution, suspension, or emulsion. Preparations for injection may include sterile and/or non-pyretic solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile and/or non-pyretic emulsions. The solutions may be either aqueous or nonaqueous.

In certain embodiments, unit-dose parenteral preparations are packaged in an ampoule, a vial or a syringe with a needle. All preparations for parenteral administration should be sterile and not pyretic, as is known and practiced in the art.

In certain embodiments, a sterile, lyophilized powder is prepared by dissolving an antibody or antigen-binding fragment as disclosed herein in a suitable solvent. The solvent may contain an excipient which improves the stability or other pharmacological components of the powder or reconstituted solution, prepared from the powder. Excipients that may be used include, but are not limited to, water, dextrose, sorbital, fructose, corn syrup, xylitol, glycerin, glucose, sucrose or other suitable agent. The solvent may contain a buffer, such as citrate, sodium or potassium phosphate or other such buffer known to a person skilled in the art at, in one embodiment, about neutral pH. Subsequent sterile filtration of the solution followed by lyophilization under standard conditions known to a person skilled in the art provides a desirable formulation. In one embodiment, the resulting solution will be apportioned into vials for lyophilization. Each vial can contain a single dosage or multiple dosages of the antibody or antigen-binding fragment thereof or composition thereof. Overfilling vials with a small amount above that needed for a dose or set of doses (e.g., about 10%) is acceptable so as to facilitate accurate sample withdrawal and accurate dosing. The lyophilized powder can be stored under appropriate conditions, such as at about 4° C. to room temperature.

Reconstitution of a lyophilized powder with water for injection provides a formulation for use in parenteral administration. In one embodiment, for reconstitution the sterile and/or non-pyretic water or other liquid suitable carrier is added to lyophilized powder. The precise amount depends upon the selected therapy being given, and can be empirically determined.

### Kits

In certain embodiments, the present disclosure provides a kit comprising the antibody or an antigen-binding fragment thereof provided herein and/or the chimeric antigen receptors provided herein and/or the pharmaceutical composition provided herein. In certain embodiments, the present disclosure provides a kit comprising the antibody or an antigen-binding fragment thereof provided herein and/or the chimeric antigen receptors provided herein and/or the pharmaceutical composition provided herein, and a second therapeutic agent. In certain embodiments, the second therapeutic agent is selected from the group consisting of a chemotherapeutic agent, an anti-cancer drug, radiation therapy agent, an immunotherapy agent, an anti-angiogenesis agent, a targeted therapy agent, a cellular therapy agent, a gene therapy agent, a hormonal therapy agent, an antiviral agent, an antibiotic, an analgesics, an antioxidant, a metal chelator, and cytokines.

Such kits can further include, if desired, one or more of various conventional pharmaceutical kit components, such as, for example, containers with one or more pharmaceutically acceptable carriers, additional containers etc., as will be readily apparent to a person skilled in the art. Instructions, either as inserts or a labels, indicating quantities of the components to be administered, guidelines for administration, and/or guidelines for mixing the components, can also be included in the kit.

### Methods of Use

The present disclosure also provides methods of treating, preventing or alleviating a disease, disorder or condition in a subject, comprising administering to the subject a therapeutically effective amount of the antibody or antigen-binding fragment thereof provided herein, and/or the chimeric antigen receptors provided herein, and/or the pharmaceutical composition provided herein. In certain embodiments, the disease, disorder or condition is a LILRB4-related disease, disorder or condition. In certain embodiments, the subject is human.

In some embodiments, the LILRB4-related disease, disorder or condition is characterized in expressing or over-expressing of LILRB4.

In certain embodiments, the disease, disorder or condition is immune disease, inflammatory disease, cancer or neurological disease. In certain embodiments, the cancer is a LILRB4-expressing cancer. “LILRB4-expressing” cancer as used herein refers to a cancer characterized in expressing LILRB4 protein in a cancer cell, a tumor infiltrating immune cell, or expressing LILRB4 in a cancer cell, a tumor infiltrating immune cell at a level significantly higher than that would have been expected of a normal cell. Various methods can be used to determine the presence and/or amount of LILRB4 in a test biological sample from the subject. For example, the test biological sample can be exposed to an anti-LILRB4 antibody or antigen-binding fragment thereof, which binds to and detects the expressed LILRB4 protein. Alternatively, LILRB4 can also be detected at nucleic acid expression level, using methods such as qPCR, reverse transcriptase PCR, microarray, SAGE, FISH, and the like. In some embodiments, the test sample is derived from a cancer cell or tissue, or tumor infiltrating immune cells. The reference sample can be a control sample obtained from a healthy or non-diseased individual, or a healthy or non-diseased sample obtained from the same individual from whom the test sample is obtained. For example, the reference sample can be a non-diseased sample adjacent to or in the neighborhood of the test sample (e.g., tumor). In certain embodiments, the cancer is a solid tumor or hematologic tumor. In certain embodiments, the cancer is a LILRB4-expressing B cell cancer.

In certain embodiments, the disease, disorder or condition is selected from the group consisting of Kawasaki disease, T. gondii, multiple sclerosis, systematic Lupus erythematosus, lung cancer (e.g., non-small-cell lung cancer (NSCLC), small cell lung cancer (SCLC), adenocarcinoma of the lung, squamous cell carcinoma of the lung, Lewis lung carcinoma, or radiation therapy resistant Lewis lung carcinoma), peritoneal cancer, carcinoid cancer, bone cancer, pancreatic cancer, primitive neuroectodermal tumor, skin cancer, gallbladder cancer, cancer of the head or neck, squamous cell cancer, uterine cancer, ovarian cancer, rectal cancer, prostate cancer, bladder cancer (e.g., urothelial cancer), cancer of the anal region (e.g., anal squamous cell carcinoma), gastric or stomach cancer (e.g., gastrointestinal cancer), esophageal cancer, colon cancer, breast cancer, uterine cancer, liver cancer (e.g., hepatoblastoma, hepatocellular carcinoma/hepatoma, or hepatic carcinoma), cholangiocarcinoma, sarcoma, colorectal cancer, carcinoma of the fallopian tubes, salivary gland carcinoma, carcinoma of the cervix, endometrial or uterine carcinoma, osteosarcoma, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, cancer of the nasopharynx, sarcoma of soft tissue, polycythemia vera, cancer of the urethra, cancer of the penis, cancer of the kidney or ureter (e.g., rhabdoid tumor of the kidney), cutaneous T-cell lymphoma, medulloblastoma, nephroblastoma, myelodysplastic syndrome, chronic and non-chronic myeloproliferative disorder, choroid plexus papilloma, renal cell carcinoma, carcinoma of the renal pelvis, neoplasms of the central nervous system (CNS), soft tissue sarcoma (e.g., rhabdomyosarcoma, fibrosarcoma, Kaposi's sarcoma), spinal axis tumors, glioma (e.g., ependymoma, astrocytoma, anaplastic astrocytoma, oligodendroglioma, eye cancer (e.g., retinoblastoma), brain stem glioma, or mixed glioma such as oligoastrocytoma), brain tumor (e.g., glioblastoma/glioblastoma multiforme (GBM), non-glioblastoma brain tumor, or meningioma), melanoma (e.g., cutaneous or intraocular melanoma), thrombocythemia, mesothelioma, mycosis fungoides, Sezary syndrome, idiopathic myelofibrosis, solitary plasmacytoma, vestibular schwannoma, Ewing's sarcoma, chondrosarcoma, MYH associated polyposis, pituitary adenoma, pediatric cancers such as pediatric sarcomas (e.g., neuroblastoma, rhabdomyosarcoma, and osteosarcoma), hematological cancer, lymphoma, Hodgkin's lymphoma, non-Hodgkin's lymphoma, leukemia (e.g., lymphocytic/lymphoblastic leukemia), chronic or acute leukemia, mast cell leukemia, lymphocytic lymphomas, primary CNS lymphoma, chronic lymphocytic leukemia (CLL), acute lymphocytic leukemia (ALL), chronic myeloid leukemia (CML), acute myeloid leukemia (AML), chronic myelomonocytic leukemia (CMML), chronic lymphoblastic leukemia, acute lymphoblastic leukemia, hairy cell leukemia (HCL), Burkitt's lymphoma (BL), multiple myeloma (e.g., relapsed or refractory multiple myeloma), T or B cell lymphoma, mantle cell lymphoma (MCL) (e.g., relapsed or refractory mantle cell lymphoma), malignant melanoma, diffuse large B cell lymphoma (DLBCL), DLBCL that results from follicular lymphoma, high-grade B-cell lymphoma, primary mediastinal large B-cell lymphoma, follicular lymphoma (FL), and primary mediastinal B-cell lymphoma. In some embodiments, the disease, disorder or condition is acute myeloid leukemia. In some embodiments, the disease, disorder or condition is chronic myelomonocytic leukemia.

In some embodiments, the subject has been identified as having a cancer cell or tumor infiltrating immune cells expressing LILRB4, optionally at a level significantly higher from the level normally found on non-cancer cells.

In another aspect, methods are provided to treat, prevent or alleviate a disease, disorder or condition in a subject that would benefit from modulation of LILRB4 activity, comprising administering to the subject a therapeutically effective amount of the antibody or antigen-binding fragment thereof provided herein, and/or the pharmaceutical composition provided herein. In certain embodiments, the disease, disorder or condition is a LILRB4-related disease, disorder or condition, which is defined above.

The therapeutically effective amount of an antibody or antigen-binding fragment provided herein will depend on various factors known in the art, such as for example body weight, age, past medical history, present medications, state of health of the subject and potential for cross-reaction, allergies, sensitivities and adverse side-effects, as well as the administration route and extent of disease development. Dosages may be proportionally reduced or increased by a person skilled in the art (e.g., physician or veterinarian) as indicated by these and other circumstances or requirements.

In certain embodiments, the antibody or antigen-binding fragment provided herein and/or the chimeric antigen receptors provided herein may be administered at a therapeutically effective dosage of about 0.01 mg/kg to about 100 mg/kg. In certain embodiments, the administration dosage may change over the course of treatment. For example, in certain embodiments the initial administration dosage may be higher than subsequent administration dosages. In certain embodiments, the administration dosage may vary over the course of treatment depending on the reaction of the subject.

Dosage regimens may be adjusted to provide the optimum desired response (e.g., a therapeutic response). For example, a single dose may be administered, or several divided doses may be administered over time.

The antibodies or antigen-binding fragments thereof provided herein and/or the chimeric antigen receptors provided herein may be administered by any route known in the art, for example the administration is through a parenteral route comprising subcutaneous, intraperitoneal, intravenous, intramuscular, or intradermal injection; or a non-parenteral route comprising transdermal, oral, intranasal, intraocular, sublingual, rectal, or topical.

In some embodiments, the antibodies or antigen-binding fragments thereof provided herein and/or the chimeric antigen receptors provided herein may be administered alone or in combination with a therapeutically effective amount of a second therapeutic agent. For example, the antibodies or antigen-binding fragments thereof disclosed herein and/or the chimeric antigen receptors provided herein may be administered in combination with a second therapeutic agent, for example, a chemotherapeutic agent, an anti-cancer drug, a radiation therapy agent, an immunotherapy agent, a targeted therapy agent, a cellular therapy agent, a gene therapy agent, a hormonal therapy agent, an antiviral agent, an antibiotic, an analgesics, an antioxidant, a metal chelator, cytokines, an active agent, an imaging agent, a cytotoxic agent, an angiogenesis inhibitor, a kinase inhibitor, a co-stimulation molecule agonist, a co-inhibition molecule blocker, an adhesion molecule blocker, an anti-cytokine antibody or functional fragment thereof, a detectable label or reporter, an antimicrobial, a gene editing agent, a beta agonist, an viral RNA inhibitor, a polymerase inhibitor, an interferon, or a microRNA.

The term “immunotherapy” as used herein, refers to a type of therapy that stimulates immune system to fight against disease such as cancer or that boosts immune system in a general way. Examples of immunotherapy include, without limitation, checkpoint modulators, adoptive cell transfer, cytokines, oncolytic virus and therapeutic vaccines.

“Targeted therapy” is a type of therapy that acts on specific molecules associated with cancer, such as specific proteins that are present in cancer cells but not normal cells or that are more abundant in cancer cells, or the target molecules in the cancer microenvironment that contributes to cancer growth and survival. Targeted therapy targets a therapeutic agent to a tumor, thereby sparing of normal tissue from the effects of the therapeutic agent.

In certain of these embodiments, an antibody or antigen-binding fragment thereof provided herein, and/or the chimeric antigen receptors provided herein, and/or the pharmaceutical composition provided herein that is administered in combination with one or more additional therapeutic agents may be administered simultaneously with the one or more additional therapeutic agents, and in certain of these embodiments the antibody or antigen-binding fragment thereof and/or the pharmaceutical composition provided herein, and the additional therapeutic agent(s) may be administered as part of the same pharmaceutical composition. However, an antibody or antigen-binding fragment thereof and/or the chimeric antigen receptors provided herein and/or a pharmaceutical composition provided herein administered “in combination” with another therapeutic agent does not have to be administered simultaneously with or in the same composition as the agent. An antibody or antigen-binding fragment thereof, or a chimeric antigen receptor or pharmaceutical composition administered prior to or after another agent is considered to be administered “in combination” with that agent as the phrase is used herein, even if the antibody or antigen-binding fragment, or the pharmaceutical composition or the chimeric antigen receptor, and the second agent are administered via different routes. Where possible, additional therapeutic agents administered in combination with the antibodies or antigen-binding fragments thereof, chimeric antigen receptors or pharmaceutical compositions disclosed herein are administered according to the schedule listed in the product information sheet of the additional therapeutic agent, or according to the Physicians' Desk Reference 2003 (Physicians' Desk Reference, 57th Ed; Medical Economics Company; ISBN: 1563634457; 57th edition (November 2002)) or protocols well known in the art.

The present disclosure further provides method of inactivating LILRB4-expressing cells in vivo or in vitro, comprising contacting the LILRB4-expressing cells with the antibody or antigen-binding fragment thereof provided herein, and/or the chimeric antigen receptors provided herein, and/or the pharmaceutical composition provided herein.

The present disclosure further provides methods of modulating LILRB4 activity in LILRB4-expressing cells, comprising exposing the LILRB4-expressing cells to the antibodies or antigen-binding fragments thereof provided herein, and/or the chimeric antigen receptors provided herein, and/or the pharmaceutical composition provided herein. In some embodiments, the LILRB4-expressing cell is a dendritic cell, monocyte, macrophage, B cell, Treg, progenitor mast cell, endothelial cell, or osteoclast.

In another aspect, the present disclosure provides methods of inducing phagocytosis of target cells in vivo or in vitro, comprising exposing the target cells to the antibodies or antigen-binding fragments thereof provided herein, and/or the chimeric antigen receptors provided herein, and/or the pharmaceutical composition provided herein. In some embodiments, the target cell is an antigen presenting cell, a cancer cell or a cell infected by a pathogen.

In another aspect, the present disclosure provides methods of inducing TNF-α production, comprising exposing the tolerogenic dendritic cells to the antibodies or antigen-binding fragments thereof provided herein, and/or the chimeric antigen receptors provided herein, and/or the pharmaceutical composition provided herein.

In another aspect, the present disclosure provides methods of detecting the presence or amount of LILRB4 in a sample, comprising contacting the sample with the antibodies or antigen-binding fragments thereof provided herein, and/or the chimeric antigen receptors provided herein, and/or the pharmaceutical compositions provided herein, and determining the presence or the amount of LILRB4 in the sample.

In another aspect, the present disclosure provides a method of diagnosing a LILRB4-related disease, disorder or condition in a subject, comprising: a) obtaining a sample from the subject, b) contacting the sample obtained from the subject with the antibody or an antigen-binding fragment thereof provided herein and/or the chimeric antigen receptor provided herein and/or the pharmaceutical composition provided herein; c) determining the presence or amount of LILRB4 in the sample; and d) correlating the presence or the amount of LILRB4 to existence or status of the LILRB4-related disease, disorder or condition in the subject.

In another aspect, the present disclosure provides kits comprising the antibodies or antigen-binding fragments thereof provided herein and/or the chimeric antigen receptors provided herein and/or the pharmaceutical compositions provided herein, optionally conjugated with a detectable moiety, which is useful in detecting LILRB4, optionally recombinant LILRB4, LILRB4 expressed on cell surface, or LILRB4-expressing cells. The kits may further comprise instructions for use.

In another aspect, the present disclosure also provides use of the antibody or antigen-binding fragment thereof provided herein and/or the chimeric antigen receptor provided herein and/or the pharmaceutical composition provided herein in the manufacture of a medicament for treating, preventing or alleviating a LILRB4-related disease, disorder or condition in a subject, in the manufacture of a diagnostic reagent for diagnosing a LILRB4-related disease, disorder or condition.

In another aspect, the present disclosure also provides use of the antibody or antigen-binding fragment thereof provided herein and/or the chimeric antigen receptor provided herein and/or the pharmaceutical composition provided herein in the manufacture of a diagnostic reagent for diagnosing a LILRB4 related disease, disorder or condition.

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. A person skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLES

### Example 1. Antibody Generation

### 1.1. Immunization

To generate antibodies targeting LILRB4, Balb/c and CD1 mice were immunized with human LILRB4 overexpressing cells (CHO-S-hLILRB4) and/or recombinant LILRB4 proteins in each group (as shown in Table 5 below). Immunogens were delivered to mice by intrahepatic (I.H.) and/or intraperitoneal (I.P.) injection. The primary immunization was followed by several boosts until animals developed satisfactory antiserum titers suitable for hybridoma development. Complete Freund's Adjuvant (CFA) was used for primary immunization, and Incomplete Freund's Adjuvant (IFA) was used for subsequent immunization. Test bleeds were performed and evaluated by testing using FACS on CHO-S cell line stably over-expressing human LILRB4 (CHO-S-hLILRB4) or using ELISA with extra-cellular domain of recombinant human LILRB4 protein. The immunization protocols for each group of animals were shown in Tables 6, 7, 8, 9, 10 and 11 below.

TABLE 5
Grouping of Animals

| | | | Animal/ | Group | Dosage/ |
|---|---|---|---|---|---|
| Group | Immunogen | Route | Strain | size | mouse |
| 1 | Recombinant | I.H. | Balb/c | 5 | 50 μg |
| | human LILRB4 | | | | |
| | protein | | | | |
| 2 | Recombinant | I.H. | CD1 | 5 | 50 μg |
| | human LILRB4 | | | | |
| | protein | | | | |
| 3 | Recombinant | I.H., I.P. | Balb/c | 5 | 50 μg, |
| | human LILRB4 | | | | 5 × 10⁶ cells |
| | protein and | | | | |
| | CHO-S-hLILRB4 | | | | |
| | cells | | | | |
| 4 | Recombinant | I.H., I.P. | CD1 | 5 | 50 μg, |
| | human LILRB4 | | | | 5 × 10⁶ cells |
| | protein and | | | | |
| | CHO-S-hLILRB4 | | | | |
| | cells | | | | |
| 5 | Recombinant | I.H., I.P. | Balb/c | 5 | 25 μg, |
| | human LILRB4 | | | | 5 × 10⁶ cells |
| | protein and | | | | |
| | CHO-S-hLILRB4 | | | | |
| | cells | | | | |
| 6 | Recombinant | I.H., I.P. | CD1 | 5 | 25 μg, |
| | human LILRB4 | | | | 5 × 10⁶ cells |
| | protein and | | | | |
| | CHO-S-hLILRB4 | | | | |
| | cells | | | | |

TABLE 6
Immunization Protocol for Group 1

| | |
|---|---|
| Day 0 | Pre-bleed (15-30 μL serum/mouse) |
| | Primary: 50 μg/mouse I.H., CFA |
| Day 14 | Boost 1: 50 μg/mouse I.H., IFA |
| Day 21 | Test Bleed (15-30 μL serum/mouse) (TB1) |
| Day 22 | Test bleed FACS |
| Day 28 | Boost 2: 50 μg/mouse I.H., IFA |
| Day 38 | Test Bleed (15-30 μL serum/mouse) (TB2) |
| Day 38 | Test bleed FACS |
| Day 39 | Data analysis and phase conclusion |
| Day 42 | Pre-fusion (final) Boost, I.H., 50 μg/mouse |

Animals not selected for cell fusion were maintained in cage and may be given additional boost immunizations.

TABLE 7
Immunization Protocol for Group 2

| | |
|---|---|
| Day 0 | Pre-bleed (15-30 μL serum/mouse) |
| | Primary: 50 μg/mouse I.H., CFA |
| Day 14 | Boost 1: 50 μg/mouse I.H., IFA |
| Day 21 | Test Bleed (15-30 μL serum/mouse) (TB1) |
| Day 22 | Test bleed FACS |
| Day 28 | Boost 2: 50 μg/mouse I.H., IFA |
| Day 38 | Test Bleed (15-30 μL serum/mouse) (TB2) |
| Day 38 | Test bleed FACS |
| Day 39 | Data analysis and phase conclusion |
| Day 42 | Pre-fusion (final) Boost, I.H., 50 μg/mouse |

Animals not selected for cell fusion were maintained in cage and may be given additional boost immunizations.

TABLE 8
Immunization Protocol for Group 3

| | |
|---|---|
| Day 0 | Pre-bleed (15-30 μL serum/mouse) |
| | Primary: 50 μg/mouse I.H., CFA |
| Day 14 | Boost 1: 50 μg/mouse I.H., IFA |
| Day 21 | Test Bleed (15-30 μL serum/mouse) (TB1) |
| Day 22 | Test bleed FACS |
| Day 28 | Boost 2: 50 μg/mouse I.H., IFA |
| Day 38 | Test Bleed (15-30 μL serum/mouse) (TB2) |
| Day 38 | Test bleed FACS |
| Day 39 | Data analysis and phase conclusion |
| Day 42 | Pre-fusion (final) Boost, I.P., 5 × 10⁶ cells per mouse |

Animals not selected for cell fusion were maintained in cage and may be given additional boost immunizations.

TABLE 9
Immunization Protocol for Group 4

| | |
|---|---|
| Day 0 | Pre-bleed (15-30 μL serum/mouse) |
| | Primary: 50 μg/mouse I.H., CFA |
| Day 14 | Boost 1: 50 μg/mouse I.H., IFA |
| Day 21 | Test Bleed (15-30 μL serum/mouse) (TB1) |
| Day 22 | Test bleed FACS |
| Day 28 | Boost 2: 50 μg/mouse I.H., IFA |
| Day 38 | Test Bleed (15-30 μL serum/mouse) (TB2) |
| Day 38 | Test bleed FACS |
| Day 39 | Data analysis and phase conclusion |
| Day 42 | Pre-fusion (final) Boost, I.P., 5 × 10⁶ cells per mouse |

Animals not selected for cell fusion were maintained in cage and may be given additional boost immunizations.

TABLE 10
Immunization Protocol for Group 5

| | |
|---|---|
| Day 0 | Pre-bleed (15-30 μL serum/mouse) |
| | Primary: 5 × 10⁶ cells/mouse I.P. |
| Day 14 | Boost 1: 25 μg/mouse I.H., IFA |
| Day 21 | Test Bleed (15-30 μL serum/mouse) (TB1) |
| Day 22 | Test bleed FACS |
| Day 28 | Boost 2: 5 × 10⁶ cells per mouse I.P. |
| Day 38 | Test Bleed (15-30 μL serum/mouse) (TB2) |
| Day 38 | Test bleed FACS |
| Day 39 | Data analysis and phase conclusion |
| Day 39 | Pre-fusion (final) Boost, I.P., 5 × 10⁶ cells per mouse |

Animals not selected for cell fusion were maintained in cage and may be given additional boost immunizations.

TABLE 11
Immunization protocol for Group 6

| | |
|---|---|
| Day 0 | Pre-bleed (15-30 μL serum/mouse) |
| | Primary: 5 × 10⁶ cells/mouse I.P. |
| Day 14 | Boost 1: 25 μg/mouse I.H., IFA |
| Day 21 | Test Bleed (15-30 μL serum/mouse) (TB1) |
| Day 22 | Test bleed FACS |
| Day 28 | Boost 2: 5 × 10⁶ cells per mouse I.P. |
| Day 38 | Test Bleed (15-30 μL serum/mouse) (TB2) |
| Day 38 | Test bleed FACS |
| Day 39 | Data analysis and phase conclusion |
| Day 39 | Pre-fusion (final) Boost, I.P., 5 × 10⁶ cells per mouse |

Animals not selected for cell fusion were maintained in cage and may be given additional boost immunizations.

### 1.2. Hybridoma Generation and Screening

Splenocyte fusions were performed on the mice which responded the best to the immunizations as determined by test bleed FACS and ELISA. The FACS assay was performed on a CHO-S cell line with stable over-expression of human LILRB4 (CHO-S-hLILRB4). In the ELISA assay, the extra-cellular domain of recombinant human LILRB4 protein was coated as the ligand to test the antiserum concentration. The lymphocytes from spleens and lymph nodes were fused to a mouse myeloma cell line (SP2/0) using an optimized electrofusion protocol. Multiple fusions were performed to ensure success of the project.

The fusion was plated (2×10⁴ to 10⁵ cells per well) into a stack of 96-well plates. Plates were monitored for growth and fed weekly. Wells with cell growth were screened by primary screening assays in 10-14 days with FACS and/or other feasible assays such as ELISA. Multiple fusions for each targeting antigen were performed and screened. The positive parental clones which showed positive binding with 293F-LILRB4 and positive ELISA signal from primary screening were expanded into 24-well plates for secondary screening.

Following primary screening, positive parental clones expanded into 24-well plates were screened again by the assay described in the FACS or ELISA assay below. Hybridomas of interest were chosen to proceed to subcloning.

The parental hybridomas with desired reactivity and isotypes from the screening funnel above were then subcloned by multiple rounds of limiting dilution or single cell sorting until monoclones were obtained.

The subcloning plates were screened by protein or cell-based ELISA, and the subclones with good binding ability were expanded to 24-wells for confirmation tests. The specificity and cross-reactivity of these subclones were confirmed with FACS analysis. Briefly, parental 293F cells and 293F-hLILRB4 were incubated with antibodies produced by each subclone, respectively. Fluorescent dye-conjugated secondary antibody was used to detect the binding of the primary antibody with the cells. Median fluorescence intensity (MFI) was measured by FACS analysis.

The desired subclonal cell lines were sequenced and further expanded into culture flasks for cryopreservation. 4-6 vials per cell line at 0.5-13.0×10⁶ cells/vial were initially cryopreserved. Master cell bank and working cell bank were established for the selected most valuable cell lines.

As results shown in Table 12, a total of 28 antibodies were discovered with unique sequences that showing positive binding with 293F cell stably over-expressing human LILRB4 protein (293F-hLILRB4), while they showed no binding with parental 293F cells. Among which, 3 antibodies (i.e., 42-F5-B12-F2, 44-F10-B6-D4 and 42-A10-C1-E10) could also bind with cynomolgus (Macaca fascicularis) LILRB4. The MFI of the mouse antibodies staining 293F-hLILRB4, 293F-hLILRB1, 293F-hLILRB2, 293F-hLILRB3, 293F-hLILRB5, 293F-hLILRA1, 293F-hLILRA2, 293F-hLILRA3, 293F-hLILRA4, 293F-hLILRA5, CHO-S-mLILRB4, CHO-S-Macaca fascicularis LILRB4 (CHO-S-cynoLILRB4), detected by FACS were summarized in the Tables 12 and 13 below.

TABLE 12
MFI of antibodies binding with cell lines expressing different
human LILRA family and human LILRB family proteins
hLILRB-family Binding (FACS-MFI)

| | Cell line | | | | |
|---|---|---|---|---|---|
| | 293F- | 293F- | 293F- | 293F- | 293F- |
| Clone No. | hLILRB4 | hLILRB1 | hLILRB2 | hLILRB3 | hLILRB5 |
| Blank | 27.7 | 22.3 | 28.9 | 23.9 | 21.8 |
| Isotype | 30.2 | 28.4 | 38.1 | 25.2 | 22.9 |
| (mIgG1) | | | | | |
| 2-G4-G8-E10 | 778 | 19.6 | 25.8 | 19.8 | 20.4 |
| 36-F3-E7-H9 | 2002 | 21 | 27 | 25.8 | 19.4 |
| 26-H9-B9-B7 | 441 | 20.8 | 28.8 | 23.6 | 19.6 |
| 27-D11- | 588 | 20.8 | 28.3 | 19.7 | 18.8 |
| C2-A10 | | | | | |
| 32-F9-C9-B9 | 339 | 21.1 | 27.2 | 18.9 | 18.9 |
| 27-F11- | 512 | 21.1 | 27.7 | 24.9 | 19.5 |
| E10-G10 | | | | | |
| 27-G5-E9-B8 | 213 | 20.2 | 25.4 | 23.7 | 18.7 |
| 4-E6-E4-B12 | 1529 | 23.6 | 58 | 27.1 | 21.4 |
| 25-G10-C5-G2 | 969 | 27.5 | 94.8 | 33.4 | 28.2 |
| 42-F5-B12-F2 | 1141 | 26.1 | 34.2 | 26.9 | 21.3 |
| 7-B4-C1-C9 | 3954 | 27.7 | 32.2 | 26.1 | 17.8 |
| 36-F4-H1-D9 | 898 | 25.7 | 26.5 | 23.5 | 17 |
| 10-H9-E3-G3 | 210 | 21 | 29.5 | 22.7 | 21.8 |
| 44-F10-B6-D4 | 84.9 | 38.4 | 38.2 | 31.6 | 26.6 |
| 25-E7-A10-H6 | 759 | 22.5 | 33.2 | 23.1 | 16.3 |
| 25-G9-G4-C12 | 383 | 26 | 32.8 | 25.2 | 22.9 |
| 10-G3-B3-E7 | 432 | 26.2 | 34 | 25.1 | 17.4 |
| 42-A10- | 403 | 26.3 | 34.8 | 28.1 | 19 |
| C1-E10 | | | | | |
| 8-B3-F6-H8 | 1065 | 19.4 | 29.5 | 24.6 | 16.8 |
| 48-E11-H7-B5 | 2759 | 27.3 | 45.8 | 26.7 | 18.9 |
| 43-D12- | 727 | 22.6 | 30.4 | 1076 | 17.9 |
| F3-G11 | | | | | |
| 28-F10-B2-F6 | 1709 | 26.1 | 32.2 | 29.2 | 19.3 |
| 2-H1-D7- | 915 | 25.2 | 31.5 | 23 | 22.6 |
| E5-D5 | | | | | |
| 2-H8-C9-F7-E2 | 1709 | 25.4 | 28.8 | 26.7 | 21.6 |
| 42-C8-A12- | 1293 | 29.6 | 34.5 | 602 | 22.2 |
| F3-D11 | | | | | |
| 26-C11-F8- | 1969 | 23.7 | 32.7 | 26.6 | 20.6 |
| B2-D3 | | | | | |
| 7-A7-F9-D8 | 1141 | 27.5 | 30.5 | 26.6 | 19.6 |
| 41-E12-E10-D8 | 566 | 35 | 34.6 | 37.4 | 27.4 |

hLILRA-family Binding (FACS-MFI)

| | Cell line | | | | |
|---|---|---|---|---|---|
| | 293F- | 293F- | 293F- | 293F- | 293F- |
| Clone No. | hLILRA1 | hLILRA2 | hLILRA3 | hLILRA4 | hLILRA5 |
| Blank | 20.8 | 19.1 | 19.1 | 21.2 | 20.8 |
| Isotype | 22.6 | 19.9 | 20.7 | 22.6 | 26.6 |
| (mIgG1) | | | | | |
| 2-G4-G8-E10 | 17.5 | 16.5 | 18.6 | 21 | 54.3 |
| 36-F3-E7-H9 | 21.4 | 19.2 | 19.4 | 22.8 | 22.7 |
| 26-H9-B9-B7 | 21.3 | 17.9 | 19.1 | 19.4 | 19.6 |
| 27-D11- | 21.6 | 17.8 | 17.2 | 19.6 | 17.2 |
| C2-A10 | | | | | |
| 32-F9-C9-B9 | 21.3 | 18.4 | 18.7 | 19.8 | 16.8 |
| 27-F11- | 22.1 | 17.3 | 19.1 | 19.7 | 54.2 |
| E10-G10 | | | | | |
| 27-G5-E9-B8 | 24 | 17.7 | 19.5 | 53.5 | 19.1 |
| 4-E6-E4-B12 | 24.4 | 21.9 | 23.7 | 23.2 | 32.6 |
| 25-G10-C5-G2 | 25.6 | 19.3 | 18.8 | 21.7 | 24.9 |
| 42-F5-B12-F2 | 23.7 | 18.2 | 20.4 | 20.7 | 25.3 |
| 7-B4-C1-C9 | 27.5 | 21.6 | 25.2 | 26.2 | 28.2 |
| 36-F4-H1-D9 | 22.1 | 16.5 | 18.6 | 20.7 | 20.3 |
| 10-H9-E3-G3 | 21.6 | 19.9 | 18.3 | 20.7 | 25 |
| 44-F10-B6-D4 | 25.6 | 24.8 | 22.5 | 24.4 | 26.8 |
| 25-E7-A10-H6 | 23.1 | 21 | 20.1 | 19.9 | 19.9 |
| 25-G9-G4-C12 | 24.3 | 21.7 | 18.6 | 22.7 | 25.3 |
| 10-G3-B3-E7 | 25.7 | 20 | 19.8 | 23.8 | 27.5 |
| 42-A10- | 26.8 | 19.7 | 96.7 | 21.4 | 25.7 |
| C1-E10 | | | | | |
| 8-B3-F6-H8 | 22.2 | 19.6 | 20 | 19.5 | 24.5 |
| 48-E11-H7-B5 | 25.1 | 22.6 | 24.1 | 24.3 | 359 |
| 43-D12- | 22.2 | 20 | 20.2 | 21.2 | 172 |
| F3-G11 | | | | | |
| 28-F10-B2-F6 | 23.9 | 18.5 | 22.3 | 22.8 | 23.6 |
| 2-H1-D7- | 21.9 | 20 | 20 | 23.7 | 24.7 |
| E5-D5 | | | | | |
| 2-H8-C9-F7-E2 | 22.6 | 19.7 | 21.7 | 23 | 22.6 |
| 42-C8-A12- | 27.3 | 28.1 | 24.5 | 27.2 | 27.9 |
| F3-D11 | | | | | |
| 26-C11-F8- | 21.5 | 17.9 | 18.7 | 21 | 25.5 |
| B2-D3 | | | | | |
| 7-A7-F9-D8 | 21.5 | 19.3 | 18.5 | 19.7 | 23.7 |
| 41-E12-E10-D8 | 26.9 | 23 | 24.5 | 92.2 | 27.1 |

TABLE 13
FACS MFI of antibodies binding with cynomolgus
(Macaca fascicularis) LILRB4 cell line

| | FACS MFI (CHO-S- |
|---|---|
| Clone | cynoLILRB4) |
| Blank | 37.6 |
| Isotype (mlgG1) | 40.6 |
| 2-G4-G8-E10 | / |
| 36-F3-E7-H9 | / |
| 26-H9-B9-B7 | / |
| 27-D11-C2-A10 | / |
| 32-F9-C9-B9 | / |
| 27-F11-E10-G10 | / |
| 27-G5-E9-B8 | / |
| 4-E6-E4-B12 | / |
| 25-G10-C5-G2 | / |
| 42-F5-B12-F2 | 260 |
| 7-B4-C1-C9 | / |
| 36-F4-H1-D9 | / |
| 10-H9-E3-G3 | / |
| 44-F10-B6-D4 | 92.3 |
| 25-E7-A10-H6 | / |
| 25-G9-G4-C12 | / |
| 10-G3-B3-E7 | / |
| 42-A10-C1-E10 | 101 |
| 8-B3-F6-H8 | / |
| 48-E11-H7-B5 | / |
| 43-D12-F3-G11 | / |
| 28-F10-B2-F6 | / |
| 2-H1-D7-E5-D5 | / |
| 2-H8-C9-F7-E2 | / |
| 42-C8-A12-F3-D11 | / |
| 26-C11-F8-B2-D3 | / |
| 7-A7-F9-D8 | / |
| 41-E12-E10-D8 | / |

Note:
“/” indicates no detectable binding.

### Example 2. Antibody Characterization: Binding Affinity

### 2.1. Antibodies

The hybridoma antibody clones 2-G4-G8-E10, 36-F3-E7-H9, 26-H9-B9-B7, 27-D11-C2-A10, 32-F9-C9-B9, 27-F11-E10-G10, 27-G5-E9-B8, 4-E6-E4-B12, 25-G10-C5-G2, 42-F5-B12-F2, 7-B4-C1-C9, 36-F4-H1-D9, 10-H9-E3-G3, 44-F10-B6-D4, 25-E7-A10-H6, 25-G9-G4-C12, 10-G3-B3-E7, 42-A10-C1-E10, 8-B3-F6-H8, 48-E11-H7-B5, 43-D12-F3-G11, 28-F10-B2-F6, 2-H1-D7-E5-D5, 2-H8-C9-F7-E2, 42-C8-A12-F3-D11, 26-C11-F8-B2-D3, 7-A7-F9-D8, and 41-E12-E10-D8 were characterized, and their sequences were identified by the methods below.

### 2.2. Hybridoma Sequencing

Total RNA was isolated from the hybridoma cells following the technical manual of RNAiso Plus (TAKARA Cat #9109). Total RNA was then reverse transcribed into cDNA using isotype-specific anti-sense primers or universal primers following the technical manual of PrimeScript II 1^{st} Strand cDNA synthesis Kit (TAKARA Cat #6210A). The antibody fragments of VH and VL were amplified according to TakaRa Taq™ (Cat #R001A). Amplified antibody fragments were cloned into a standard cloning vector separately. Colony PCR was performed to screen for clones with inserts of correct sizes. No less than five colonies with inserts of correct sizes were sequenced for each fragment. The sequences of different clones were aligned and the consensus sequence of these clones was provided.

The variable region sequences of the hybridoma antibodies are provided herein in Table 3 above.

2.3. Antibodies Binding Affinity on 293F-hLILRB4 Cell Lines

To evaluate the binding affinity of antibody candidates to hLILRB4 in a cell-based assay, sequences of 17 mouse antibodies from Table 12 were selected to generate and produce human IgG1 chimeric antibodies. Two benchmark antibodies, IO-202 and NGM831, were also generated as human IgG1 chimeric antibodies. Generally, DNAs encoding variable regions of the 17 mouse antibodies and benchmark antibodies were synthesized and subcloned into an expression vector where human IgG constant gene was included in advance. The vectors were transfected into mammalian cells for recombinant protein expression and the expressed antibody was purified using protein A affinity chromatography column. The resulting chimeric antibodies are referred to herein as ch27-F11-E10-G10, ch43-D12-F3-G11, ch36-F4-H1-D9, ch8-B3-F6-H8, ch44-F10-B6-D4, ch27-G5-E9-B8, ch48-E11-H7-B5, ch42-C8-A12-F3-D11, ch10-H9-E3-G3, ch7-B4-C1-C9, ch25-E7-A10-H6, ch25-G9-G4-C12, ch2-H1-D7-E5-D5, ch27-D11-C2-A10, ch26-H9-B9-B7, ch4-E6-E4-B12, and ch2-H8-C9-F7-E2, where the prefix “ch” indicates “chimeric”, and the following indicates the hybridoma antibody clone. For example, ch27-F11-E10-G10 indicates that it is a chimeric antibody derived from the hybridoma antibody clone 27-F11-E10-G10. The two benchmark antibodies, which were generated as human IgG1 chimeric antibodies, are still named as IO-202 and NGM831, respectively.

The binding affinity of these chimeric antibodies and benchmark antibodies (i.e., IO-202 and NGM831) with 293F cell stably over-expressing human LILRB4 protein (293F-hLILRB4), was determined by FACS analysis. The binding affinities of these chimeric antibodies and benchmark antibodies to LILRB on different cell lines including: 293F-hLILRB4, 293F-hLILRB1, 293F-hLILRB2, 293F-hLILRB3, 293F-hLILRB5, 293F-hLILRA1, 293F-hLILRA2, 293F-hLILRA3, 293F-hLILRA4, 293F-hLILRA5, CHO-S-mLILRB4, CHO-S-Macaca fascicularis LILRB4 (CHO-S-cynoLILRB4), were determined by FACS. Results were summarized in Table 14 below.

The protocol for FACS analysis is described as follows:
(a) Cells were harvested by centrifugation at 300 g for 3 mins.
(b) The cells were washed for 2 times with FACS buffer by centrifuging at 300 g for 3 mins and the supernatant was discarded.
(c) Cell were resuspended and seeded into assay plate (2×10⁵ cells/well) in 50 μl FACS buffer. 50 μL primary antibody was added to the plates at a serial of final concentration (66.67, 22.22, 7.41, 2.47, 0.82, 0.27, 0.09, 0.03 nM). Cells were incubated with antibodies at 4° C. for 1 hour.
(d) Step (b) was repeated and the cells were resuspended with 100 μl diluted 2^{nd} antibody, incubated at 4° C. for 1 hour in the dark.
(e) Step (b) was repeated and the cells were resuspended with 100 μl FACS buffer. The cells were kept in dark for FACS analysis.

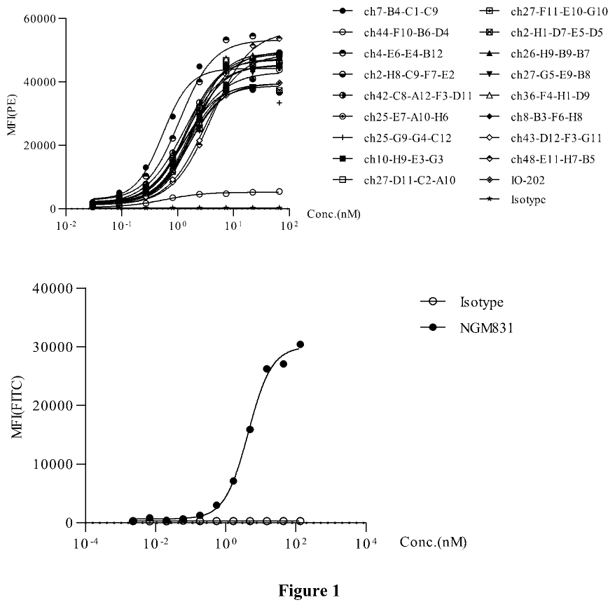

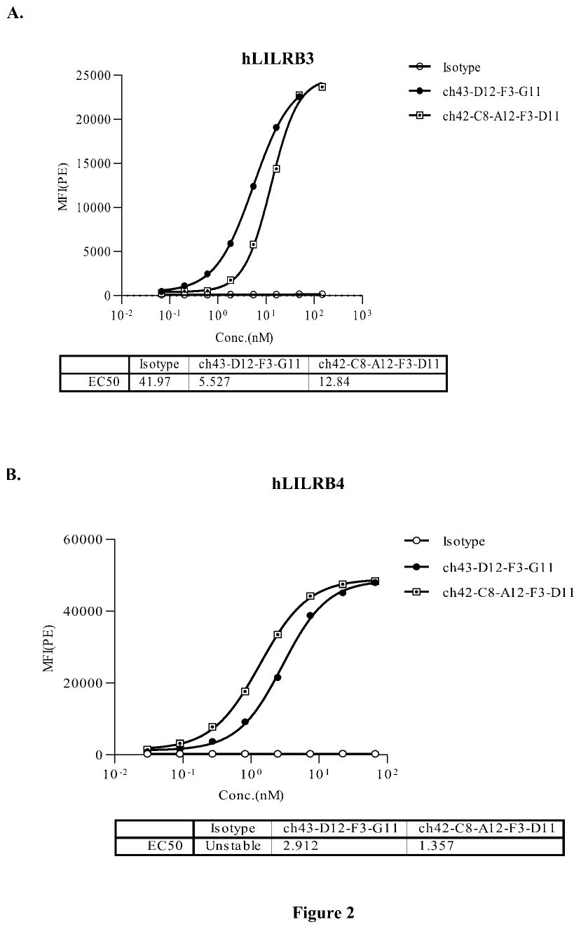

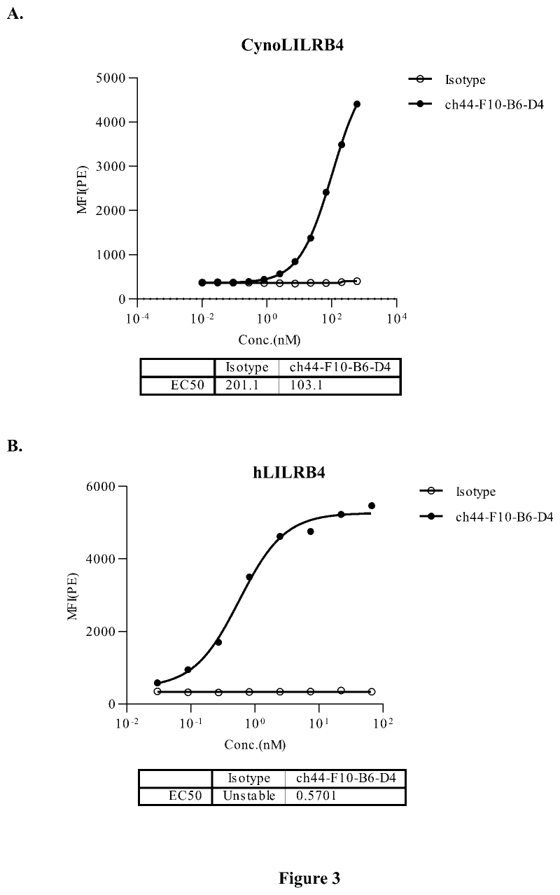

As shown in Table 14 and FIGS. 1, 2 and 3, the binding affinities of the selected chimeric antibodies on 293F-hLILRB4 cell line were higher, lower or comparable with benchmark antibodies IO-202 and NGM831 (Table 14 and FIG. 1). The ch43-D12-F3-G11 and ch42-C8-A12-F3-D11 antibodies showed remarkable cross reactivity with human LILRB3 (FIG. 1 and FIGS. 2A-B) and ch44-F10-B6-D4 showed well cross reactivity with cynomolgus LILRB4 (FIG. 1 and FIGS. 3A-B).

TABLE 14

| Binding affinity of the chimeric antibodies on 293F-hLILRB4 | | |
|---|---|---|
| cell line, 293F-hLILRB3 cell line, CHO-S-cynoLILRB4 | | |
| cell line by FACS and on hLILRA6 protein by ELISA | | |
| | Cell line | |
| | Human | |
| | 293F-hLILRB4 | |
| Clone No. | EC₅₀ (nM) | Max MFI |
| Isotype (hIgG1) | / | 370 |
| IO-202 | 0.016 | 39745 |
| NGM831 | 4.391 | 30447 |
| ch27-F11-E10-G10 | 1.52 | 46240 |
| ch43-D12-F3-G11 | 2.91 | 47982 |
| ch36-F4-H1-D9 | 1.99 | 48241 |
| ch8-B3-F6-H8 | 2.05 | 48471 |
| ch44-F10-B6-D4 | 0.57 | 5467 |
| ch27-G5-E9-B8 | 1.75 | 44283 |
| ch48-E11-H7-B5 | 4.05 | 53698 |
| ch42-C8-A12-F3-D11 | 1.36 | 48355 |
| ch10-H9-E3-G3 | 1.41 | 45167 |
| ch7-B4-C1-C9 | 0.54 | 47333 |
| ch25-E7-A10-H6 | 1.40 | 45015 |
| ch25-G9-G4-C12 | 1.55 | 43275 |
| ch2-H1-D7-E5-D5 | 1.60 | 38324 |
| ch27-D11-C2-A10 | 1.43 | 46762 |
| ch26-H9-B9-B7 | 1.66 | 45989 |
| ch4-E6-E4-B12 | 1.09 | 54499 |
| ch2-H8-C9-F7-E2 | 1.23 | 40996 |
| | Cell line | |
| | Human | |
| | 293F-hLILRB3 | |
| Clone No. | EC₅₀ (nM) | Max MFI |
| Isotype (hIgG1) | / | / |
| IO-202 | / | / |
| NGM831 | / | / |
| ch27-F11-E10-G10 | / | / |
| ch43-D12-F3-G11 | 5.527 | 22554 |
| ch36-F4-H1-D9 | / | / |
| ch8-B3-F6-H8 | / | / |
| ch44-F10-B6-D4 | / | / |
| ch27-G5-E9-B8 | / | / |
| ch48-E11-H7-B5 | / | / |
| ch42-C8-A12-F3-D11 | 12.84 | 23687 |
| ch10-H9-E3-G3 | / | / |
| ch7-B4-C1-C9 | / | / |
| ch25-E7-A10-H6 | / | / |
| ch25-G9-G4-C12 | / | / |
| ch2-H1-D7-E5-D5 | / | / |
| ch27-D11-C2-A10 | / | / |
| ch26-H9-B9-B7 | / | / |
| ch4-E6-E4-B12 | / | / |
| ch2-H8-C9-F7-E2 | / | / |
| | Cell line | |
| | Cynomolgus | |
| | CHO-S-cynoLILRB4 | |
| Clone No. | EC₅₀ (nM) | Max MFI |
| Isotype (hIgG1) | / | / |
| IO-202 | / | 285 |
| NGM831 | / | 822 |
| ch27-F11-E10-G10 | / | / |
| ch43-D12-F3-G11 | / | / |
| ch36-F4-H1-D9 | / | / |
| ch8-B3-F6-H8 | / | / |
| ch44-F10-B6-D4 | 103.1 | 4406 |
| ch27-G5-E9-B8 | / | / |
| ch48-E11-H7-B5 | / | / |
| ch42-C8-A12-F3-D11 | / | / |
| ch10-H9-E3-G3 | / | / |
| ch7-B4-C1-C9 | / | / |
| ch25-E7-A10-H6 | / | / |
| ch25-G9-G4-C12 | / | / |
| ch2-H1-D7-E5-D5 | / | / |
| ch27-D11-C2-A10 | / | / |
| ch26-H9-B9-B7 | / | / |
| ch4-E6-E4-B12 | / | / |
| ch2-H8-C9-F7-E2 | / | / |

| | Cell line |
|---|---|
| | Human |
| | LILRA6 protein |
| Clone No. | ELISA(OD₄₅₀) |
| Isotype (hIgG1) | 0.1522 |
| IO-202 | 0.1566 |
| NGM831 | 1.32 |
| ch27-F11-E10-G10 | / |
| ch43-D12-F3-G11 | 0.4044 |
| ch36-F4-H1-D9 | / |
| ch8-B3-F6-H8 | 0.3668 |
| ch44-F10-B6-D4 | / |
| ch27-G5-E9-B8 | / |
| ch48-E11-H7-B5 | 0.3022 |
| ch42-C8-A12-F3-D11 | 0.3195 |
| ch10-H9-E3-G3 | / |
| ch7-B4-C1-C9 | / |
| ch25-E7-A10-H6 | / |
| ch25-G9-G4-C12 | 0.6866 |
| ch2-H1-D7-E5-D5 | / |
| ch27-D11-C2-A10 | / |
| ch26-H9-B9-B7 | / |
| ch4-E6-E4-B12 | 0.3531 |
| ch2-H8-C9-F7-E2 | 0.5757 |

Note:
“/” indicates no detectable binding.

### 2.4 LILRB4 Expression Pattern on Human Cancer Cell Lines

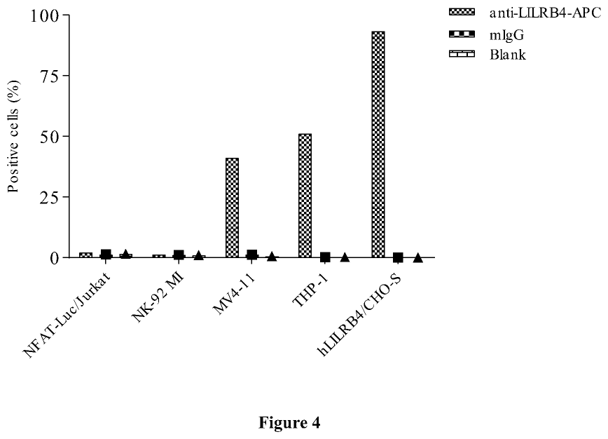

To compare the LILRB4 expression in different human cancer cell lines, human LILRB4 APC-conjugated antibody (Clone: 293623, catalog: FAB24251A-100) was used in the FACS analysis. As shown in FIG. 4, the expression level of LILRB4 was high in some of human cancer cell lines such as THP-1 (Acute myeloid leukemia, AML5) and MV-4-11 (Acute myeloid leukemia, AML5).

### Example 3. Antibody Characterization: Antibody-Dependent Cellular Cytotoxicity (ADCC)

### 3.1. Determine the ADCC of Selected Antibodies

To measure the hLILRB4-specific ADCC efficacy, carboxyfluorescein succinimidyl ester (CFSE) labeled THP-1 cells were used as the target cells that were seeded at 5×10⁴ cells/well in 96-wells Flat-bottom sterile plates, and 2×10⁵ freshly isolated human PBMCs were added as effector cell. Target cells, effector cells (FcR blocked before adding antibodies to CFSE labeled THP-1 cells) and increasing concentrations of antibodies were combined in 200 μL total volume with X-VIVO medium containing 50 ng/ml of IL-2, in U-shaped 96-well plates. Cells and antibodies were incubated at 37° C., 5% CO₂ for 20 hours, following by FACS analysis to measure the percentages of CFSE labeled THP-1 cells. IO-202 and NGM831 were used as the benchmark antibodies (which were generated as human IgG1 chimeric antibodies), while human IgG1 was used as a negative control.

Percent of cell cytotoxicity was calculated according to the equation below:

$$Cell Cytotoxicity ( \% ) = 100 \% - \frac{AT}{NAT} \times 100 \%$$

(AT: percentages of CFSE labeled THP-1 cell in antibody treated group;
NAT: percentages of CFSE labeled THP-1 cell in negative control group)

### 3.2. Results

As shown in Table 15 and FIG. 5, antibody ch2-H1-D7-E5-D5 showed significantly potent ADCC effect on THP-1 cells and lower EC₅₀ compared with benchmark antibodies IO-202 and NGM831.

TABLE 15
Cell cytotoxicity and EC₅₀ of selected antibodies
ADCC effect on THP-1 cells

| | Max cell | |
|---|---|---|
| Clone No. | cytotoxicity (%) | EC₅₀ (nM) |
| Isotype (hIgG1) | 2.75 | / |
| IO-202 | 13.93 | 247.6 |
| NGM831 | −6.89 | 487.7 |
| ch27-F11-E10-G10 | 18.7 | / |
| ch43-D12-F3-G11 | 2.39 | 4.145 |
| ch36-F4-H1-D9 | 17.9 | / |
| ch8-B3-F6-H8 | 7.1 | / |
| ch44-F10-B6-D4 | 0.43 | 1.432 |
| ch27-G5-E9-B8 | 16.4 | / |
| ch48-E11-H7-B5 | / | / |
| ch42-C8-A12-F3-D11 | 14.52 | / |
| ch10-H9-E3-G3 | 16.61 | / |
| ch7-B4-C1-C9 | 13.91 | / |
| ch25-E7-A10-H6 | 11.73 | / |
| ch25-G9-G4-C12 | 11.03 | / |
| ch2-H1-D7-E5-D5 | 39.61 | 7.337 |
| ch27-D11-C2-A10 | 20.2 | / |
| ch26-H9-B9-B7 | 15.17 | / |
| ch4-E6-E4-B12 | 14.56 | / |
| ch2-H8-C9-F7-E2 | 31.11 | 1.377 |

Note:
“/” indicates undetectable.

### Example 4. Antibody Characterization: Antibody-Dependent Cellular Phagocytosis (ADCP)

### 4.1. Determine the ADCP of Selected Antibodies

To measure the hLILRB4-specific ADCP effect, CFSE labeled MV-4-11 and Far-Red labeled M2 macrophages that differentiated from isolated human PBMCs were added as effector cells. IO-202 and NGM831 were used as the benchmark antibodies (which were generated as human IgG1 chimeric antibodies), while human IgG1 was used as a negative control. The ADCP assay was performed as described below:
(a) CD14⁺ monocytes were first isolated from PBMCs by EasySep (STEMCELL Inc).
(b) Monocytes were differentiated into M2 macrophages by culturing for 8 days in X-VIVO 15, with 100 ng/ml rhM-CSF on Day 0 and Day 3, and with 100 ng/ml rhIL10 and rhTGF-on Day 5, respectively.
(c) 2×10⁴ Far-Red labeled M2 macrophages were mixed with various concentrations of antibodies and 2× 104 CFSE labeled MV-4-11 cells, in which FcR was blocked before adding antibodies (Effector:target=1:1).
(d) The mixture was incubated for 2 hours in a CO₂ incubator at 37° C.
(e) Cells were digested using Trypsin (1×) by centrifuging at 500 g for 3 minutes.
(f) The cells were washed 2 times with FACS buffer by centrifuging at 500 g for 3 minutes.
(g) Phagocytosis was evaluated by flow cytometry, and results were reported as the ratio of cells positive for both CFSE and Far-Red to total macrophages in the sample.

### 4.2. Results

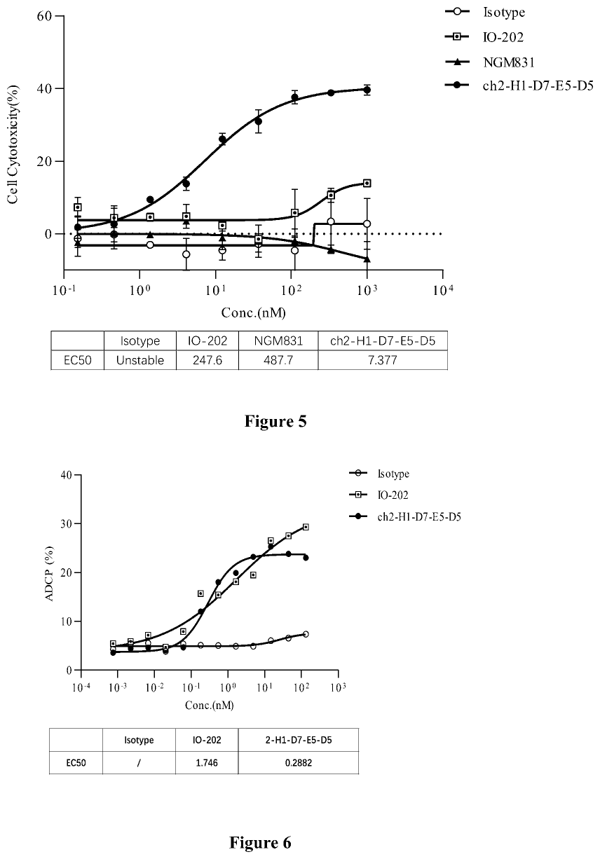

As shown in Table 16 and FIG. 6, all the chimeric antibodies showed potent ADCP effect on MV-4-11 cells. The chimeric antibody ch2-H1-D7-E5-D5 showed lower EC₅₀ compared with benchmark antibody IO-202 (Table 16 and FIG. 6).

TABLE 16
133.3 nM selected antibodies ADCP efficacy
of macrophage and MV-4-11 cells

| Clone | ADCP (%) |
|---|---|
| Isotype (hIgG1) | 2.925 |
| IO-202 | 21.75 |
| NGM831 | 6.37 |
| ch27-F11-E10-G10 | 16.3 |
| ch43-D12-F3-G11 | 18.25 |
| ch36-F4-H1-D9 | 14.75 |
| ch8-B3-F6-H8 | 4.59 |
| ch44-F10-B6-D4 | 3.115 |
| ch27-G5-E9-B8 | 14.8 |
| ch48-E11-H7-B5 | 18 |
| ch42-C8-A12-F3-D11 | 20.4 |
| ch10-H9-E3-G3 | 13.65 |
| ch7-B4-C1-C9 | 11.8 |
| ch25-E7-A10-H6 | 11.03 |
| ch25-G9-G4-C12 | 24.2 |
| ch2-H1-D7-E5-D5 | 27.95 |
| ch27-D11-C2-A10 | 12.05 |
| ch26-H9-B9-B7 | 16.05 |
| ch4-E6-E4-B12 | 14.1 |
| ch2-H8-C9-F7-E2 | 9.35 |

### Example 5. Antibody Characterization: Tolerogenic Dendritic Cell Reprogramming Via Blocking the LILRB4-Fibronectin Interaction

### 5.1. Anti-LILRB4 Antibodies Block LILRB4-Fibronectin Interaction and Reprogram Tolerogenic Dendritic Cells

Dendritic cells (DCs) including monocyte-derived dendritic cells (moDCs) induced by immunosuppressive factors such as TGF-β and IL-10 in the tumor microenvironment is tolerogenic. LILRB4 that contains three cytoplasmic ITIMs (immunoreceptor tyrosine-based inhibitory motifs) in the intracellular domain is markedly overexpressed on tolerogenic dendritic cells. FcγRI (CD64), which is a high affinity IgG receptor, is remarkably expressed on many myeloid cells, such as dendritic cells and macrophages. IgG and FcγRI interaction triggers the intracellular ITAM (immunoreceptor tyrosine-based activating motif) signal of FcγRI to promote TNF-α production. It has been identified that LILRB4 and FcγRI mediated signaling pathway exists crosstalk of LILRB4 and FcγRI, in which LILRB4 regulates FcγRI-mediated ITAM activation to secret TNF-α. Fibronectin is a functional ligand for LILRB4, contributing to unresponsive dendritic cells to the stimulation via FcγRI-IgG interaction. However, FcγRI signaling can be reversed by anti-LILRB4 antibody mediated blockade of fibronectin-LILRB4 interaction. Therefore, the inventors evaluated the blocking ability of the claimed anti-LILRB4 antibodies via this FcγR stimulation assay in tolerogenic moDCs. IO-202 and NGM831 were used as the benchmark antibodies, which were generated as human IgG1 chimeric antibodies. TNF-α secretion from tolerogenic dendritic cells was used as a readout for evaluating FcγR stimulation. The assay was performed as described below:
(a) Day 0: monocytes were isolated from PBMCs and resuspend at 5×10⁶ cells in 3 ml X-VIVO, supplemented with 2 U/ml of dendritic cell culture factor and then culture cells in 6-well plates.
(b) Day 2: Added 2 ml/well of fresh complete medium supplemented with 2 U/ml of dendritic cell culture factor.
(c) Day 5: Immature moDCs were differentiated into tolDC by adding 10 nmol/L dexamethasone and 100 nmol/L vitamin D3.
(d) Day 7:
   (1) 96-well plates (Corning) were co-coated with 5 jug/mL fibronectin and 5 μg/mL human IgG1 in PBS at room temperature for 2 hours.
   (2) Plates were washed twice with PBS and preincubated with RPMI1640 containing 10% FBS for 2 hours.
   (3) Tolerogenic human dendritic cells (5˜7×10⁴ cells/well) were plated on the coated wells in X-VIVO 15 media and incubated with the 5 μg/mL designated antibodies at room temperature for 5-20 minutes.
   (4) Cells in coated wells were mixed well and incubated overnight in a CO₂ incubator at 37° C.
   (5) After overnight incubation at 37° C., culture supernatants were collected, and cytokine secretion was measured by ELISA assay using human TNF-α kit according to the manufacturer's instructions.

### 5.2. Results

As shown in Table 17, all of the 17 chimeric LILRB4 antibodies markedly promoted TNF-α production. This suggested that these LILRB4 antibody candidates possessed potent blockade of the LILRB4-fibronectin interaction to reprogram tolerogenic dendritic cells to be mature dendritic cells, which stimulate T cell activation, proliferation and differentiation. 15 of the 17 antibody candidates except ch43-D12-F3-G11 and ch44-F10-B6-D4 showed more potent blocking or reprograming effect compared with benchmark antibodies IO-202 and NGM831.

TABLE 17
TNFα secretion by antibodies blockade of the LILRB4-
fibronectin interaction in reprogrammed tolerogenic dendritic cells

| | FcγR stimulation | Fold change |
|---|---|---|
| Clone | (TNF-α, pg/mL) | (Compared to isotype) |
| Isotype (hIgG1) | 31.60267 | 1 |
| IO-202 | 237.6962 | 7.52 |
| NGM831 | 260.9293 | 8.26 |
| ch27-F11-E10-G10 | 393.428 | 12.45 |
| ch43-D12-F3-G11 | 215.1308 | 6.81 |
| ch36-F4-H1-D9 | 542.8158 | 17.18 |
| ch8-B3-F6-H8 | 390.5342 | 12.36 |
| ch44-F10-B6-D4 | 125.5927 | 3.97 |
| ch27-G5-E9-B8 | 393.7897 | 12.46 |
| ch48-E11-H7-B5 | 1307.952 | 41.39 |
| ch42-C8-A12-F3-D11 | 307.5348 | 9.73 |
| ch10-H9-E3-G3 | 388.8092 | 12.30 |
| ch7-B4-C1-C9 | 415.0195 | 13.13 |
| ch25-E7-A10-H6 | 389.616 | 12.33 |
| ch25-G9-G4-C12 | 356.2549 | 11.27 |
| ch2-H1-D7-E5-D5 | 324.6467 | 10.27 |
| ch27-D11-C2-A10 | 666.2994 | 21.08 |
| ch26-H9-B9-B7 | 547.2399 | 17.32 |
| ch4-E6-E4-B12 | 266.3273 | 8.43 |
| ch2-H8-C9-F7-E2 | 310.2337 | 9.82 |

### Example 6. Antibody Characterization: Antigen Presentation and Co-Stimulation Ability of Dendritic Cells

### 6.1. LILRB4 Antibodies Reprogram the Antigen Presentation and Co-Stimulation of DCs

To test the capability of LILRB4 antibody candidates in regulating the expression of antigen presentation molecules and co-stimulatory molecules, an assay was performed as described below. IO-202 and NGM831 were used as the benchmark antibodies, which were generated as human IgG1 chimeric antibodies.
(a) Day 0: monocytes were isolated from PBMCs and resuspend at 5×10⁶ cells in 3 ml X-VIVO, supplemented with 2 U/mL of dendritic cell culture factor and then cells were cultured in 6-well plates.
(b) Day 2: Added 2 mL/well of fresh complete medium supplemented with 2 U/ml of dendritic cell culture factor.
(c) Day 5: Immature DCs were differentiated into tolDC by adding 10 nM dexamethasone and 100 nM vitamin D3.
(d) Day 7: Samples were detected by flow cytometry via staining CD86-PE, HLA-DR-FITC and HLA-ABC-APC.

### 6.2. Results

As shown in Table 18, all the chimeric antibodies induced over-expression of antigen presentation molecules (HLA-DR and HLA-ABC) and co-stimulatory molecule (CD86) on DCs. These results indicated that the chimeric antibodies reprogramed tolerogenic DC to activate T cells. Some (e.g., ch7-B4-C1-C9, ch25-E7-A10-H6, ch2-H8-C9-F7-E2, etc.) of the antibodies demonstrated more potent expression-induction effect compared with benchmark antibodies IO-202 and NGM831.

TABLE 18
MFI of Antigen presentation molecules (HLA-DR and HLA-
ABC) and co-stimulation molecule (CD86) of antibodies induced
reprograming effect on dendritic cells

| Clone | CD86 MFI | HLA-DR MFI | HLA-ABC MFI |
|---|---|---|---|
| Isotype (hIgG1) | 1052 | 2805 | 1601 |
| IO-202 | 1650 | 3097 | 1560 |
| NGM831 | 2087 | 3425 | 1603 |
| ch27-F11-E10-G10 | 1578 | 3444 | 1714 |
| ch43-D12-F3-G11 | 1792 | 3771 | 1935 |
| ch36-F4-H1-D9 | 1777 | 3343 | 2027 |
| ch8-B3-F6-H8 | 3148 | 4521 | 2056 |
| ch44-F10-B6-D4 | 1165 | 3121 | 1910 |
| ch27-G5-E9-B8 | 1977 | 3676 | 1641 |
| ch48-E11-H7-B5 | 1564 | 3462 | 2028 |
| ch42-C8-A12-F3-D11 | 1843 | 3629 | 1496 |
| ch10-H9-E3-G3 | 1862 | 3340 | 1631 |
| ch7-B4-C1-C9 | 2153 | 3654 | 2097 |
| ch25-E7-A10-H6 | 2089 | 3375 | 1881 |
| ch25-G9-G4-C12 | 1485 | 3789 | 1898 |
| ch2-H1-D7-E5-D5 | 1927 | 4485 | 2279 |
| ch27-D11-C2-A10 | 1661 | 3854 | 2097 |
| ch26-H9-B9-B7 | 1512 | 3928 | 2139 |
| ch4-E6-E4-B12 | 1623 | 3903 | 1983 |
| ch2-H8-C9-F7-E2 | 2333 | 4250 | 1839 |

### Example 7. Antibody Characterization: THP-1 and T Cell Coculture Assay of Selected Chimeric Antibodies

The chimeric antibodies ch2-H1-D7-E5-D5 and ch8-B3-F6-H8 prepared in Example 2.3 was used for the THP-1 and T cell coculture assay. The protocol for the THP-1 and T cell coculture assay is as follows:
(a) Human T cells (5×10⁴ cells per well) isolated from health donor peripheral blood were placed in the lower chambers of a 96-well transwell plate.
(b) Irradiated THP-1 cells (E:T ratio=1:4) were cultured and treated with indicated antibodies in the upper chamber of transwell inserts (pore size, 3 mm; Thermo Fisher) in U-bottom 96-well plate.
(c) After cultured with anti-CD3/CD28-coated beads (Thermo Fisher) and 50 U/mL rhIL2 in the lower chambers for 5 to 7 days, representative, T cells in lower chambers were photographed using the microscope. T cells were stained with anti-CD3-PE, or anti-CD8-APC/Fire750 and analyzed by flow cytometry.

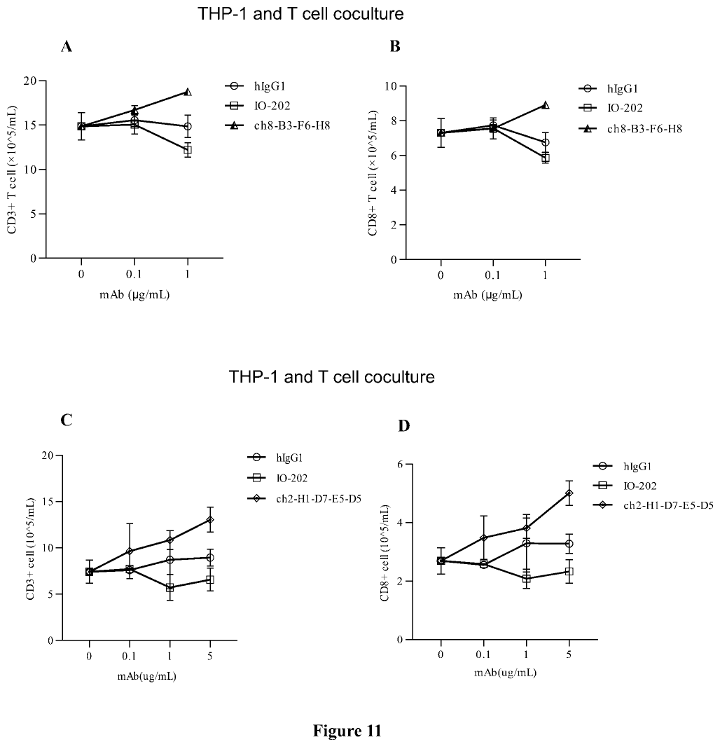

As shown in FIGS. 11A-D, the chimeric antibodies ch2-H1-D7-E5-D5 and ch8-B3-F6-H8 reversed THP-1-mediated T cell suppression.

### Example 8. Antibody Characterization: In Vivo Efficacy Test of Selected Chimeric Antibody in the Treatment of Radiation Therapy Resistant Lewis Lung Carcinoma (LLC1) and EL4-LILRB4 Lymphoma in Female hLILRB1/hLILRB4 Transgenic Mice

The chimeric antibody ch8-B3-F6-H8 prepared in Example 2.3 was used for the in vivo efficacy test in the treatment of radiation therapy resistant Lewis lung carcinoma (LLC1) and EL4-LILRB4 lymphoma in female hLILRB1/hLILRB4 transgenic mice. IO-202 was used as a positive control, and hIgG1 was used as a negative control.

### 8.1. Study Design and Methods

### 8.1.1. Radiation Therapy Resistant LLC1 Model

The protocol for in vivo efficacy assay in the radiation therapy resistant LLC1 model is summarized in Table 19 below and detailed as follows.
(a) The LLC1 tumor cells were maintained in vitro with DMEM medium supplemented with 10% fetal bovine serum at 37° C. in an atmosphere of 5% CO₂ in air. The cells in exponential growth phase were harvested and quantitated by cell counter before tumor inoculation.
(b) Each mouse was inoculated subcutaneously in the right rear flank region with LLC1 tumor cells (3×10\hat{ }6) in 0.1 ml of PBS for tumor development. The date of randomization was denoted as Day 0, dosing starts from Day 0.
(c) The randomization started when the mean tumor size reached approximately 70-100 mm³. 18 mice were enrolled in this study. All animals were randomly allocated to 3 study groups. Randomization were performed based on randomized block design.
(d) Tumor volumes were measured twice per week in two dimensions using a caliper, and the volume were expressed in mm³ using the Formula: V=(L×W×W)/2, where V is tumor volume, L is tumor length (the longest tumor dimension) and W is tumor width (the longest tumor dimension perpendicular to L).
   Dosing as well as tumor and body weight measurements were conducted in a Laminar Flow Cabinet.

TABLE 19
Study design for in vivo efficacy test in radiation therapy resistant
LLC1 model

| | | Tumor | | | Dosing Frequency |
|---|---|---|---|---|---|
| Group | Treatment | model | Dose | ROA | & Duration |
| 1 | hIgG1 | LLC1 | 10 mg/kg | i.p. | Q10D (Day 1, 11), 2 |
| | | | | | doses |
| | Radiation | | 10 Gy | — | Q10D (Day 0, 10) |
| | therapy | | | | |
| 2 | IO-202 | LLC1 | 10 mg/kg | i.p. | Q10D (Day 1, 11), 2 |
| | | | | | doses |
| | Radiation | | 10 Gy | — | Q10D (Day 0, 10) |
| | therapy | | | | |
| 3 | ch8-B3-F6- | LLC1 | 10 mg/kg | i.p. | Q10D (Day 1, 11), 2 |
| | H8 | | | | doses |
| | Radiation | | 10 Gy | — | Q10D (Day 0, 10) |
| | therapy | | | | |

### 8.1.2. EL4-LILRB4 Lymphoma Model

The protocol for in vivo efficacy assay in the EL4-LILRB4 lymphoma model is summarized in Table 20 below and detailed as follows.
(a) The EL4-LILRB4 lymphoma tumor cells were maintained in vitro with DMEM medium supplemented with 10% fetal bovine serum at 37° C. in an atmosphere of 5% CO₂ in air. The cells in exponential growth phase were harvested and quantitated by cell counter before tumor inoculation.
(b) Each mouse was inoculated intravenously in the right rear flank region with EL4-LILRB4 lymphoma tumor cells (0.2×10\hat{ }6) in 0.2 ml of PBS for tumor development. The date of randomization was denoted as Day 0, dosing starts from Day 0.
(c) The randomization started when total flux reached approximately 10⁶ p/sec. 18 mice were enrolled in this study. All animals were randomly allocated to 3 study groups. Randomization were performed based on randomized block design.
(d) Tumor flux was measured twice per week. Dosing as well as tumor and body weight measurements were conducted in a Laminar Flow Cabinet.

TABLE 20
Study design for in vivo efficacy test in EL4-LILRB4 lymphoma
model

| | | Tumor | | | Dosing Frequency |
|---|---|---|---|---|---|
| Group | Treatment | model | Dose | ROA | & Duration |
| 1 | hIgG1 | EL4- | 3 mg/kg | i.p. | Q3D (Day 0, 3, 6), 3 |
| | | LILRB4 | | | doses |
| 2 | IO-202 | EL4- | 3 mg/kg | i.p. | Q3D (Day 0, 3, 6), 3 |
| | | LILRB4 | | | doses |

### 8.2. Results

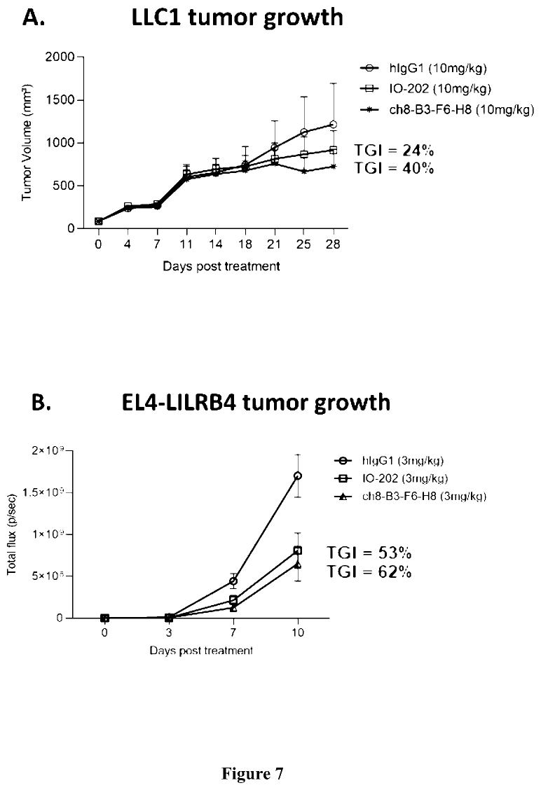

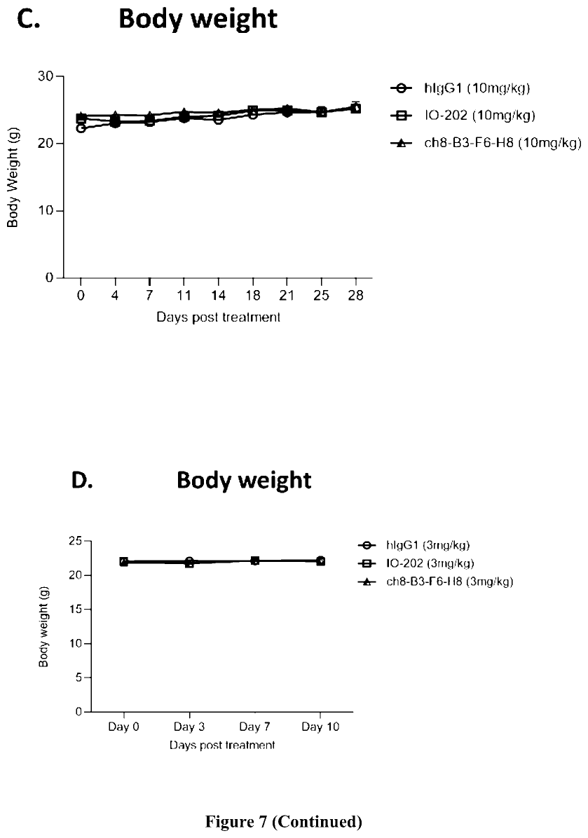

As shown in FIG. 7, ch8-B3-F6-H8 showed potent in vivo efficacy. For example, as shown in FIG. 7A, 10 mg/kg ch8-B3-F6-H8 in combination with 10 Gy radiotherapy significantly inhibited the LLC1 tumor growth in vivo with a tumor growth inhibition (TGI) of 40%, while the benchmark antibody (i.e., IO-202) only demonstrated 24% TGI in the LLC1 murine syngeneic model. As shown in FIG. 7B, 3 mg/kg ch8-B3-F6-H8 significantly inhibited the EL4-LILRB4 tumor growth in vivo with a TGI of 62%, while the benchmark antibody (i.e., IO-202) demonstrated a TGI of 53% in the EL4-LILRB4 syngeneic model. In addition, the body weights of the mice in each group in LLC1 model (FIG. 7C) and EL4-LILRB4 model (FIG. 7D) were not significantly changed.

### Example 9. Humanized Antibodies of 2-H1-D7-E5-D5 and 8-B3-F6-H8: Generation and Antibody Characterization

### 9.1 Humanized Antibody Generation

Chimeric antibodies ch2-H1-D7-E5-D5 and ch8-B3-F6-H8 were selected as the clones for humanization. Antibody sequences were aligned with human germline sequences to identify best fit model. Best matched human germline sequences were selected as the templates for humanization based on homology to the original mouse antibody sequences. Generally, humanization of an antibody was performed by comparing IMGT (https://www.imgt.org) human antibody heavy and light chain variable strain gene database, heavy chain and light chain variable strain genes with high homology with murine-derived antibody were selected as templates, and CDRs of murine-derived antibody were transplanted into corresponding human templates. A variable region sequence with the order FR1-CDR1-FR2-CDR2-FR3-CDR3-FR4 was formed. As required, the key amino acids in the skeleton sequence were reverted and mutated to those corresponding to murine antibodies to ensure the original affinity. The two humanized antibodies were designated as h2-H1-D7-E5-D5 and h8-B3-F6-H8, respectively, where the prefix “h” indicates “humanized”, the suffix “2-H1-D7-E5-D5”, for example, denotes the serial number of the humanized antibody clone of ch2-H1-D7-E5-D5. The amino acid sequence information of h2-H1-D7-E5-D5 and h8-B3-F6-H8 were shown in Table 21 above. As can be seen in Table 21, the CDRs of h2-H1-D7-E5-D5 are identical to its corresponding mouse monoclonal antibody 2-H1-D7-E5-D5; the CDRs of h8-B3-F6-H8 are identical to its corresponding mouse monoclonal antibody 8-B3-F6-H8 except for HCDR2. In particular, the amino acid sequence of HCDR2 of h8-B3-F6-H8 is as set forth in SEQ ID NO: 158 (INPESSAI), while the amino acid sequence of HCDR2 of the corresponding mouse monoclonal antibody 8-B3-F6-H8 is as set forth in SEQ ID NO: 42 (INPDSSAI). While not wishing to be bound by any particular theory, such a change in HCDR2 is believed to be favorable for binding affinity to human LILRB4.

### 9.2. Binding Affinity Tests of Humanized Antibodies

### 9.2.1. Protein-Based Affinity Test by Biacore (Biointron)

The parameters for the protein-based affinity test by Biacore (Biointron) were as follows:
Analyte: LILRB4;
Running buffer: HBS-EP⁺;
Flow Rate: 30 μL/min;
Capture: Abs, 10 μL/min for 60 s;
Injection of serial diluted LILRB4;
Contact time: 180 s, dissociation time: 400 s;
Regeneration: pH1.5 Gly, 30 μL/min for 30 s;
Method: Multiple cycle kinetics/affinity using capture;
Machine Model: Biacore 8K (GE)
Analysis Temperature: 25° C.

### 9.2.2. Protein-Based Affinity Test by ELISA

The protocol for the protein-based affinity test by ELISA is as follows:
(a) 100 pl/well diluted soluble protein (0.5 ug/mL) was added to Clear Flat-Bottom Immuno 96 well plate (Nunc, cat #442404), and incubated overnight at 4° C.
(b) The cells were washed for 3 times with PBS containing 0.05% Tween-20 (PBST/BSA).
(c) Block plates: 200 μl 2% BSA to each well and incubated for 1 hour at 37° C.
(d) Washed for 3 times with PBS containing 0.05% Tween-20 (PBST/BSA).
(e) 100 μl test antibodies (primary antibody final concentration: adjust the starting concentration of antibody to 1000 nM or 120 nM and serially dilute the antibody in a 1:3 ratio) were added to each well and incubated for 1 hour at 37° C.
(f) Washed for 3 times with PBS containing 0.05% Tween-20 (PBST/BSA).
(g) 100 μl HRP conjugated secondary antibody, dilution 1:10,000 was added, and incubated for 1 hour at 37° C.
(h) Washed for 3 times with PBS containing 0.05% Tween-20 (PBST/BSA).
(i) 100 μl Substrate Solution TMB (SURMODICS, cat #TMBS-1000-01) was added to each well and incubated for 30 mins at room temperature in dark.
(j) 50 μl Stop Solution was added to each well and read at 450 nm on MD SpectraMax iD3.

### 9.2.3. Cell-Based Binding Affinity on 293F-hLILRB4 Cells

The protocol for the cell-based binding affinity on 293F-hLILRB4 cells is similar to the protocol as described in Example 2.3.

### 9.2.4. Competitive Cell-Based Binding Affinity on 293F-hLILRB4 Cells

The protocol for the competitive cell-based binding affinity on 293F-hLILRB4 cells is as follows:
(a) Cells were harvested by centrifugation at 300 g for 3 mins and the supernatant was discarded.
(b) The cells were washed for 2 times with FACS buffer by centrifuging at 300 g for 3 mins and the supernatant was discarded.
(c) The cells were resuspended and seeded into assay plate (2*10⁵ cells/well) in 50 μl FACS buffer. 50 μl primary antibody was added to the plates at a serial of final concentrations (the working concentration of one competitor, for example, h2-H1-D7-E5-D5 was 10 μg/ml and other 3 antibodies including IO-202, NGM831 and h8-B3-F6-H8 were 50 μg/ml, 3 serial dilutions from 50 μg/ml, 10 gradients), incubated at 4° C. for 1 hour.
(d) Step (b) was repeated, and the cells were resuspended with 100 μl diluted 2nd antibody, incubated at 4° C. for 1 hour in the dark.
(e) Step (b) was repeated, and the cells were resuspended with 100 μl FACS buffer. The cells were kept in dark for FACS analysis.

### 9.2.5. Results

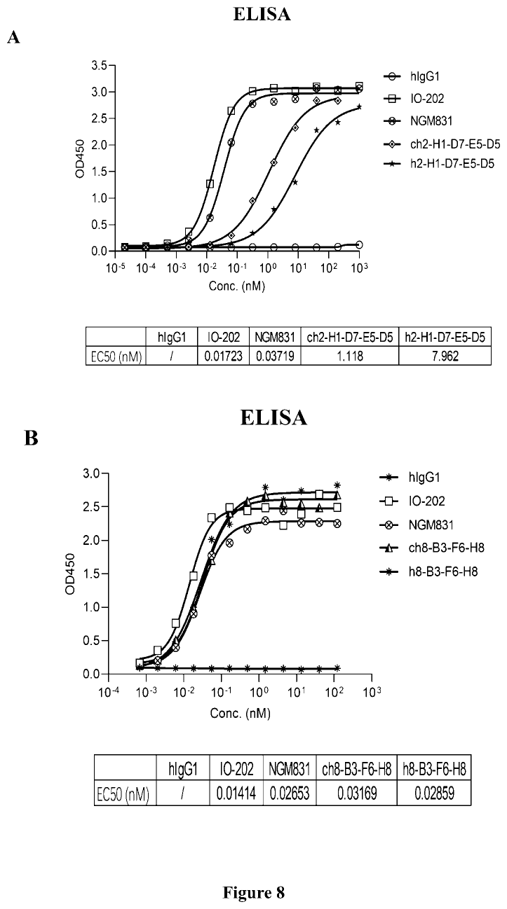

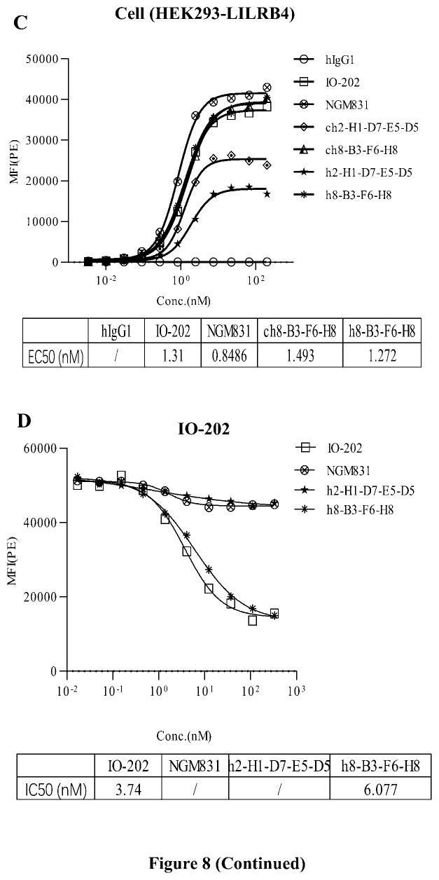

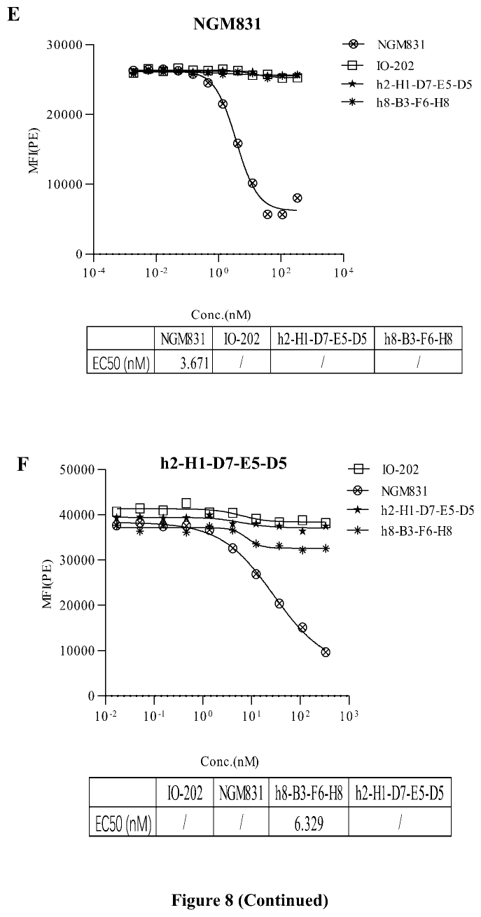

As shown in FIGS. 8A-C, the binding affinity of humanized antibody h8-B3-F6-H8 on 293F-hLILRB4 cell line was high and comparable with benchmark antibodies (IO-202 and NGM831) in ELISA and cell based binding assays, and humanized antibody h2-H1-D7-E5-D5 also showed binding on 293F-hLILRB4 cell line in ELISA and cell based binding assays. As shown in FIGS. 8D-F, h8-B3-F6-H8 had a similar binding epitope with IO-202 while h2-H1-D7-E5-D5 possessed a specific binding epitope.

### 9.3. Antibody Characterization: Mixed Lymphocyte Reaction (MLR) of Humanized Antibodies

The humanized antibodies h2-H1-D7-E5-D5 and h8-B3-F6-H8 prepared in Example 9.1 were used for the MLR. IO-202 and NGM831 were used as positive controls, and hIgG1 was used as a negative control. The protocol for the MLR of humanized antibodies is as follows:
(a) Day 0: Monocytes were isolated from PBMCs. 5×10⁶ cells were resuspended in 3 ml X-VIVO, supplemented with 2 U/ml of dendritic cell culture factor and then the cells were cultured in 6-well plate.
(b) Day 2:2 ml/well of fresh complete medium supplemented with 2 U/ml of dendritic cell culture factor were added.
(c) Day 5: Immature DCs were differentiated into tolDCs by the addition of 10 nmol/L dexamethasone and 100 nmol/L vitamin D3.
(d) Day 6:50-100 million PBMCs were ordered for T cell isolation on the next day.
(e) Day 7: After stimulated 48 hours, immature DCs were differentiated into mature dendritic cells and tolDCs, respectively.
(f) Day 7: T cells were isolated from PBMCs (about 15-30 million T cells isolated from 50-100 million PBMCs). Half of the isolated T cells were labeled with CFSE and the other half were not labeled.
(g) Day 7:0.2 million cells/well T cells and 0.025 million cells/well tolDCs were seeded in 96 well (tolDCs are nonadherent cells).
(h) Day 7: Cells, a mixture of DC and T cells were treated with PBS, hIgG1, IO-202, NGM831, h2-H1-D7-E5-D5 or h8-B3-F6-H8 (final concentration: 15 μg/ml), three replicates for each group. Also, two groups that T cell alone and tolDC alone were used as controls.
(i) Day 12: Supernatants were assayed with IFN-γ ELISA kit.

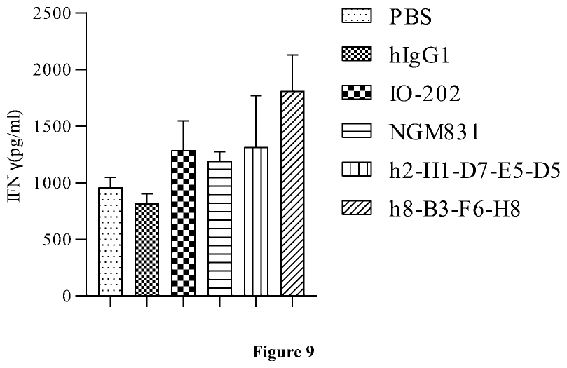

As shown in FIG. 9, humanized antibody h8-B3-F6-H8 reinvigorated tolDCs for priming T cell activation.

### 9.4. Antibody Characterization: Macrophage and T Cell Coculture Assay of Humanized Antibodies

The humanized antibodies h2-H1-D7-E5-D5 and h8-B3-F6-H8 prepared in Example 9.1 were used for the macrophage and T cell coculture assay. IO-202 and NGM831 were used as positive controls, and hIgG1 was used as a negative control. The protocol for the macrophage and T cell coculture assay is as follows:
(a) Monocytes were separated by EasySep™ Human CD14 Positive Selection Kit II (Stemcell, 17858).
(b) The monocytes were cultured for 5 days in X-VIVO (55 μm β-ME) and 100 ng/mL rhM-CSF. Half medium was replaced and cytokines were added every 2 to 3 days.
(c) 50 ng/ml TGF-β and rhIL-10 were added and cultured for additional 2 days.
(d) The cells were digested with Accutase enzyme cell detachment medium at 37° C. for 10 mins, centrifuged and resuspended in X-VIVO (55 μm β-ME).
(e) 3.3×10⁴, 1×10⁴/well macrophages were seeded and antibody (10 μg/mL) was added to a 96-well plate, incubated for 1 hour at 37° C., 5% CO₂.
(f) 30 ng/ml soluble CD3 antibody were added to the T cells. 1×10⁵/well T cells were seeded to 96-well plate, and incubated at 37° C., 5% CO₂ for 3 days.
(g) After that, the supernatant was collected by centrifugation for IFN-Y.

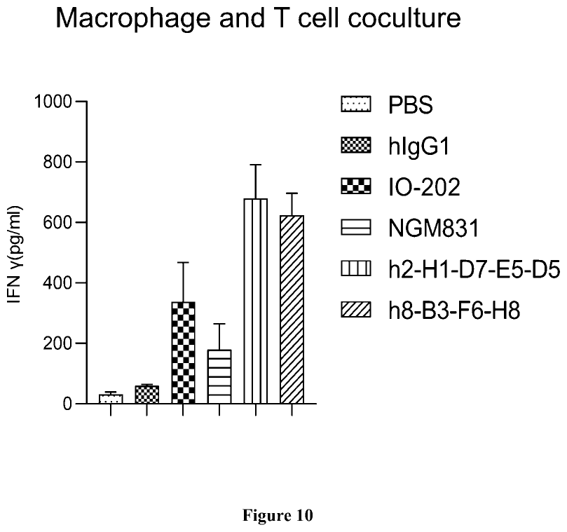

As shown in FIG. 10, humanized antibodies h2-H1-D7-E5-D5 and h8-B3-F6-H8 reversed macrophage-mediated T cell suppression.

### 9.5. Antibody Characterization: Cytotoxic CD8⁺ T Cell Killing Assay of Humanized Antibodies

The humanized antibodies h2-H1-D7-E5-D5 and h8-B3-F6-H8 prepared in Example 9.1 was used for the cytotoxic CD8⁺ T cell killing assay. The protocol for the Cytotoxic CD8⁺ T cell killing assay is as follows:
(a) CD3⁺ T cells were isolated from PBMC.
(b) 5×10³ target cells (THP-1 with labeled CFSE) were added to wells of a 96-well U-bottom plate.
(c) 1×10⁴ T cells were added to the wells (20:1 Effector: Target ratio).
(d) Target cells and T cells were mixed with final concentration 20 μg/ml.
(e) The cells were incubated for 20 hours at 37° C., 5% CO₂ for 18 hours.
(f) Finally, cells were washed and resuspended in 200 μL of staining buffer. Cells were acquired by FACS and percentages of CFSE labeled THP-1 cells were measured.

Percent of cell cytotoxicity was calculated according to the equation below:

$$Cell Cytotoxicity ( \% ) = 100 \% - \frac{AT}{NAT} \times 100 \%$$

(AT: percentages of CFSE labeled THP-1 cell in antibody treated group;
NAT: percentages of CFSE labeled THP-1 cell in negative control group)

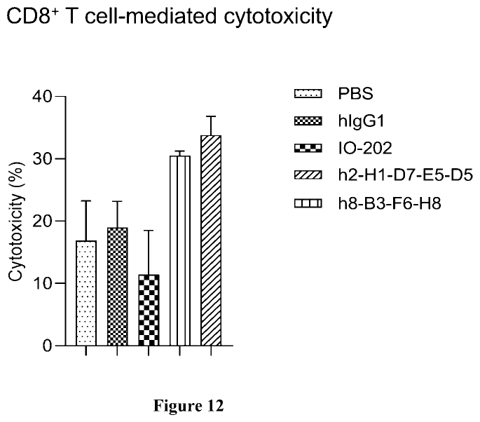

As shown in FIG. 12, humanized antibodies h2-H1-D7-E5-D5 and h8-B3-F6-H8 potentiated CD8⁺ T cell-mediated cytotoxicity on THP-1 cells.

### 9.6. Antibody Characterization: ADCC Assay and ADCP Assay of Humanized Antibodies

The humanized antibody h2-H1-D7-E5-D5 prepared in Example 9.1 was used for the ADCC assay and ADCP assay. The protocol for ADCC assay is similar to the protocol described in Example 3.1 and the protocol for ADCP assay is similar to the protocol described in Example 4.1, except that different test antibodies were used.

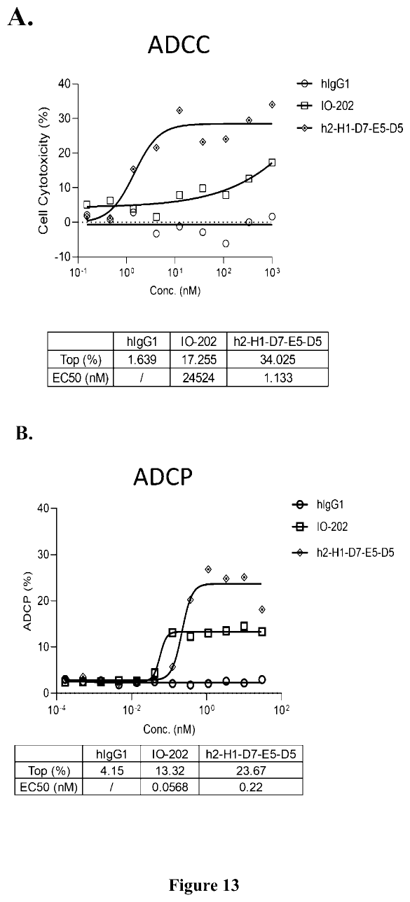

As shown in FIGS. 13A-B, the humanized antibody h2-H1-D7-E5-D5 showed significantly potent ADCC and ADCP effects on THP-1 cells and lower EC₅₀ compared with the benchmark antibody IO-202.

9.7. Antibody Characterization: In Vivo Efficacy Tests of Humanized Antibodies in the Treatment of EL4-LILRB4 Lymphoma in Female hLILRB1/hLILRB4 Transgenic Mice

The humanized antibodies h2-H1-D7-E5-D5 and h8-B3-F6-H8 prepared in Example 9.1 were used for the in vivo efficacy test in the treatment of EL4-LILRB4 lymphoma in female hLILRB1/hLILRB4 transgenic mice. IO-202 was used as a positive control, and hIgG1 was used as a negative control.

The protocol for in vivo efficacy assay in the EL4-LILRB4 lymphoma model is similar to the protocol described in Example 8.1.2, except for humanized antibodies h2-H1-D7-E5-D5 and h8-B3-F6-H8 were used as the test antibodies.

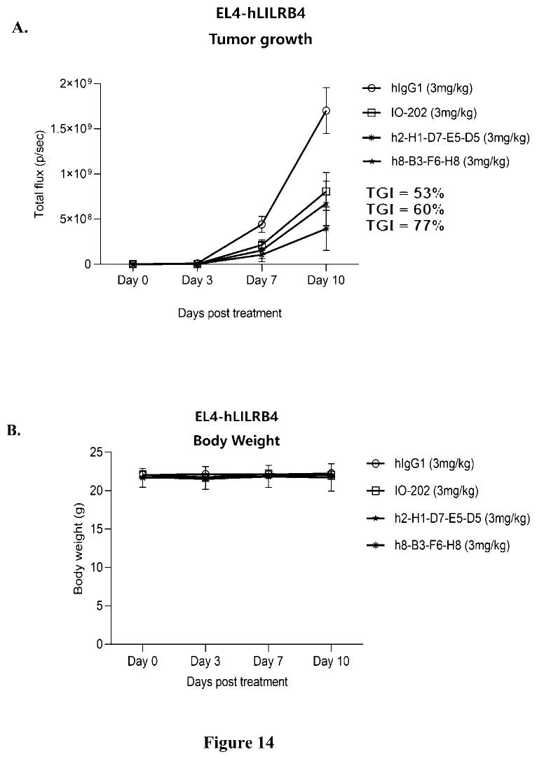

As shown in FIGS. 14A-B, the humanized antibodies h2-H1-D7-E5-D5 and h8-B3-F6-H8 showed potent in vivo efficacy compared to IO-202, and h2-H1-D7-E5-D5 had the strongest in vivo efficacy. In particular, as shown in FIG. 14A, 3 mg/kg h2-H1-D7-E5-D5 and 3 mg/kg h8-B3-F6-H8 significantly inhibited the EL4-LILRB4 tumor growth in vivo with a TGI of 77% and 60%, respectively, while the benchmark antibody IO-202 demonstrated a TGI of 53% in EL4-LILRB4 syngeneic model. In addition, as shown in FIG. 14B, the body weights of the mice in each group were not significantly changed.

What claimed is:

1. An antibody or antigen-binding fragment thereof which binds to LILRB4, comprising:
one, two or three heavy chain complementarity determining regions (HCDR1, HCDR2 and/or HCDR3) contained within any one of the heavy chain variable (VH) region sequences selected from the group consisting of SEQ ID NOs: 23, 31, 39, 47, 55, 63, 71, 79, 87, 95, 103, 111, 119, 127, 135, 143, 151, 154 and 156; and/or
one, two or three light chain complementarity determining regions (LCDR1, LCDR2 and LCDR3) contained within any one of the light chain variable (VL) region sequences selected from the group consisting of SEQ ID NOs: 24, 32, 40, 48, 56, 64, 72, 80, 88, 96, 104, 112, 120, 128, 136, 144, 152, 155 and 157.

2. The antibody or antigen-binding fragment thereof of claim 1, wherein the antibody or antigen-binding fragment comprises at least one heavy or light chain complementarity determining region (CDR) comprising an amino acid sequence selected from the group consisting of SEQ ID NOs: 17, 18, 19, 20, 21, 22, 25, 26, 27, 28, 29, 30, 33, 34, 35, 36, 37, 38, 41, 42, 43, 44, 45, 46, 49, 50, 51, 52, 53, 54, 57, 58, 59, 60, 61, 62, 65, 66, 67, 68, 69, 70, 73, 74, 75, 76, 77, 78, 81, 82, 83, 84, 85, 86, 89, 90, 91, 92, 93, 94, 97, 98, 99, 100, 101, 102, 105, 106, 107, 108, 109, 110, 113, 114, 115, 116, 117, 118, 121, 122, 123, 124, 125, 126, 129, 130, 131, 132, 133, 134, 137, 138, 139, 140, 141, 142, 145, 146, 147, 148, 149, 150 and 158.

3. The antibody or antigen-binding fragment thereof of claim 1 or 2, comprising one or two or three of HCDR1, HCDR2 and HCDR3 comprising an amino acid sequence selected from the group consisting of SEQ ID NOs: 17, 18, 19, 25, 26, 27, 33, 34, 35, 41, 42, 43, 49, 50, 51, 57, 58, 59, 65, 66, 67, 73, 74, 75, 81, 82, 83, 89, 90, 91, 97, 98, 99, 105, 106, 107, 113, 114, 115, 121, 122, 123, 129, 130, 131, 137, 138, 139, 145, 146, 147 and 158.

4. The antibody or antigen-binding fragment thereof of any one of the preceding claims, comprising one or two or three of LCDR1, LCDR2 and LCDR3 comprising an amino acid sequence selected from the group consisting of SEQ ID NOs: 20, 21, 22, 28, 29, 30, 36, 37, 38, 44, 45, 46, 52, 53, 54, 60, 61, 62, 68, 69, 70, 76, 77, 78, 84, 85, 86, 92, 93, 94, 100, 101, 102, 108, 109, 110, 116, 117, 118, 124, 125, 126, 132, 133, 134, 140, 141, 142, 148, 149 and 150.

5. The antibody or antigen-binding fragment thereof of any one of the preceding claims, comprising:
i. a HCDR1 comprising an amino acid sequence selected from the group consisting of SEQ ID NOs: 17, 25, 33, 41, 49, 57, 65, 73, 81, 89, 97, 105, 113, 121, 129, 137 and 145;
ii. a HCDR2 comprising an amino acid sequence selected from the group consisting of SEQ ID NOs: 18, 26, 34, 42, 50, 58, 66, 74, 82, 90, 98, 106, 114, 122, 130, 138, 146 and 158; and
iii. a HCDR3 comprising an amino acid sequence selected from the group consisting of SEQ ID NOs: 19, 27, 35, 43, 51, 59, 67, 75, 83, 91, 99, 107, 115, 123, 131, 139 and 147.

6. The antibody or antigen-binding fragment thereof of any one of the preceding claims, comprising:
i. a LCDR1 comprising an amino acid sequence selected from the group consisting of SEQ ID NOs: 20, 28, 36, 44, 52, 60, 68, 76, 84, 92, 100, 108, 116, 124, 132, 140 and 148;
ii. a LCDR2 comprising an amino acid sequence selected from the group consisting of SEQ ID NOs: 21, 29, 37, 45, 53, 61, 69, 77, 85, 93, 101, 109, 117, 125, 133, 141 and 149; and
iii. a LCDR3 comprising an amino acid sequence selected from the group consisting of SEQ ID NOs: 22, 30, 38, 46, 54, 62, 70, 78, 86, 94, 102, 110, 118, 126, 134, 142 and 150.

7. The antibody or antigen-binding fragment thereof of any one of the preceding claims, comprising:
i. a HCDR1 comprising an amino acid sequence as set forth in SEQ ID NO: 17, a HCDR2 comprising an amino acid sequence as set forth in SEQ ID NO: 18, and a HCDR3 comprising an amino acid sequence as set forth in SEQ ID NO: 19;
ii. a HCDR1 comprising an amino acid sequence as set forth in SEQ ID NO: 25, a HCDR2 comprising an amino acid sequence as set forth in SEQ ID NO: 26, and a HCDR3 comprising an amino acid sequence as set forth in SEQ ID NO: 27;
iii. a HCDR1 comprising an amino acid sequence as set forth in SEQ ID NO: 33, a HCDR2 comprising an amino acid sequence as set forth in SEQ ID NO: 34, and a HCDR3 comprising an amino acid sequence as set forth in SEQ ID NO: 35;
iv. a HCDR1 comprising an amino acid sequence as set forth in SEQ ID NO: 41, a HCDR2 comprising an amino acid sequence as set forth in SEQ ID NO: 42, and a HCDR3 comprising an amino acid sequence as set forth in SEQ ID NO: 43;
v. a HCDR1 comprising an amino acid sequence as set forth in SEQ ID NO: 41, a HCDR2 comprising an amino acid sequence as set forth in SEQ ID NO: 158, and a HCDR3 comprising an amino acid sequence as set forth in SEQ ID NO: 43;
vi. a HCDR1 comprising an amino acid sequence as set forth in SEQ ID NO: 49, a HCDR2 comprising an amino acid sequence as set forth in SEQ ID NO: 50, and a HCDR3 comprising an amino acid sequence as set forth in SEQ ID NO: 51;
vii. a HCDR1 comprising an amino acid sequence as set forth in SEQ ID NO: 57, a HCDR2 comprising an amino acid sequence as set forth in SEQ ID NO: 58, and a HCDR3 comprising an amino acid sequence as set forth in SEQ ID NO: 59;
viii. a HCDR1 comprising an amino acid sequence as set forth in SEQ ID NO: 65, a HCDR2 comprising an amino acid sequence as set forth in SEQ ID NO: 66, and a HCDR3 comprising an amino acid sequence as set forth in SEQ ID NO: 67;
ix. a HCDR1 comprising an amino acid sequence as set forth in SEQ ID NO: 73, a HCDR2 comprising an amino acid sequence as set forth in SEQ ID NO: 74, and a HCDR3 comprising an amino acid sequence as set forth in SEQ ID NO: 75;
x. a HCDR1 comprising an amino acid sequence as set forth in SEQ ID NO: 81, a HCDR2 comprising an amino acid sequence as set forth in SEQ ID NO: 82, and a HCDR3 comprising an amino acid sequence as set forth in SEQ ID NO: 83;
xi. a HCDR1 comprising an amino acid sequence as set forth in SEQ ID NO: 89, a HCDR2 comprising an amino acid sequence as set forth in SEQ ID NO: 90, and a HCDR3 comprising an amino acid sequence as set forth in SEQ ID NO: 91;
xii. a HCDR1 comprising an amino acid sequence as set forth in SEQ ID NO: 97, a HCDR2 comprising an amino acid sequence as set forth in SEQ ID NO: 98, and a HCDR3 comprising an amino acid sequence as set forth in SEQ ID NO: 99;
xiii. a HCDR1 comprising an amino acid sequence as set forth in SEQ ID NO: 105, a HCDR2 comprising an amino acid sequence as set forth in SEQ ID NO: 106, and a HCDR3 comprising an amino acid sequence as set forth in SEQ ID NO: 107;
xiv. a HCDR1 comprising an amino acid sequence as set forth in SEQ ID NO: 113, a HCDR2 comprising an amino acid sequence as set forth in SEQ ID NO: 114, and a HCDR3 comprising an amino acid sequence as set forth in SEQ ID NO: 115;
xv. a HCDR1 comprising an amino acid sequence as set forth in SEQ ID NO: 121, a HCDR2 comprising an amino acid sequence as set forth in SEQ ID NO: 122, and a HCDR3 comprising an amino acid sequence as set forth in SEQ ID NO: 123;
xvi. a HCDR1 comprising an amino acid sequence as set forth in SEQ ID NO: 129, a HCDR2 comprising an amino acid sequence as set forth in SEQ ID NO: 130, and a HCDR3 comprising an amino acid sequence as set forth in SEQ ID NO: 131;
xvii. a HCDR1 comprising an amino acid sequence as set forth in SEQ ID NO: 137, a HCDR2 comprising an amino acid sequence as set forth in SEQ ID NO: 138, and a HCDR3 comprising an amino acid sequence as set forth in SEQ ID NO: 139; or
xviii. a HCDR1 comprising an amino acid sequence as set forth in SEQ ID NO: 145, a HCDR2 comprising an amino acid sequence as set forth in SEQ ID NO: 146, and a HCDR3 comprising an amino acid sequence as set forth in SEQ ID NO: 147.

8. The antibody or antigen-binding fragment thereof of any one of the preceding claims, comprising:
i. a LCDR1 comprising an amino acid sequence as set forth in SEQ ID NO: 20, a LCDR2 comprising an amino acid sequence as set forth in SEQ ID NO: 21, and a LCDR3 comprising an amino acid sequence as set forth in SEQ ID NO: 22;
ii. a LCDR1 comprising an amino acid sequence as set forth in SEQ ID NO: 28, a LCDR2 comprising an amino acid sequence as set forth in SEQ ID NO: 29, and a LCDR3 comprising an amino acid sequence as set forth in SEQ ID NO: 30;
iii. a LCDR1 comprising an amino acid sequence as set forth in SEQ ID NO: 36, a LCDR2 comprising an amino acid sequence as set forth in SEQ ID NO: 37, and a LCDR3 comprising an amino acid sequence as set forth in SEQ ID NO: 38;
iv. a LCDR1 comprising an amino acid sequence as set forth in SEQ ID NO: 44, a LCDR2 comprising an amino acid sequence as set forth in SEQ ID NO: 45, and a LCDR3 comprising an amino acid sequence as set forth in SEQ ID NO: 46;
v. a LCDR1 comprising an amino acid sequence as set forth in SEQ ID NO: 52, a LCDR2 comprising an amino acid sequence as set forth in SEQ ID NO: 53, and a LCDR3 comprising an amino acid sequence as set forth in SEQ ID NO: 54;
vi. a LCDR1 comprising an amino acid sequence as set forth in SEQ ID NO: 60, a LCDR2 comprising an amino acid sequence as set forth in SEQ ID NO: 61, and a LCDR3 comprising an amino acid sequence as set forth in SEQ ID NO: 62;
vii. a LCDR1 comprising an amino acid sequence as set forth in SEQ ID NO: 68, a LCDR2 comprising an amino acid sequence as set forth in SEQ ID NO: 69, NO: 70;
viii. a LCDR1 comprising an amino acid sequence as set forth in SEQ ID NO: 76, a LCDR2 comprising an amino acid sequence as set forth in SEQ ID NO: 77, and a LCDR3 comprising an amino acid sequence as set forth in SEQ ID NO: 78;
ix. a LCDR1 comprising an amino acid sequence as set forth in SEQ ID NO: 84, a LCDR2 comprising an amino acid sequence as set forth in SEQ ID NO: 85, and a LCDR3 comprising an amino acid sequence as set forth in SEQ ID NO: 86;
x. a LCDR1 comprising an amino acid sequence as set forth in SEQ ID NO: 92, a LCDR2 comprising an amino acid sequence as set forth in SEQ ID NO: 93, and a LCDR3 comprising an amino acid sequence as set forth in SEQ ID NO: 94;
xi. a LCDR1 comprising an amino acid sequence as set forth in SEQ ID NO: 100, a LCDR2 comprising an amino acid sequence as set forth in SEQ ID NO: 101, and a LCDR3 comprising an amino acid sequence as set forth in SEQ ID NO: 102;
xii. a LCDR1 comprising an amino acid sequence as set forth in SEQ ID NO: 108, a LCDR2 comprising an amino acid sequence as set forth in SEQ ID NO: 109, and a LCDR3 comprising an amino acid sequence as set forth in SEQ ID NO: 110;
xiii. a LCDR1 comprising an amino acid sequence as set forth in SEQ ID NO: 116, a LCDR2 comprising an amino acid sequence as set forth in SEQ ID NO: 117, and a LCDR3 comprising an amino acid sequence as set forth in SEQ ID NO: 118;
xiv. a LCDR1 comprising an amino acid sequence as set forth in SEQ ID NO: 124, a LCDR2 comprising an amino acid sequence as set forth in SEQ ID NO: 125, and a LCDR3 comprising an amino acid sequence as set forth in SEQ ID NO: 126;
xv. a LCDR1 comprising an amino acid sequence as set forth in SEQ ID NO: 132, a LCDR2 comprising an amino acid sequence as set forth in SEQ ID NO: 133, and a LCDR3 comprising an amino acid sequence as set forth in SEQ ID NO: 134;
xvi. a LCDR1 comprising an amino acid sequence as set forth in SEQ ID NO: 140, a LCDR2 comprising an amino acid sequence as set forth in SEQ ID NO: 141, and a LCDR3 comprising an amino acid sequence as set forth in SEQ ID NO: 142; or
xvii. a LCDR1 comprising an amino acid sequence as set forth in SEQ ID NO: 148, a LCDR2 comprising an amino acid sequence as set forth in SEQ ID NO: 149, and a LCDR3 comprising an amino acid sequence as set forth in SEQ ID NO: 150.

9. The antibody or antigen-binding fragment thereof of any one of the preceding claims, comprising:
i. a HCDR1 comprising an amino acid sequence as set forth in SEQ ID NO: 17, a HCDR2 comprising an amino acid sequence as set forth in SEQ ID NO: 18, a HCDR3 comprising an amino acid sequence as set forth in SEQ ID NO: 19, a LCDR1 comprising an amino acid sequence as set forth in SEQ ID NO: 20, a LCDR2 comprising an amino acid sequence as set forth in SEQ ID NO: 21, and a LCDR3 comprising an amino acid sequence as set forth in SEQ ID NO: 22;
ii. a HCDR1 comprising an amino acid sequence as set forth in SEQ ID NO: 25, a HCDR2 comprising an amino acid sequence as set forth in SEQ ID NO: 26, a HCDR3 comprising an amino acid sequence as set forth in SEQ ID NO: 27, a LCDR1 comprising an amino acid sequence as set forth in SEQ ID NO: 28, a LCDR2 comprising an amino acid sequence as set forth in SEQ ID NO: 29, NO: 30;
iii. a HCDR1 comprising an amino acid sequence as set forth in SEQ ID NO: 33, a HCDR2 comprising an amino acid sequence as set forth in SEQ ID NO: 34, a HCDR3 comprising an amino acid sequence as set forth in SEQ ID NO: 35, a LCDR1 comprising an amino acid sequence as set forth in SEQ ID NO: 36, a LCDR2 comprising an amino acid sequence as set forth in SEQ ID NO: 37, and a LCDR3 comprising an amino acid sequence as set forth in SEQ ID NO: 38;
iv. a HCDR1 comprising an amino acid sequence as set forth in SEQ ID NO: 41, a HCDR2 comprising an amino acid sequence as set forth in SEQ ID NO: 42, a HCDR3 comprising an amino acid sequence as set forth in SEQ ID NO: 43, a LCDR1 comprising an amino acid sequence as set forth in SEQ ID NO: 44, a LCDR2 comprising an amino acid sequence as set forth in SEQ ID NO: 45, and a LCDR3 comprising an amino acid sequence as set forth in SEQ ID NO: 46;
v. a HCDR1 comprising an amino acid sequence as set forth in SEQ ID NO: 41, a HCDR2 comprising an amino acid sequence as set forth in SEQ ID NO: 158, a HCDR3 comprising an amino acid sequence as set forth in SEQ ID NO: 43, a LCDR1 comprising an amino acid sequence as set forth in SEQ ID NO: 44, a LCDR2 comprising an amino acid sequence as set forth in SEQ ID NO: 45, and a LCDR3 comprising an amino acid sequence as set forth in SEQ ID NO: 46;
vi. a HCDR1 comprising an amino acid sequence as set forth in SEQ ID NO: 49, a HCDR2 comprising an amino acid sequence as set forth in SEQ ID NO: 50, a HCDR3 comprising an amino acid sequence as set forth in SEQ ID NO: 51, a LCDR1 comprising an amino acid sequence as set forth in SEQ ID NO: 52, a LCDR2 comprising an amino acid sequence as set forth in SEQ ID NO: 53, NO: 54;
vii. a HCDR1 comprising an amino acid sequence as set forth in SEQ ID NO: 57, a HCDR2 comprising an amino acid sequence as set forth in SEQ ID NO: 58, a HCDR3 comprising an amino acid sequence as set forth in SEQ ID NO: 59, a LCDR1 comprising an amino acid sequence as set forth in SEQ ID NO: 60, a LCDR2 comprising an amino acid sequence as set forth in SEQ ID NO: 61, and a LCDR3 comprising an amino acid sequence as set forth in SEQ ID NO: 62;
viii. a HCDR1 comprising an amino acid sequence as set forth in SEQ ID NO: 65, a HCDR2 comprising an amino acid sequence as set forth in SEQ ID NO: 66, a HCDR3 comprising an amino acid sequence as set forth in SEQ ID NO: 67, a LCDR1 comprising an amino acid sequence as set forth in SEQ ID NO: 68, a LCDR2 comprising an amino acid sequence as set forth in SEQ ID NO: 69, and a LCDR3 comprising an amino acid sequence as set forth in SEQ ID NO: 70;
ix. a HCDR1 comprising an amino acid sequence as set forth in SEQ ID NO: 73, a HCDR2 comprising an amino acid sequence as set forth in SEQ ID NO: 74, a HCDR3 comprising an amino acid sequence as set forth in SEQ ID NO: 75, a LCDR1 comprising an amino acid sequence as set forth in SEQ ID NO: 76, a LCDR2 comprising an amino acid sequence as set forth in SEQ ID NO: 77, and a LCDR3 comprising an amino acid sequence as set forth in SEQ ID NO: 78;
x. a HCDR1 comprising an amino acid sequence as set forth in SEQ ID NO: 81, a HCDR2 comprising an amino acid sequence as set forth in SEQ ID NO: 82, a HCDR3 comprising an amino acid sequence as set forth in SEQ ID NO: 83, a LCDR1 comprising an amino acid sequence as set forth in SEQ ID NO: 84, a LCDR2 comprising an amino acid sequence as set forth in SEQ ID NO: 85, NO: 86;
xi. a HCDR1 comprising an amino acid sequence as set forth in SEQ ID NO: 89, a HCDR2 comprising an amino acid sequence as set forth in SEQ ID NO: 90, a HCDR3 comprising an amino acid sequence as set forth in SEQ ID NO: 91, a LCDR1 comprising an amino acid sequence as set forth in SEQ ID NO: 92, a LCDR2 comprising an amino acid sequence as set forth in SEQ ID NO: 93, and a LCDR3 comprising an amino acid sequence as set forth in SEQ ID NO: 94;
xii. a HCDR1 comprising an amino acid sequence as set forth in SEQ ID NO: 97, a HCDR2 comprising an amino acid sequence as set forth in SEQ ID NO: 98, a HCDR3 comprising an amino acid sequence as set forth in SEQ ID NO: 99, a LCDR1 comprising an amino acid sequence as set forth in SEQ ID NO: 100, a LCDR2 comprising an amino acid sequence as set forth in SEQ ID NO: 101, and a LCDR3 comprising an amino acid sequence as set forth in SEQ ID NO: 102;
xiii. a HCDR1 comprising an amino acid sequence as set forth in SEQ ID NO: 105, a HCDR2 comprising an amino acid sequence as set forth in SEQ ID NO: 106, a HCDR3 comprising an amino acid sequence as set forth in SEQ ID NO: 107, a LCDR1 comprising an amino acid sequence as set forth in SEQ ID NO: 108, a LCDR2 comprising an amino acid sequence as set forth in SEQ ID NO: 109, and a LCDR3 comprising an amino acid sequence as set forth in SEQ ID NO: 110;
xiv. a HCDR1 comprising an amino acid sequence as set forth in SEQ ID NO: 113, a HCDR2 comprising an amino acid sequence as set forth in SEQ ID NO: 114, a HCDR3 comprising an amino acid sequence as set forth in SEQ ID NO: 115, a LCDR1 comprising an amino acid sequence as set forth in SEQ ID NO: 116, a LCDR2 comprising an amino acid sequence as set forth in SEQ ID NO: 117, and a LCDR3 comprising an amino acid sequence as set forth in SEQ ID NO: 118;
xv. a HCDR1 comprising an amino acid sequence as set forth in SEQ ID NO: 121, a HCDR2 comprising an amino acid sequence as set forth in SEQ ID NO: 122, a HCDR3 comprising an amino acid sequence as set forth in SEQ ID NO: 123, a LCDR1 comprising an amino acid sequence as set forth in SEQ ID NO: 124, a LCDR2 comprising an amino acid sequence as set forth in SEQ ID NO: 125, and a LCDR3 comprising an amino acid sequence as set forth in SEQ ID NO: 126;
xvi. a HCDR1 comprising an amino acid sequence as set forth in SEQ ID NO: 129, a HCDR2 comprising an amino acid sequence as set forth in SEQ ID NO: 130, a HCDR3 comprising an amino acid sequence as set forth in SEQ ID NO: 131, a LCDR1 comprising an amino acid sequence as set forth in SEQ ID NO: 132, a LCDR2 comprising an amino acid sequence as set forth in SEQ ID NO: 133, and a LCDR3 comprising an amino acid sequence as set forth in SEQ ID NO: 134;
xvii. a HCDR1 comprising an amino acid sequence as set forth in SEQ ID NO: 137, a HCDR2 comprising an amino acid sequence as set forth in SEQ ID NO: 138, a HCDR3 comprising an amino acid sequence as set forth in SEQ ID NO: 139, a LCDR1 comprising an amino acid sequence as set forth in SEQ ID NO: 140, a LCDR2 comprising an amino acid sequence as set forth in SEQ ID NO: 141, and a LCDR3 comprising an amino acid sequence as set forth in SEQ ID NO: 142; or
xviii. a HCDR1 comprising an amino acid sequence as set forth in SEQ ID NO: 145, a HCDR2 comprising an amino acid sequence as set forth in SEQ ID NO: 146, a HCDR3 comprising an amino acid sequence as set forth in SEQ ID NO: 147, a LCDR1 comprising an amino acid sequence as set forth in SEQ ID NO: 148, a LCDR2 comprising an amino acid sequence as set forth in SEQ ID NO: 149, and a LCDR3 comprising an amino acid sequence as set forth in SEQ ID NO: 150.

10. The antibody or antigen-binding fragment thereof of any one of the preceding claims, comprising a VH region having an amino acid sequence as set forth in SEQ ID NOs: 23, 31, 39, 47, 55, 63, 71, 79, 87, 95, 103, 111, 119, 127, 135, 143, 151, 154 or 156, or a homologous sequence thereof having at least 80% sequence identity to SEQ ID NOs: 23, 31, 39, 47, 55, 63, 71, 79, 87, 95, 103, 111, 119, 127, 135, 143, 151, 154 or 156.

11. The antibody or antigen-binding fragment thereof of any one of the preceding claims, comprising a VL region having an amino acid sequence as set forth in SEQ ID NOs: 24, 32, 40, 48, 56, 64, 72, 80, 88, 96, 104, 112, 120, 128, 136, 144, 152, 155 or 157, or a homologous sequence thereof having at least 80% sequence identity to SEQ ID NOs: 24, 32, 40, 48, 56, 64, 72, 80, 88, 96, 104, 112, 120, 128, 136, 144, 152, 155 or 157.

12. The antibody or antigen-binding fragment thereof of any one of the preceding claims, comprising a VH/VL amino acid sequence pair selected from the group consisting of SEQ ID NOs: 23/24, 31/32, 39/40, 47/48, 55/56, 63/64, 71/72, 79/80, 87/88, 95/96, 103/104, 111/112, 119/120, 127/128, 135/136, 143/144, 151/152, 154/155, and 156/157.

13. The antibody or antigen-binding fragment thereof of any one of the preceding claims, further comprising one or more amino acid residue substitutions or modifications yet retains binding affinity to LILRB4.

14. The antibody or antigen-binding fragment thereof of claim 13, wherein at least one of the substitutions or modifications is in one or more of the CDR sequences of the VH region or VL region.

15. The antibody or antigen-binding fragment thereof of claim 13, wherein at least one of the substitutions or modifications is in one or more of the non-CDR sequences of the VH region or VL region.

16. The antibody or antigen-binding fragment of the proceeding claims, further comprising one or more non-natural amino acid (NNAA) substitution.

17. The antibody or antigen-binding fragment of claim 16, wherein the NNAA is capable of being conjugated.

18. The antibody or antigen-binding fragment thereof of any one of the preceding claims, having one or more binding properties to LILRB4 selected from the group consisting of:
i. being capable of specifically binding to human LILRB4 as measured by FACS assay;
ii. being capable of specifically binding to human LILRB4 and human LILRB3 as measured by FACS assay;
iii. being capable of specifically binding to human LILRB4 and cynomolgus LILRB4 as measured by FACS assay;
iv. being with potent ADCP effect on a human AML cell line;
v. being with potent ADCC effect on a human AML cell line;
vi. being capable of blocking the LILRB4-fibronectin interaction to reprogram tolerogenic dendritic cells to be mature dendritic cells which stimulate T cell activation as measured by FcγR stimulation assay;
vii. being capable of inducing TNF-α production;
viii. being capable of reprograming tolerogenic DC to activate T cells;
ix. being capable of reversing macrophage-mediated T cell suppression;
x. being capable of reversing THP-1-mediated T cell suppression; and
xi. being capable of potentiating CD8⁺ T cell-mediated cytotoxicity on THP-1 cells.

19. An antibody or antigen-binding fragment thereof, which competes for binding to LILRB4 with the antibody or antigen-binding fragment thereof any of the preceding claims.

20. The antibody or antigen-binding fragment thereof of any one of the preceding claims, which is a chimeric, a humanized or a human antibody or an antigen-binding fragment thereof.

21. The antibody or antigen-binding fragment thereof of any one of the preceding claims, which is a labeled antibody, a bivalent antibody, an anti-idiotypic antibody or a fusion protein.

22. The antibody or antigen-binding fragment thereof of any one of the preceding claims, which is a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a camelized single domain antibody, a nanobody, a domain antibody, or a bivalent domain antibody.

23. The antibody or antigen-binding fragment thereof of any one of the preceding claims, further comprising an Fc region, optionally an Fc region of human immunoglobulin (Ig), or optionally an Fc region of human IgG.

24. The antibody or antigen-binding fragment thereof of claim 23, wherein the Fc region is derived from human IgG1, IgG2, IgG3, or IgG4.

25. The antibody or antigen-binding fragment of claim 24, wherein the Fc region comprises an amino acid sequence as set forth in SEQ ID NO: 153.

26. The antibody or antigen-binding fragment thereof of any one of the preceding claims, wherein the light chain is a λ light chain or a κ light chain.

27. The antibody or antigen-binding fragment thereof of any one of the preceding claims, which is a bispecific or multi-specific antibody or an antigen-binding fragment thereof.

28. The antibody or antigen-binding fragment thereof of claim 27, which is capable of specifically binding to one or more additional antigens other than LILRB4, or a second epitope on LILRB4.

29. The antibody or antigen-binding fragment thereof of claim 28, wherein the one or more additional antigens other than LILRB4 are selected from the group consisting of CD3, CD16a, CD33, CD38, CD45, CD123, CD146, CD228, CLL-1, Flt3, TAF1, TgPRF, HVCN1, IL-6R, IL-11R, IL17A, IL-23R, IL-33, ILDR2, LAP, TSLP, TREM-1, ANGPT2, APOE, IFNAR, CypA, DOG-1, NKp30, CSF-1R, CCR2, LRRC15, mesothelin, Dickkopf2, DLL3, HER-2, C10orf54, TrkA, MEKK1, KRAS, ERK, XPO1, mTORC1/2, PAK4, NAMPT, ATR, EGFR, FGFR, VEGF, c-MET, Her2, Her3, CTLA4, GITA, CD112R, CD2, CD7, CD16, CD19, CD20, CD24, CD27, CD30, CD34, CD37, CD39, CD70, CD73, CD83, CD28, CD80 (B7-1), CD86 (B7-2), CD40, CD40L (CD154), CD47, SIRPα, CD122, CD137, CD137L, OX40 (CD134), OX40L (CD252), BCMA (e.g., BCMA02), PSMA, CLDN18 (e.g., CLDN18.2), NKG2C, 4-1BB, LIGHT, PVRIG, SLAMF7, HVEM, BAFFR, ICAM-1, 2B4, LFA-1, GITR, ICOS (CD278), ICOSLG (CD275), LAG3 (CD223), A2AR, B7-H3 (CD276), B7-H4 (VTCN1), B7-H5, BTLA (CD272), BTLA, CD160, CTLA-4 (CD152), GPRC5D, IDO1, IDO2, ILT3, TDO, KIR, LAIR-1, NOX2, PD-1, PD-L1, PD-L2, TIM-3, VISTA, SIGLEC-7 (CD328), SIGLEC-9 (CD329), SIGLEC-15, TIGIT, PVR (CD155), LILRB2, LILRB3, FLT3, FLT3L, TLR3, CLEC9A, DEC-205, STING, IL-12, IDO, and TGFβ.

30. The antibody or antigen-binding fragment thereof of any one of the preceding claims, which is linked to one or more conjugate moieties.

31. The antibody or antigen-binding fragment thereof of claim 30, wherein the conjugate moiety comprises a clearance-modifying agent, a chemotherapeutic agent, a toxin, a radioactive isotope, a lanthanide, a detectable label, a DNA-alkylator, a topoisomerase inhibitor, a tubulin-binder, a purification moiety or other anticancer drugs.

32. The antibody or antigen-binding fragment thereof of claim 30 or 31, wherein the conjugate moiety is covalently attached either directly or via a linker.

33. A pharmaceutical composition comprising the antibody or antigen-binding fragment thereof of any one of the preceding claims, and one or more pharmaceutically acceptable carriers.

34. A chimeric antigen receptor, comprising the antibody or antigen-binding fragment thereof of any one of claims 1-29, a transmembrane region and an intracellular signal region.

35. The chimeric antigen receptor of claim 34, wherein the transmembrane region comprises a transmembrane region of CD3, CD4, CD8 or CD28.

36. The chimeric antigen receptor of claim 34, wherein the intracellular signal region is selected from the group consisting of: an intracellular signal regions sequence of CD3, FcγRI, CD27, CD28, CD137, CD134, MyD88, CD40, CD278, TLRs, or a combination thereof.

37. The chimeric antigen receptor of any one of claims 34-36, wherein the antigen-binding fragment is a scFv.

38. The chimeric antigen receptor of any one of claims 34-37, wherein the chimeric antigen receptor is grafted onto an allogeneic cell, an autologous cell or a xenogeneic cell.

39. The chimeric antigen receptor of any one of claims 34-38, wherein the chimeric antigen receptor is grafted onto an immune effector cell.

40. The chimeric antigen receptor of any one of claims 34-39, wherein the chimeric antigen receptor is grafted onto a T cell, a natural killer cell, a macrophage cell, or a tumor-infiltrating lymphocyte.

41. An isolated polynucleotide encoding the antibody or antigen-binding fragment thereof of any one of claims 1-29, and/or the chimeric antigen receptor of any one of claims 34-40.

42. A vector comprising the isolated polynucleotide of claim 41.

43. A host expression system comprising the vector of claim 42 or having the polynucleotide of claim 41 integrated into genome thereof.

44. The host expression system of claim 43, which is a microorganism, a yeast, or a mammalian cell, optionally, wherein the microorganism is selected from the group consisting of E. coli and B. subtilis, wherein the yeast is Saccharomyces, and wherein the mammalian cell is selected from the group consisting of COS, CHO-S, CHO-K1, HEK-293, and 3T3 cells.

45. A virus comprising the vector of claim 42.

46. A kit comprising the antibody or antigen-binding fragment thereof of any one of claims 1-32 and/or the pharmaceutical composition of claim 33 and/or the chimeric antigen receptor of any one of claims 34-40, and a second therapeutic agent.

47. A method of expressing the antibody or antigen-binding fragment thereof of any one of claims 1-29 or the chimeric antigen receptor of any one of claims 34-40, comprising culturing the host expression system of claim 43 under the condition at which the antibody or antigen-binding fragment of any one of claims 1-29 or the chimeric antigen receptor of any one of claims 34-40 is expressed.

48. A method of treating, preventing or alleviating a disease, disorder or condition in a subject, comprising administering to the subject a therapeutically effective amount of the antibody or antigen-binding fragment thereof of any one of claims 1-32 and/or the pharmaceutical composition of claim 33 and/or the chimeric antigen receptor of any one of claims 34-40.

49. The method of claim 48, wherein the disease, disorder or condition is immune disease, inflammatory disease, cancer or neurological disease.

50. The method of claim 49, wherein the cancer is a solid tumor or hematologic tumor.

51. The method of any one of claims 48-50, wherein the disease, disorder or condition is selected from the group consisting of Kawasaki disease, T. gondii, multiple sclerosis, systematic Lupus erythematosus, lung cancer (e.g., non-small-cell lung cancer (NSCLC), small cell lung cancer (SCLC), adenocarcinoma of the lung, squamous cell carcinoma of the lung, Lewis lung carcinoma, or radiation therapy resistant Lewis lung carcinoma), peritoneal cancer, carcinoid cancer, bone cancer, pancreatic cancer, primitive neuroectodermal tumor, skin cancer, gallbladder cancer, cancer of the head or neck, squamous cell cancer, uterine cancer, ovarian cancer, rectal cancer, prostate cancer, bladder cancer (e.g., urothelial cancer), cancer of the anal region (e.g., anal squamous cell carcinoma), gastric or stomach cancer (e.g., gastrointestinal cancer), esophageal cancer, colon cancer, breast cancer, uterine cancer, liver cancer (e.g., hepatoblastoma, hepatocellular carcinoma/hepatoma, or hepatic carcinoma), cholangiocarcinoma, sarcoma, colorectal cancer, carcinoma of the fallopian tubes, salivary gland carcinoma, carcinoma of the cervix, endometrial or uterine carcinoma, osteosarcoma, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, cancer of the nasopharynx, sarcoma of soft tissue, polycythemia vera, cancer of the urethra, cancer of the penis, cancer of the kidney or ureter (e.g., rhabdoid tumor of the kidney), cutaneous T-cell lymphoma, medulloblastoma, nephroblastoma, myelodysplastic syndrome, chronic and non-chronic myeloproliferative disorder, choroid plexus papilloma, renal cell carcinoma, carcinoma of the renal pelvis, neoplasms of the central nervous system (CNS), soft tissue sarcoma (e.g., rhabdomyosarcoma, fibrosarcoma, Kaposi's sarcoma), spinal axis tumors, glioma (e.g., ependymoma, astrocytoma, anaplastic astrocytoma, oligodendroglioma, eye cancer (e.g., retinoblastoma), brain stem glioma, or mixed glioma such as oligoastrocytoma), brain tumor (e.g., glioblastoma/glioblastoma multiforme (GBM), non-glioblastoma brain tumor, or meningioma), melanoma (e.g., cutaneous or intraocular melanoma), thrombocythemia, mesothelioma, mycosis fungoides, Sezary syndrome, idiopathic myelofibrosis, solitary plasmacytoma, vestibular schwannoma, Ewing's sarcoma, chondrosarcoma, MYH associated polyposis, pituitary adenoma, pediatric cancers such as pediatric sarcomas (e.g., neuroblastoma, rhabdomyosarcoma, and osteosarcoma), hematological cancer, lymphoma, Hodgkin's lymphoma, non-Hodgkin's lymphoma, leukemia (e.g., lymphocytic/lymphoblastic leukemia), chronic or acute leukemia, mast cell leukemia, lymphocytic lymphomas, primary CNS lymphoma, chronic lymphocytic leukemia (CLL), acute lymphocytic leukemia (ALL), chronic myeloid leukemia (CML), acute myeloid leukemia (AML), chronic myelomonocytic leukemia (CMML), chronic lymphoblastic leukemia, acute lymphoblastic leukemia, hairy cell leukemia (HCL), Burkitt's lymphoma (BL), multiple myeloma (e.g., relapsed or refractory multiple myeloma), T or B cell lymphoma, mantle cell lymphoma (MCL) (e.g., relapsed or refractory mantle cell lymphoma), malignant melanoma, diffuse large B cell lymphoma (DLBCL), DLBCL that results from follicular lymphoma, high-grade B-cell lymphoma, primary mediastinal large B-cell lymphoma, follicular lymphoma (FL), and primary mediastinal B-cell lymphoma.

52. The method of any one of claims 48-51, wherein the subject is human.

53. The method of any one of claims 48-52, wherein the administration is through a parenteral route comprising subcutaneous, intraperitoneal, intravenous, intramuscular, or intradermal injection; or a non-parenteral route comprising transdermal, oral, intranasal, intraocular, sublingual, rectal, or topical.

54. The method of any one of claims 48-53, wherein the method further includes administering to the subject in need thereof an additional therapeutic agent.

55. The method of claim 54, wherein the additional therapeutic agent is selected from the group consisting of: an active agent, an imaging agent, a cytotoxic agent, and angiogenesis inhibitor, a kinase inhibitor, a co-stimulation molecule agonist, a co-inhibition molecule blocker, an adhesion molecule blocker, an anti-cytokine antibody or functional fragment thereof, a detectable label or reporter, an antimicrobial, a gene editing agent, a beta agonist, an viral RNA inhibitor, a polymerase inhibitor, an interferon, and a microRNA.

56. The method of claim 54, wherein the additional therapeutic agent is administered to the subject in need before, after or simultaneously with the antibody or antigen-binding fragment thereof of any one of claims 1-32 and/or the pharmaceutical composition of claim 33 and/or the chimeric antigen receptor of any one of claims 34-40.

57. A method of modulating LILRB4 activity in a LILRB4-expressing cell, comprising exposing the LILRB4-expressing cell to the antibody or antigen-binding fragment thereof of any of claims 1-32 and/or the pharmaceutical composition of claim 33 and/or the chimeric antigen receptor of any one of claims 34-40.

58. The method of claim 57, wherein the LILRB4-expressing cell is a dendritic cell, monocyte, macrophage, B cell, Treg, progenitor mast cell, endothelial cell, or osteoclast.

59. A method of inducing phagocytosis of a target cell in vivo or in vitro, comprising exposing the target cell to the antibody or antigen-binding fragment thereof of any of claims 1-32 and/or the pharmaceutical composition of claim 33 and/or the chimeric antigen receptor of any one of claims 34-40.

60. The method of claim 59, wherein the target cell is an antigen presenting cell, a cancer cell or a cell infected by a pathogen.

61. A method of inducing TNF-α production, comprising exposing the tolerogenic dendritic cell to the antibody or antigen-binding fragment thereof of any of claims 1-32 and/or the pharmaceutical composition of claim 33 and/or the chimeric antigen receptor of any one of claims 34-40.

62. A method of reprogramming a tolerogenic dendritic cell to a mature dendritic cell, comprising exposing the tolerogenic dendritic cell to the antibody or antigen-binding fragment thereof of any of claims 1-32 and/or the pharmaceutical composition of claim 33 and/or the chimeric antigen receptor of any one of claims 34-40.

63. A method of detecting presence or amount of LILRB4 in a sample, comprising contacting the sample with the antibody or antigen-binding fragment thereof of any of claims 1-32 and/or the pharmaceutical composition of claim 33 and/or the chimeric antigen receptor of any one of claims 34-40, and determining the presence or the amount of LILRB4 in the sample.

64. A method of diagnosing a LILRB4 related disease or condition in a subject, comprising: a) obtaining a sample from the subject; b) contacting the sample obtained from the subject with the antibody or antigen-binding fragment thereof of any of claims 1-32 and/or the pharmaceutical composition of claim 33; c) determining presence or amount of LILRB4 in the sample; and d) correlating the presence or the amount of LILRB4 to existence or status of the LILRB4 related disease or condition in the subject.

65. Use of the antibody or antigen-binding fragment thereof of any of claims 1-32 and/or the pharmaceutical composition of claim 33 and/or the chimeric antigen receptor of any one of claims 34-40 in the manufacture of a medicament for treating a LILRB4 related disease, disorder or condition in a subject.

66. Use of the antibody or antigen-binding fragment thereof of any of claims 1-32 and/or the pharmaceutical composition of claim 33 and/or the chimeric antigen receptor of any one of claims 34-40 in the manufacture of a diagnostic reagent for diagnosing a LILRB4 related disease, disorder or condition.

67. A kit comprising the antibody or antigen-binding fragment thereof of any of claims 1-32 and/or the pharmaceutical composition of claim 33 and/or the chimeric antigen receptor of any one of claims 34-40, useful in detecting LILRB4, optionally recombinant LILRB4, LILRB4 expressed on cell surface, or LILRB4-expressing cells.
